(19) United States
(12) Patent Application Publication
(10) Pub. No.: US 2025/0342903 A1
(43) Pub. Date: Nov. 6, 2025

(54) METHODS AND SYSTEMS FOR TRANSFORMER-BASED BIOLOGICAL SEQUENCE MODELS

(71) Applicant: Shape Therapeutics Inc., Seattle, WA (US)

(72) Inventors: Yingxin Cao, Seattle, WA (US); Yue Jiang, Los Angeles, CA (US); Ronald James Hause, JR., Seattle, WA (US); Bora Banjanin, Seattle, WA (US); Aydin Abiar, Seattle, WA (US)

(21) Appl. No.: 19/200,432

(22) Filed: May 6, 2025

Related U.S. Application Data
(60) Provisional application No. 63/643,354, filed on May 6, 2024.
(60) Provisional application No. 63/656,389, filed on Jun. 5, 2024.
(60) Provisional application No. 63/695,773, filed on Sep. 17, 2024.

(51) Int. Cl. G16B 15/10; A61K 48/00; C07H 21/02; G16B 20/30; G16B 30/00

(52) U.S. Cl. CPC G16B 15/10; A61K 48/0025; C07H 21/02; G16B 20/30; G16B 30/00

(57) ABSTRACT

A model comprising an encoder block and a decoder block are obtained. Nucleic acid sequence information for a scaffold formed between a gRNA and a target RNA including components corresponding to the gRNA and target RNA, and structural information comprising a base-pairing probability matrix for the scaffold, are inputted into the model. The encoder block comprises a first attention mechanism that receives the sequence information and the structural information. The decoder block includes a first sub-portion including a second and third attention mechanism and receives, as input, output generated from the encoder block. Output from the model is received, including predicted metrics for efficiency or specificity of deamination of target nucleotide positions in the target RNA by a deamination enzyme facilitated by hybridization of the gRNA to the target RNA.

### 1. CROSS REFERENCE TO RELATED APPLICATIONS

This patent application claims priority to U.S. Provisional Patent Application Ser. No. 63/643,354, filed May 6, 2024, U.S. Provisional Patent Application Ser. No. 63/656,389, filed Jun. 5, 2024, and U.S. Provisional Patent Application Ser. No. 63/695,773, filed Sep. 17, 2024, each of which is hereby incorporated by reference.

### 2. TECHNICAL FIELD

This specification describes technologies generally relating to predicting attributes and generating sequences for biological sequences, including guide RNAs, in particular using models comprising an encoder-decoder architecture.

### 3. BACKGROUND

RNA editing is a post-transcriptional process that recodes hereditary information by changing the nucleotide sequence of RNA molecules (Rosenthal, J Exp Biol. 2015 June; 218(12): 1812-1821). One form of post-transcriptional RNA modification is the conversion of adenosine-to-inosine (A-to-I), mediated by adenosine deaminase acting on RNA (ADAR) enzymes. Adenosine-to-inosine (A-to-I) RNA editing alters genetic information at the transcript level and is a biological process commonly conserved in metazoans. A-to-I editing is catalyzed by RNP complexes formed between guide RNAs (gRNAs) and adenosine deaminase acting on RNA (ADAR) enzymes. Such an intracellular RNA-editing mechanism potentially provides a versatile RNA-mutagenesis method for transcriptome manipulation. Another form of post-transcriptional RNA modification is the conversion of cytidine to uracil (C to U), mediated by RNP complexes formed between guide RNAs and apolipoprotein B editing complex (APOBEC) enzymes.

Current systems used to edit RNA have limitations which, in some embodiments, lead to aberrant effector activity, have a delivery barrier, unintended transcriptomic modifications, or immunogenicity. Further methods and systems for improved efficiency, specificity, and safety of targeted RNA editing are needed.

Recombinant adeno-associated viruses (rAAV) provide the leading platform for in vivo delivery of gene therapies. Current clinical trials employ a limited number of AAV capsids, primarily from naturally occurring human or primate serotypes such as AAV1, AAV2, AAV5, AAV6, AAV8, AAV9, AAVrh.10, AAV4rh.74, and AAVhu.67. These capsids often provide suboptimal targeting to tissues of interest, both due to poor infectivity of the tissue of interest and competing liver tropism. Increasing the dose to ensure infection of desired tissues can lead to dose-dependent liver toxicity. In addition, use of naturally-occurring capsids presents an immunological memory challenge—pre-immune patient populations are excluded from treatment and repeat dosing in a previously immune naïve patient is often not possible. Thus, there is a need for additional AAV capsids for use in gene therapy, in particular capsids that confer upon the rAAV high infectivity for specific tissues, such as muscle tissue and tissues in the central nervous system, and low liver tropism.

Regulatory elements, including promoters, enhancers, insulators, and the like operate in a sequence-specific fashion to direct transcription and/or translation. Discovery of sequence determinants of these regulatory elements, including tissue-specific activities, is made difficult by the fact that the genome is repetitive and has evolved to perform multiple functions. Furthermore, the human genome is too short to encode all combinations, orientations and spacings of approximately 1,639 human transcription factors in multiple independent sequence contexts. Thus, despite the information generated by genome-scale experiments, most sequence determinants that drive the activity of regulatory elements, including tissue specific activity, remain unknown. This is further complicated by the intricacy of binding site (e.g., transcription factor binding sites) grammar of individual regulatory elements. For instance, enhancers typically have clusters of such binding sites, the presence and arrangement of which is defined by a grammar that affects the overall ability of a given enhancer to promote gene expression and, in some instances, the tissue specificity of such gene expression.

### 4. SUMMARY

In general, there is a need for systems and methods for screening biological sequences, such as DNA, RNA, and protein sequences, for target properties for a given application. Additionally, there is a need for systems and methods for performing a priori design of biological sequences that are likely to have target properties for a given application and for using generative processes for sequence design and selection based on target properties, including input optimization processes. Given the above background, there is a need in the art for improved methods and systems for determining polymer sequences, such as sequences for guide RNAs, regulatory elements, and/or AAV capsid proteins. Provided herein, among other aspects, are machine learning approaches to evaluating, predicting, and/or designing polymer sequences using a model, e.g., a model including one or more encoder blocks, one or more decoder blocks, and/or where all or a portion of the model is pre-trained.

One aspect of the present disclosure provides a method for optimizing a model to predict a deamination efficiency or specificity. In some embodiments, the method is performed at a computer system comprising at least one processor and a memory storing at least one program for execution by the at least one processor. In some embodiments, the method includes obtaining a model comprising a plurality of parameters across a first block and a second block, where each of the first block and the second block comprises an attention mechanism, the plurality of parameters reflects, at least in part, pretraining information for a plurality of pretraining samples comprising, for each respective pretraining sample in the plurality of pretraining samples, a corresponding unlabeled nucleic acid sequence, and the model generates, responsive to inputting first test information comprising a respective nucleic acid sequence to the model, an indication of a structure or function associated with the nucleic acid sequence, or a representation thereof. In some embodiments, the method includes retraining the model using a plurality of training samples, where each respective training sample in the plurality of training samples comprises training information including (i) a corresponding training nucleic acid sequence for a guide RNA (gRNA)-target RNA scaffold formed between the gRNA and the target RNA when the gRNA hybridizes to the target RNA, and (ii) a corresponding training set of one or more metrics for an efficiency or specificity of deamination of a target nucleotide position in the target RNA by an Adenosine Deaminase Acting on RNA (ADAR) protein when facilitated by hybridization of the gRNA to the target RNA; thereby updating the plurality of parameters.

In some embodiments, the method further includes receiving, in electronic form, second test information comprising a nucleic acid sequence for a gRNA-target RNA scaffold formed between the gRNA and the target RNA when the gRNA hybridizes to the target RNA, and inputting the second test information into the retrained model, where the retrained model applies the updated plurality of parameters to the second test information to generate, as output from the retrained model, a test set of one or more metrics for an efficiency or specificity of deamination of the target nucleotide position in the target RNA by the ADAR protein when facilitated by hybridization of the gRNA to the target RNA.

Another aspect of the present disclosure provides a method for predicting a deamination efficiency or specificity comprising, at a computer system comprising at least one processor and a memory storing at least one program for execution by the at least one processor, obtaining a model comprising a first encoder block, a second encoder block, and a decoder block. In some embodiments, the first encoder block includes a first set of parameters, in a plurality of parameters of the model, that reflects, for each respective training sample in a plurality of training samples, information including (i) a first portion of a respective training nucleic acid sequence for a training scaffold, where the training scaffold is formed between a training guide RNA (gRNA) and a target RNA when the training gRNA hybridizes to the target RNA, and where the first portion corresponds to a nucleic acid sequence of the training gRNA, and (ii) a corresponding training set of one or more metrics for an efficiency or specificity of deamination of a target nucleotide position in the target RNA by an Adenosine Deaminase Acting on RNA (ADAR) protein when facilitated by hybridization of the training gRNA to the target RNA. In some embodiments, the second encoder block comprises a second set of parameters, in the plurality of parameters of the model, that reflects, for each respective training sample in the plurality of training samples, information comprising (i) a second portion of the respective training nucleic acid sequence for the training scaffold, wherein the second portion corresponds to the nucleic acid sequence of the target RNA, and (ii) the corresponding training set of one or more metrics. In some embodiments, the decoder block comprises a first portion and a second portion, where the first portion comprises a first attention mechanism that receives, as input, an output from the first encoder block and a second attention mechanism that receives, as input, an output from the second encoder block. In some embodiments, the method further includes inputting, into the model, information comprising a nucleic acid sequence for a test scaffold formed between a test gRNA and the target RNA when the test gRNA hybridizes to the target RNA, and receiving, as output from the model, a predicted set of one or more metrics for the efficiency or specificity of deamination of the target nucleotide position in the target RNA by the ADAR protein when facilitated by hybridization of the test gRNA to the target RNA.

Another aspect of the present disclosure provides a method for predicting a deamination efficiency or specificity comprising, at a computer system comprising at least one processor and a memory storing at least one program for execution by the at least one processor, obtaining a model comprising a first encoder block, a second encoder block, and a decoder block. In some embodiments, the first encoder block comprises a first set of parameters, in a plurality of parameters of the model, the second encoder block comprises a second set of parameters, in the plurality of parameters of the model, and the decoder block comprises a third set of parameters, in the plurality of parameters of the model. In some embodiments, the method further includes inputting, into the model, information comprising a nucleic acid sequence for a guide RNA (gRNA)-target RNA scaffold formed between the gRNA and the target RNA when the gRNA hybridizes to the target RNA. In some embodiments, the first encoder block (i) receives, as input, a first portion of the nucleic acid sequence for the gRNA-target RNA scaffold that corresponds to a sequence of the gRNA, or a representation thereof, and (ii) generates, as output, a representation of the first portion of the nucleic acid sequence. In some embodiments, the second encoder block (i) receives, as input, a second portion of the nucleic acid sequence for the gRNA-target RNA scaffold that corresponds to a sequence of the target RNA, or a representation thereof, and (ii) generates, as output, a representation of the second portion of the nucleic acid sequence. In some embodiments, the decoder block comprises a first portion and a second portion, where the first portion comprises a first attention mechanism that receives, as input, the output from the first encoder block and a second attention mechanism that receives, as input, the output from the second encoder block. In some embodiments, the method further includes receiving, as output from the model, a predicted set of one or more metrics for the efficiency or specificity of deamination of the target nucleotide position in the target RNA by the ADAR protein when facilitated by hybridization of the test gRNA to the target RNA.

Yet another aspect of the present disclosure provides a method for predicting a deamination efficiency or specificity at one or more target nucleotide positions of a target RNA. In some embodiments, the method is performed at a computer system comprising at least one processor and a memory storing at least one program for execution by the at least one processor. In some embodiments, the method includes inputting information about a target-guide scaffold formed between a guide RNA (gRNA) and the target RNA when the gRNA hybridizes to the target RNA into a model to receive as output from the model a predicted set of one or more metrics for an efficiency or specificity of deamination of the one or more target nucleotide positions in the target RNA by a deamination enzyme when facilitated by hybridization of the gRNA to the target RNA. In some embodiments, the information comprises a nucleotide sequence of the gRNA, a nucleotide sequence of the target RNA comprising the one or more target nucleotide positions, and structural information about the target-guide scaffold. In some embodiments, the model includes: a first portion comprising one or more encoder blocks that attend to a representation of the nucleotide sequence of the gRNA, a representation of the nucleotide sequence of the target RNA, and a representation of the structural information about the target-guide scaffold to generate one or more embeddings; and a second portion comprising one or more decoder blocks that attend to the one or more embeddings to generate the predicted set of one or more metrics.

Still another aspect of the present disclosure provides a computer system including one or more processors and a non-transitory computer-readable medium including computer-executable instructions that, when executed by the one or more processors, cause the processors to perform any of the methods and/or embodiments disclosed above.

Yet another aspect of the present disclosure provides a non-transitory computer-readable storage medium having stored thereon program code instructions that, when executed by a processor, cause the processor to perform any of the methods and/or embodiments disclosed above.

The systems, methods, and non-transitory computer readable storage medium of the present invention have other features and advantages that will be apparent from, or are set forth in more detail in, the accompanying drawings, which are incorporated herein, and the following Detailed Description, which together serve to explain certain principles of exemplary embodiments of the present invention.

### 5. BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A, 1B, 1C, and 1D collectively illustrate an example computer system for predicting a deamination efficiency or specificity, and/or optimizing a model to predict the same, in accordance with various embodiments of the present disclosure.

FIG. 2 provides a flowchart illustrating example methods for optimizing a model to predict a deamination efficiency or specificity, in which dashed boxes indicate optional features, in accordance with some embodiments of the present disclosure.

FIGS. 3A and 3B collectively illustrate example schematics for a transformer-based model comprising an encoder-decoder architecture, in accordance with some embodiments of the present disclosure.

FIGS. 4A and 4B collectively illustrate example schematics of applications for a transformer-based model comprising an encoder-decoder architecture, in accordance with some embodiments of the present disclosure. FIG. 4A illustrates an example schematic for predictive sequence modeling with a transformer-based model, in accordance with some embodiments of the present disclosure. FIG. 4B illustrates an example schematic for generative modeling with a transformer-based model, in accordance with some embodiments of the present disclosure.

FIGS. 5A and 5B collectively provide a flowchart illustrating example methods for predicting a deamination efficiency or specificity, in which dashed boxes indicate optional features, in accordance with some embodiments of the present disclosure.

FIG. 6 provides a flowchart illustrating example methods for predicting a deamination efficiency or specificity, in which dashed boxes indicate optional features, in accordance with some embodiments of the present disclosure.

FIG. 7 illustrates example schematics for a transformer-based model comprising an encoder-decoder architecture, in accordance with some embodiments of the present disclosure.

FIG. 8 illustrates improved performance of a transformer model trained using a training dataset that combines sparse data for a large number of target RNAs and deep data for a small number of single target RNAs, in accordance with an embodiment of the present disclosure.

FIG. 9 illustrates improved performance of a transformer model when incorporating, as input, as base-paring probability matrix, in accordance with an embodiment of the present disclosure.

FIG. 10 illustrates example schematics for a transformer-based model comprising an encoder-decoder architecture, in accordance with some embodiments of the present disclosure.

FIG. 11 illustrates example schematics for a transformer-based model comprising an encoder-decoder architecture, in accordance with some embodiments of the present disclosure.

FIGS. 12A and 12B illustrate example schematics for obtaining and using embeddings from an embedding model, such as an encoder, in accordance with some embodiments of the present disclosure.

FIGS. 13A and 13B collectively illustrate a flowchart illustrating example methods for predicting a deamination efficiency or specificity at one or more target nucleotide positions of a target RNA, in which dashed boxes indicate optional features, in accordance with some embodiments of the present disclosure.

It should be understood that the appended drawings are not necessarily to scale, presenting a somewhat simplified representation of various features illustrative of the basic principles of the invention.

### 6. DETAILED DESCRIPTION

### 6.1. Introduction

Guide RNAs. Personalized medicine for the treatment of monogenic diseases requires a rapid, cost-effective drug discovery process that is safe, programmable, and precise. The recruitment of endogenous adenosine deaminase acting on RNA (ADAR) enzymes by guide RNAs (gRNAs) antisense to a target transcript can allow for precise adenosine-to-inosine (A-to-I) editing at the RNA level, which is interpreted by the cellular machineries as an adenosine-to-guanosine substitution. This process, known as ADAR editing, plays a role in regulating the innate immune system by marking endogenous dsRNA structures as “self.” However, its therapeutic potential has been limited due to two factors: ADAR's natural preference for certain primary and secondary structural dsRNA substrates; and its proclivity to edit multiple adenosines within a given dsRNA substrate. Here, we demonstrate the power of machine learning (ML) to engineer novel gRNAs for challenging targets and rapidly identify gRNAs de novo to any target of interest.

Natural RNA substrates of ADAR and apolipoprotein B editing complex (APOBEC) are edited with high selectivity and efficiency due to precise higher order structures, e.g., secondary, tertiary, and quaternary structures formed between the RNA substrates, the gRNA, and the enzyme. In certain instances, guide RNA (gRNA) sequences can be designed such that they form gRNA-target scaffolds with the target RNAs to be edited, which are double-stranded RNA (dsRNA) substrates that bear unique structural features that help guide ADAR or APOBEC-mediated editing of the target sequence. Such an intracellular RNA-editing mechanism can be exploited, e.g., to edit mutations found in various genetic diseases at the mRNA level, and without modifying the genome of a patient. However, conventional systems used to edit RNA have limitations that can lead to aberrant effector activity, present delivery barriers, unintended transcriptomic modifications, and/or immunogenicity. In addition, the space from which such gRNA sequences can be selected is prohibitively large for conventional design and screening methodologies.

Therapeutic RNA editing using ADAR or APOBEC enzymes, e.g., by redirecting natural ADAR or APOBEC enzymes or by delivering exogenous ADAR or APOBEC enzymes, offers promise as a safe alternative to gene therapies that operate by altering the subject's genome. For example, some gene therapies introduce DNA breaks in the host's genome, which are repaired to introduce a permanent change in the host's genome. Imprecise editing by these gene therapies, for example by introducing an unintended mutation at a target site or any alteration at an off-target site, can thereby permanently harm the host's genome. RNA editing, by contrast, transiently alters the flow of genetic information in the host by editing RNA, e.g., messenger RNA (mRNA), without permanently altering the host's genome. Further, RNA editing strategies that redirect endogenous ADAR or APOBEC enzymes do not require introduction of exogenous proteins, which further complicates therapeutic delivery and risks further immunogenetic responses in the host.

However, ADAR and APOBEC enzymes possess inherent editing promiscuity. To date, sequence preferences and deterministic rules for how gRNA mediate result in various editing performances remain poorly understood. This is complicated by the fact the ADAR and APOBEC interactions with nucleic acids are influenced by tertiary nucleic acid structure and quaternary protein-nucleic acid structures, rather than just primary nucleic acid sequence.

For example, efforts to predict the editing preference of ADAR proteins for different dsRNA substrates have shown that ADAR editing activity, in some instances, not only tolerates various mismatches, bulges, loops, and other secondary and tertiary structural features, but also exhibits improved performance as a result of such deviations from perfect base-pairing. See, for instance, Liu et al., “Learning cis-regulatory principles of ADAR-based RNA editing from CRISPR-mediated mutagenesis.” Nat Commun. 2021; 12(1):2165, which is hereby incorporated herein by reference in its entirety. Moreover, gRNAs for ADAR editing can range from as small as about 20 nucleotides to about 151 nucleotides or more, and have further been shown, in certain instances, to tolerate mismatches at up to 50-60% of possible editing sites while still allowing recognition by the ADAR protein. See, for instance, Aquino-Jarquin, “Novel engineered programmable systems for ADAR-mediated RNA editing,” Mol. Ther. Nucleic Acids, 19:1065-72 (2020); Eggington et al., “Predicting sites of ADAR editing in double-stranded RNA,” Nat. Commun., 2(1):319 (2011), each of which is hereby incorporated herein by reference in its entirety.

Thus, for an example target RNA having 150 nucleotides, a conservative estimate of the space from which a corresponding gRNA sequence can be selected would be on the order of 10\hat{ }27, where any 10% of the positions in the gRNA sequence of 150 nucleotides are substituted, and assuming only single-base mismatches (e.g., A, C, G, or T) at each mutated position in the gRNA sequence. As another example, assuming only single-base mismatches over 10% of the gRNA sequence, the corresponding space for a target RNA having only 50 nucleotides still includes more than half of a billion potential gRNAs. However, in practice, the space from which the corresponding gRNA sequence for a given target RNA is selected is much larger than these estimates, given that the structural features that regulate ADAR editing specificity and efficiency are far more complex than simple base substitutions, including insertions and/or deletions, and considering that potential gRNA candidates include varying lengths that can be shorter or longer than the target RNA or target RNA region of interest. In some such cases, the space to be interrogated for a single gRNA corresponding to a single target RNA is at least 10\hat{ }30, 10\hat{ }40, 10\hat{ }50, or greater. Conventional methods for in vitro, in vivo, and in silico gRNA screening cannot properly evaluate such large space to identify optimal gRNA sequences. As such, improved methods and systems for identifying and/or designing gRNA sequences are needed.

These problems are attractive computational challenges for machine learning (ML). The problem compounds when considering the similarly enormous number of possible RNA editing sites in animals, such as mammals. In particular, more than 100 million adenosine to inosine (A-to-I) editing sites are estimated to occur in humans, and a further 50,000 sites are estimated to occur in mice. See, for instance, Kim et al., “RNA editing at a limited number of sites is sufficient to prevent MDA5 activation in the mouse brain.” PLOS Genetics. 2021; 17(5):e1009516, which is hereby incorporated herein by reference in its entirety. Given the sheer number of potential candidate gRNAs for any given RNA (e.g., mRNA) target, and the sheer number of potential RNA (e.g., mRNA) targets that contain A-to-I editing sites, a large-scale design or optimization of potential gRNAs for ADAR-mediated editing would be impossible to perform with any breadth. Moreover, with such a large candidate space, it would be impossible to perform a sufficient number of in vitro screening assays to sample the space to even identify an optimal starting point for tuning gRNA performance.

Thus, there is a need in the art for machine learning models that provide the ability to screen many more guides in silico, compared to in vitro approaches, to perform a priori design of sequences that enable specific and efficient editing of targets, and to use generative processes for guide design and selection based on target properties, including input optimization processes.

Variant capsid proteins. In some implementations, engineered capsids, engineered capsid polypeptides, and 581-589 regions of capsid polypeptides confer tissue tropism for specific tissues or a combination thereof (e.g., liver, CNS (cortex forebrain, cortex occipital, cortex temporal, thalamus, hypothalamus, substantia nigra, hippocampus DG, hippocampus CA1, hippocampus CA3, cerebellum), skeletal muscle, heart, lung, spleen, lymph node, bone marrow, mammary gland, skin, adrenal gland, thyroid, colon, sciatic nerve, and/or and spinal cord tissues) to a viral capsid. Current gene therapies utilize AAV viruses with wild type AAV capsid polypeptides. These therapies suffer from a lack of tissue specific tropism and, as such, can exhibit poor biodistribution, non-specific tissue tropism, or both. Even upon accumulation in target tissues, wild type AAV, such as wild type AAV9, can exhibit poor tissue-specific transduction. The rAAVs disclosed herein, and the systems and methods for generating the same, having variant AAV5 viral protein capsid polypeptide sequences, can display tissue and cell-type specific tropism (e.g., high transduction of specific tissue cells), decreased off-target tissue accumulation and infection (e.g., de-targeting), reduced capacity to pre-existing immunity, or any combination thereof. These attributes allow for reduction in clinical dose and a concomitant decrease in dose-dependent toxic side effects as well as increased manufacturability.

For example, engineered capsids comprising engineered capsid polypeptides with 581-589 regions for tissue-specific delivery of a payload (e.g., a polynucleotide, such as a transgene) encapsidated by the engineered capsid. Recombinant AAVs comprising VP capsid polypeptides with 581-589 regions engineered for tissue specificity can be used to specifically infect a target tissue. Using tissue-tropic rAAV viral capsids for payload delivery provides numerous advantages over using adeno-associated virus (AAV) viral capsids that lack tissue tropism including reduced toxicity, lower dose needed to produce a therapeutic effect, wider therapeutic window, and reduced immune response. Furthermore, tissue-specific payload delivery can enable targeted therapies even when administering systemically. For example, a target tissue-tropic AAV capsid can be systemically administered to specifically deliver a payload to the target tissue for treatment of a disease specific to the target tissue. In another example, a target tissue-tropic AAV capsid can be systemically administered to specifically deliver a payload to a specific organ for treatment of a target tissue disease. In some embodiments, a target tissue-tropic AAV capsid of the present disclosure can be systemically administered to specifically deliver a payload to target cell subtypes for treatment of a target tissue disease.

In some embodiments, a tissue-tropic capsid of the present disclosure is tissue-tropic for one or more tissues in a plurality of tissues including, but not limited to, liver, CNS (cortex forebrain, cortex occipital, cortex temporal, thalamus, hypothalamus, substantia nigra, hippocampus DG, hippocampus CA1, hippocampus CA3, cerebellum), skeletal muscle, heart, lung, spleen, lymph node, bone marrow, mammary gland, skin, adrenal gland, thyroid, colon, sciatic nerve, and/or and spinal cord tissues. Additionally or optionally, a tissue-tropic capsid further displays enhanced transduction of one or more cell subtypes for any one or more tissues in the plurality of tissues.

In an illustrative embodiment, variation is introduced into each of residues 581 to 589 of a variant capsid protein. Each of the 20 natural amino acids is introduced at each of the 9 positions of the 581-589 region, providing a theoretical library diversity of 20⁹ (20\hat{ }9; approximately 5×10¹¹) unique sequence variants.

In some implementations, the 581-589 region targeted for engineering is the most likely to interact with target cell receptors, and relatively tolerant to changes without disrupting capsid assembly. Unlike earlier approaches that add unstructured peptides that protrude above the capsid 3-fold axis of symmetry, the approach introduces sequence diversity that alters the characteristics of the binding pocket. In addition, this approach may change the overall structure of the receptor-binding trimer, allowing for altered allosteric interactions outside the binding pocket (e.g., AAVR PKD1). Introduced diversity is non-random, thereby reducing missense and frameshifts of randomized libraries.

Thus, there is a need in the art for machine learning models that provide the ability to screen capsid polypeptide sequences for tissue tropism in silico, to perform a priori design of sequences that enable specific and efficient delivery, and to use generative processes for sequence design and selection based on target properties, including input optimization processes.

Regulatory elements. In some embodiments, regulatory elements regulate (e.g., modulate, coordinate, or otherwise impact) the expression of one or more sequences in a cell. In some embodiments, regulatory elements include nucleotide sequences, such as promoters, enhancers, terminators, polyadenylation sequences, and/or introns. In some embodiments, regulatory elements affect coding sequences in the cell. In some implementations, engineered regulatory elements are used to produce a therapeutic effect, such as to inhibit overexpression or enhance under-expression and/or to activate or silence gene expression for gene therapy applications.

### 6.2. Definitions

Unless defined otherwise, all technical and scientific terms used herein have the meaning commonly understood by one of ordinary skill in the art to which the invention pertains.

As used herein, the term “engineered guide RNA” can be used interchangeably with “guide RNA” and refers to a designed polynucleotide that is at least partially complementary to a target RNA. An engineered guide RNA of the present disclosure can be used to facilitate modification of the target RNA. Modification of the target RNA includes alteration of RNA splicing, reduction or enhancement of protein translation, target RNA knockdown, target RNA degradation, and/or ADAR mediated RNA editing of the target RNA. In some cases, guide RNAs facilitate ADAR mediated RNA editing for the purpose of target RNA knockdown, downstream protein translation reduction or inhibition, downstream protein translation enhancement, correction of mutations (including correction of any G to A mutation, such as missense or nonsense mutations), introduction of mutations (e.g., introduction of an A to I (read as a G by cellular machinery) substitution), or alter the function of any adenosine containing a regulatory motif (e.g., polyadenylation signal, miRNA binding site, etc.). In some cases, a guide RNA can effect a functional outcome (e.g., target RNA modulation, downstream protein translation) via a combination of mechanisms, for example, ADAR-mediated RNA editing and binding and/or degrading target RNA. In some cases, a guide RNA can facilitate introduction of mutations at sites targeted by enzymes in order to modify the affinity of such enzymes for targeting and cleaving such sites. The guide RNAs of this disclosure can contain one or more structural features. A structural feature can be formed from latent structure in latent (unbound) guide RNA upon hybridization of the engineered latent guide RNA to a target RNA. Latent structure refers to a structural feature that forms or substantially forms only upon hybridization of a guide RNA to a target RNA. For example, upon hybridization of the guide RNA to the target RNA, the latent structural feature is formed in the resulting double stranded RNA (also referred herein as guide-target RNA scaffold). In such cases, a structural feature can include, but is not limited to, a mismatch, a wobble base pair, a symmetric internal loop, an asymmetric internal loop, a symmetric bulge, or an asymmetric bulge. In other instances, a structural feature can be a pre-formed structure (e.g., a GluR2 recruitment hairpin, or a hairpin from U7 snRNA).

As used herein, the term “double-stranded RNA substrate” or “dsRNA substrate” refers to a guide-target RNA scaffold formed upon hybridization of an engineered guide RNA to a target RNA. The resulting double stranded substrate is referred as a “guide target RNA scaffold.” Such guide-target RNA scaffolds can form various secondary, tertiary, and quaternary structures, which may or may not be present in in the gRNA or target RNA prior to hybridization. Accordingly, in some instances, such secondary structures of a guide-target RNA scaffold that are not present in the gRNA prior to hybridization to the target RNA molecule are said to arise from “latent features” of the gRNA molecule. Non-limiting examples of such structural features include mismatches, bulges (e.g., symmetrical bulges or asymmetrical bulges), internal loops (e.g., symmetrical internal loops or asymmetrical internal loops), and hairpins (e.g., recruiting hairpins or a non-recruiting hairpins). Other such structures are further described herein.

In some embodiments, a gRNA described herein has a plurality of structural features, e.g., a combination of latent and actual features. For example, in some embodiments, the gRNA has from 1 to 50 structural features. In some embodiments, the gRNA has from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features. In some embodiments, the plurality of structural features includes one or more latent structures capable of forming a different structural feature of a guide-target RNA scaffold upon hybridization of the gRNA to a target RNA. In some embodiments, the plurality of structural features includes a structural feature formed prior to hybridization of the gRNA to the target RNA, e.g., a GluR2 recruitment hairpin or a hairpin from U7 snRNA.

Similarly, in some embodiments, a guide-target RNA scaffold described herein has a plurality of structural features. For example, in some embodiments, the guide-target RNA scaffold has from 1 to 50 structural features. In some embodiments, the guide-target RNA scaffold has from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features. In some embodiments, the plurality of structural features includes one or more structural features formed, at least in part from a latent structure of the gRNA. In some embodiments, the plurality of structural features includes one or more structural feature formed in the gRNA prior to hybridization to the target RNA, e.g., a GluR2 recruitment hairpin or a hairpin from U7 snRNA. In some embodiments, the plurality of structural features includes one or more structural feature formed in the target RNA prior to hybridization of the gRNA to the target RNA.

As used herein, the term “targeting sequence” can be used interchangeably with “targeting domain” or “targeting region” and refers to a polynucleotide sequence within an engineered guide RNA sequence that is at least partially complementary to a target polynucleotide. The target polynucleotide (e.g., a target RNA or a target DNA) may be a region of a polynucleotide of interest, such as a gene or a messenger RNA. As used herein, a “complementary” sequence refers to a sequence that is a reverse complement relative to a second sequence. A targeting sequence of an engineered guide RNA allows the engineered guide RNA to hybridize to a target polynucleotide (e.g., a target RNA) through base pairing, such as Watson Crick base pairing. A targeting sequence can be located at either the N-terminus or C-terminus of the engineered guide RNA, or both, or the targeting sequence can be within the engineered guide RNA. The targeting sequence can be of any length sufficient to hybridize with the target polynucleotide.

As used herein, the term “target RNA” refers to a ribonucleic acid (RNA) of interest, e.g., for hybridization and/or editing by a deamination enzyme. Target RNA includes, but is not limited to, target messenger RNA (mRNA) (e.g., pre-mRNA and/or mature mRNA), target ribosomal RNA (rRNA), target transfer RNA (tRNA), target small nuclear RNA (snRNA), and the like, including total RNA), which can be present in single- or double-stranded form. Unless otherwise limited, a nucleic acid can comprise known analogs of natural nucleotides, some of which can function in a similar manner as naturally occurring nucleotides.

As used herein, “messenger RNA” or “mRNA” are RNA molecules comprising a sequence that encodes a polypeptide or protein. In general, RNA can be transcribed from DNA. In some cases, precursor mRNA containing non-protein coding regions in the sequence can be transcribed from DNA and then processed to remove all or a portion of the non-coding regions (introns) to produce mature mRNA. As used herein, the term “pre-mRNA” can refer to the RNA molecule transcribed from DNA before undergoing processing to remove the non-protein coding regions.

As used herein, unless otherwise dictated by context “nucleotide” or “nt” refers to ribonucleotide.

As used herein, the terms “patient” and “subject” are used interchangeably, and may be taken to mean any living organism which may be treated with compounds found using the present disclosure. As such, the terms “patient” and “subject” include, but are not limited to, any non-human mammal, primate and human.

The term “stop codon” can refer to a three-nucleotide contiguous sequence within messenger RNA that signals a termination of translation. Non-limiting examples include in RNA, UAG (amber), UAA (ochre), UGA (umber, also known as opal) and in DNA TAG, TAA or TGA. Unless otherwise noted, the term can also include nonsense mutations within DNA or RNA that introduce a premature stop codon, causing any resulting protein to be abnormally shortened.

A “therapeutically effective amount” of a composition is an amount sufficient to achieve a desired therapeutic effect, and does not require cure or complete remission.

The terms “treat,” “treated,” “treating”, or “treatment” as used herein have the meanings commonly understood in the medical arts, and therefore does not require cure or complete remission, and therefore includes any beneficial or desired clinical results. Treatment includes eliciting a clinically significant response without excessive levels of side effects. Treatment also includes prolonging survival as compared to expected survival if not receiving treatment.

As used herein, “preventing” a disease refers to inhibiting the full development of a disease.

As used herein, the term “latent structure” refers to a structural feature that substantially forms only upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed, and the latent structure provided in the guide RNA is, thus, unmasked. The formation and structure of a latent structural feature upon binding to the target RNA depends on the guide RNA sequence. For example, formation and structure of the latent structural feature may depend on a pattern of complementary and mismatched residues in the guide RNA sequence relative to the target RNA. The guide RNA sequence may be engineered to have a latent structural feature that forms upon binding to the target RNA.

As used herein, the term “engineered latent guide RNA” refers to an engineered guide RNA that comprises a portion of sequence that, upon hybridization or only upon hybridization to a target RNA, substantially forms at least a portion of a structural feature, other than a single A/C mismatch feature at the target adenosine to be edited.

As used herein, the term “guide-target RNA scaffold” refers to the resulting double-stranded RNA formed upon hybridization of a guide RNA, with latent structure, to a target RNA. A guide-target RNA scaffold has one or more structural features formed within the double-stranded RNA duplex upon hybridization. For example, the guide-target RNA scaffold can have one or more structural features selected from a bulge, mismatch, internal loop, hairpin, or wobble base pair.

As used herein, the term “structured motif” refers to two or more structural features in a guide-target RNA scaffold.

As used herein, the term “mismatch” refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature. In some embodiments, a mismatch is an A/C mismatch. An A/C mismatch can comprise a C in an engineered guide RNA of the present disclosure opposite an A in a target RNA. An A/C mismatch can comprise an A in an engineered guide RNA of the present disclosure opposite a C in a target RNA. A G/G mismatch can comprise a G in an engineered guide RNA of the present disclosure opposite a G in a target RNA. In some embodiments, a mismatch positioned 5′ of the edit site can facilitate base-flipping of the target A to be edited. A mismatch can also help confer sequence specificity. Thus, a mismatch can be a structural feature formed from latent structure provided by an engineered latent guide RNA.

As used herein, the term “bulge” refers to a structure, substantially formed only upon formation of the guide-target RNA scaffold, where contiguous nucleotides in either the engineered guide RNA or the target RNA are not complementary to their positional counterparts on the opposite strand. A bulge can change the secondary or tertiary structure of the guide-target RNA scaffold. A bulge can have from 0 to 4 contiguous nucleotides on the guide RNA side of the guide-target RNA scaffold and 1 to 4 contiguous nucleotides on the target RNA side of the guide-target RNA scaffold or a bulge can have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a mismatch. Further, where the number of participating nucleotides on either the guide RNA side or the target RNA side exceeds 4, the resulting structure is no longer considered a bulge, but rather, is considered an internal loop.

As used herein, the term “symmetrical bulge” refers to a structure formed when the same number of nucleotides is present on each side of the bulge.

As used herein, the term “asymmetrical bulge” refers to a structure formed when a different number of nucleotides is present on each side of the bulge.

As used herein, the term “internal loop” refers to the structure, substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a bulge or a mismatch, depending on the size of the structural feature. An internal loop can be a symmetrical internal loop or an asymmetrical internal loop.

As used herein, the term “symmetrical internal loop” refers to a structure formed when the same number of nucleotides is present on each side of the internal loop.

As used herein, the term “asymmetrical internal loop” refers to a structure formed when a different number of nucleotides is present on each side of the internal loop.

As used herein, the term “hairpin” refers to an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion.

As used herein, the term “recruitment hairpin” refers to a hairpin structure capable of recruiting, at least in part, an RNA editing entity, such as ADAR. In some cases, a recruitment hairpin can be formed and present in the absence of binding to a target RNA. In some embodiments, a recruitment hairpin is a GluR2 domain or portion thereof. In some embodiments, a recruitment hairpin is an Alu domain or portion thereof. A recruitment hairpin, as defined herein, can include a naturally occurring ADAR substrate or truncations thereof. Thus, a recruitment hairpin such as GluR2 is a pre-formed structural feature that may be present in constructs comprising an engineered guide RNA, not a structural feature formed by latent structure provided in an engineered latent guide RNA.

As used herein, the term “non-recruitment hairpin” refers to a hairpin structure that does not have a primary function of recruiting an RNA editing entity, e.g., that is not capable of recruiting an RNA editing entity. A non-recruitment hairpin, in some instances, does not recruit an RNA editing entity. In some instances, a non-recruitment hairpin has a dissociation constant for binding to an RNA editing entity under physiological conditions that is insufficient for binding. For example, a non-recruitment hairpin has a dissociation constant for binding an RNA editing entity at 25° C. that is greater than about 1 mM, 10 mM, 100 mM, or 1 M, as determined in an in vitro assay. A non-recruitment hairpin can exhibit functionality that improves localization of the engineered guide RNA to the target RNA. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that can be present in constructs comprising engineered guide RNA constructs, not a structural feature formed by latent structure provided in an engineered latent guide RNA.

As used herein, the term “wobble base pair” refers to two bases that weakly base pair. For example, a wobble base pair of the present disclosure can refer to a G paired with a U. Thus, a wobble base pair can be a structural feature formed from latent structure provided by an engineered latent guide RNA.

As used herein, the term “macro-footprint” refers to an over-arching structure of a guide RNA. In some embodiments, a macro-footprint flanks a micro-footprint. Further, while a macro-footprint sequence can flank a micro-footprint sequence, additional latent structures can be incorporated that flank either end of the macro-footprint as well. In some embodiments, such additional latent structures are included as part of the macro-footprint. In some embodiments, such additional latent structures are separate, distinct, or both separate and distinct from the macro-footprint.

As used herein, the term “micro-footprint” refers to a guide structure with latent structures that, when manifested, facilitate editing of the adenosine of a target RNA via an adenosine deaminase enzyme. A macro-footprint can serve to guide an RNA editing entity (e.g., ADAR) and direct its activity towards a micro-footprint. In some embodiments, included within the micro-footprint sequence is a nucleotide that is positioned such that, when the guide RNA is hybridized to the target RNA, the nucleotide opposes the adenosine to be edited by the adenosine deaminase and does not base pair with the adenosine to be edited. This nucleotide is referred to herein as the “mismatched position” or “mismatch” and can be a cytosine. Micro-footprint sequences as described herein have upon hybridization of the engineered guide RNA and target RNA, at least one structural feature selected from the group consisting of: a bulge, an internal loop, a mismatch, a hairpin, and any combination thereof. Engineered guide RNAs with superior micro-footprint sequences can be selected based on their ability to facilitate editing of a specific target RNA. Engineered guide RNAs selected for their ability to facilitate editing of a specific target are capable of adopting various micro-footprint latent structures, which can vary on a target-by-target basis.

As used herein, the term “barbell” refers to a guide macro-footprint having a pair of internal loop latent structures that manifest upon hybridization of the guide RNA to the target RNA.

As used herein, the term “dumbbell” refers to a macro-footprint having two symmetrical internal loops, wherein the target A to be edited is positioned between the two symmetrical loops for selective editing of the target A. The two symmetrical internal loops are each formed by 6 nucleotides on the guide RNA side of the guide-target RNA scaffold and 6 nucleotides on the target RNA side of the guide-target RNA scaffold. Thus, a dumbbell can be a structural feature formed from latent structure provided by an engineered latent guide RNA.

As used herein, the term “U-deletion” refers to a type of asymmetrical bulge. In some embodiments, a U-deletion is an asymmetrical bulge formed upon binding of an engineered guide RNA to an mRNA transcribed from a target gene. In some embodiments, a U-deletion is formed by 0 nucleotides on the engineered guide RNA side of the guide-target RNA scaffold and 1 nucleotide on the target RNA side of the guide-target RNA scaffold. For instance, in some implementations, a U-deletion is formed by an “A” on the target RNA side of the guide-target RNA scaffold and a deletion of a “U” on the engineered guide RNA side of the guide-target RNA scaffold. In some embodiments, U-deletions are used opposite of a local off-target nucleotide position (e.g., an off-target adenosine) to reduces off-target editing.

As used herein, the term “base paired region” or “bp region” refers to a region of the guide-target RNA scaffold in which bases in the guide RNA are paired with opposing bases in the target RNA. Base paired regions can extend from one end or proximal to one end of the guide-target RNA scaffold to or proximal to the other end of the guide-target RNA scaffold. Base paired regions can extend between two structural features. Base paired regions can extend from one end or proximal to one end of the guide-target RNA scaffold to or proximal to a structural feature. Base paired regions can extend between two structural features. Base paired regions can extend from one end or proximal to one end of the guide-target RNA scaffold to or proximal to a structural feature. Base paired regions can extend from a structural feature to the other end of the guide-target RNA scaffold.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

As used herein, “tropism” of a recombinant adeno-associated virus (rAAV), such as a rAAV5, for a tissue refers to the ability of a given rAAV to preferentially infect a given cell type or tissue. A degree of tropism may be determined by a ratio of an infection rate in a targeted tissue to an infection rate in a different, non-targeted tissue. As used herein, increased tropism for a given cell type or tissue, such as increased tropism conferred by a 581-589 region, is determined relative to a wild type AAV5 capsid. As used herein, “detargeting” of a rAAV to a tissue may refer to the ability of a given rAAV to avoid infecting a detargeted tissue or cell type while infecting one or more other tissues or cell types. A degree of detargeting may be determined by a ratio of an infection rate in a detargeted tissue to an infection rate of a different, non-detargeted tissue. As used herein, increased detargeting for a given cell type or tissue, such as increased detargeting conferred by a 581-589 region, is determined relative to a wild type AAV5 capsid.

As used herein, “tissue tropism” refers to a preference of a virus having an engineered VP capsid polypeptide of the present disclosure to infect a given tissue or be enriched in or accumulate in a given tissue. A “tissue-tropic” rAAV may specifically target or infect a first tissue or set of tissues (e.g., CNS, cardiac, liver, skeletal muscle, skin, bone, eye, and/or other tissues) and may not target or infect a second tissue or set of tissues. Alternatively or additionally, in some embodiments, a respective tissue target includes, but is not limited to, adrenal gland, aorta, bone with bone marrow, brain (cerebellum), brain (hippocampus, dentate gyrus), brain (hippocampus, CA1), brain (hippocampus, CA3), brain (hypothalamus), brain (cortex, temporal), brain (cortex, forebrain), brain (cortex, occipital), brain (substantia nigra), brain (thalamus), cecum, colon, duodenum, epididymis, esophagus, eye, gallbladder, heart, ileum, jejunum, kidney, liver, lung, lymph node(s), mammary gland, ovary, pancreas, parathyroid gland, peripheral nerve (sciatic), pituitary, prostate, salivary gland, seminal vesicle, skeletal muscle, skin, spinal cord, spleen, stomach, testis, thymus, thyroid gland, trachea, urinary bladder, uterus, and vagina. For example, a “CNS-tropic” rAAV may specifically target or infect CNS tissue and may not target or infect liver, muscle, skin, bone, eye, or other tissues. In another example a “CNS and cardiac-tropic” rAAV may specifically target or infect CNS and cardiac tissues and may not target or infect liver, skeletal muscle, skin, bone, eye, or other tissues. A “tissue-detargeted” rAAV may specifically avoid targeting or avoid infection of the detargeted tissue or set of tissues while infecting a second tissue or set of tissues. For example, a “liver-detargeted” rAAV may not target or infect liver tissue but may infect one or more other tissues, such as CNS or cardiac tissue. Tissue tropism or tissue detargeting, when used as a relative term and depending on the context in which it is described herein, refers to an increase or decrease in tissue tropism of a given rAAV virion having a first capsid polypeptide in a first tissue as compared to a second tissue and/or refers to an increase or decrease in tissue tropism of a given rAAV virion having a first capsid polypeptide to an rAAV virion having a second capsid polypeptide. In some embodiments, the first tissue can be a group of tissues. In some embodiments, the second tissue can be a group of tissues. For example, the first tissue may be CNS or cardiac tissues and the second tissue may be a non-CNS or non-cardiac tissue consisting collectively of kidney, liver, skeletal muscle, lung, spleen, lymph node, bone marrow, mammary gland, skin, adrenal gland, thyroid, colon, sciatic nerve, and spinal cord tissues.

As used herein, the term “VP” refers to a viral capsid protein. For simplicity throughout this disclosure, viral capsid protein is generally referred to as “VP.” Viral capsid protein is referred to as VP1 when referencing AAV5 VP1 positional notation. In all cases, viral capsid sequences and mutations disclosed herein should be understood as pertaining to all isoforms of the capsid protein (VP1, VP2, and VP3), as a mixture of these isoforms assemble to form virions. The positional amino acid residue designations “581 to 589” are relative to the translational start of the VP1 polypeptide and should be adjusted accordingly to the relative start sites of VP2 and VP3. It should be understood that the present disclosure, when describing any particular VP1 sequence with mutations at particular amino acid residue positions, necessarily also encompasses corresponding mutations in VP2 and VP3. For example, any consensus sequence or specific sequence of a VP1 capsid protein having one or more mutations in the 581-589 region, corresponding to amino acid residues 581 to 589 of VP1, also encompasses VP2 and VP3 capsid proteins having said one or more mutations in an amino acid residue region in VP2 and VP3 corresponding to the amino acid residues of the VP1 581 to 589 region.

As used herein, “581-589 region” refers to a region or fragment of VP1 corresponding to amino acid residues 581 to 589 relative to the translational start of the VP1 polypeptide. The 581-589 region corresponds to amino acid residues 445 to 453 of VP2 and to amino acid residues 389 to 397 of VP3. The 581-589 region may confer tissue tropism to an AAV, and defined variants may be engineered to confer tissue tropism to an rAAV formed from viral capsid polypeptides (VP1, VP2, and VP3) comprising the 581-589 region.

It should be understood that the present disclosure includes polynucleotide sequences encoding for any sequence disclosed herein. For example, if an amino acid sequence is provided, the present disclosure also encompasses a polynucleotide sequence encoding for said amino acid sequence. It should be understood that further embodiments include mutations in VP1, VP2, VP3, or any combination thereof that do not alter the desired properties (e.g., a particular tissue tropism) or affect viral assembly, as described herein. In some embodiments, an rAAV virion is made of a capsid that may include the engineered AAV5 VP capsid polypeptides disclosed herein (e.g., engineered VP1, VP2, and VP3 capsid polypeptides comprising a variant 581-589 region).

As used herein, the term “model” refers to a machine learning model or algorithm.

In some embodiments, a model includes an unsupervised learning algorithm. One example of an unsupervised learning algorithm is cluster analysis. In some embodiments, a model includes a supervised machine learning algorithm. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, Gradient Boosting, mixture models, hidden Markov models, Gaussian NB algorithms, linear discriminant analysis, or any combinations thereof. In some embodiments, a model is a multinomial classifier algorithm. In some embodiments, a model is a 2-stage stochastic gradient descent (SGD) model. In some embodiments, a model is a deep neural network (e.g., a deep-and-wide sample-level model).

Neural networks. In some embodiments, the model is a neural network (e.g., a convolutional neural network and/or a residual neural network). Neural network algorithms, also known as artificial neural networks (ANNs), include convolutional and/or residual neural network algorithms (deep learning algorithms). In some embodiments, neural networks are machine learning algorithms that are trained to map an input dataset to an output dataset, where the neural network includes an interconnected group of nodes organized into multiple layers of nodes. For example, in some embodiments, the neural network architecture includes at least an input layer, one or more hidden layers, and an output layer. In some embodiments, the neural network includes any total number of layers, and any number of hidden layers, where the hidden layers function as trainable feature extractors that allow mapping of a set of input data to an output value or set of output values. In some embodiments, a deep learning algorithm is a neural network including a plurality of hidden layers, e.g., two or more hidden layers. In some instances, each layer of the neural network includes a number of nodes (or “neurons”). In some embodiments, a node receives input that comes either directly from the input data or the output of nodes in previous layers, and performs a specific operation, e.g., a summation operation. In some embodiments, a connection from an input to a node is associated with a parameter (e.g., a weight and/or weighting factor). In some embodiments, the node sums up the products of all pairs of inputs, xi, and their associated parameters. In some embodiments, the weighted sum is offset with a bias, b. In some embodiments, the output of a node or neuron is gated using a threshold or activation function, f, which, in some instances, is a linear or non-linear function. In some embodiments, the activation function is, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

In some implementations, the weighting factors, bias values, and threshold values, or other computational parameters of the neural network, are “taught” or “learned” in a training phase using one or more sets of training data. For example, in some implementations, the parameters are trained using the input data from a training dataset and a gradient descent, for example, back-propagation, method so that the output value(s) that the ANN computes are consistent with the examples included in the training dataset. In some embodiments, the parameters are obtained from a back propagation neural network training process.

Any of a variety of neural networks are suitable for use in accordance with the present disclosure. Examples include, but are not limited to, feed-forward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. In some implementations, convolutional and/or residual neural networks are used, in accordance with the present disclosure.

For instance, a deep neural network model includes an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 50 parameters, at least 100 parameters, at least 1000 parameters, at least 2000 parameters, at least 5000 parameters, at least 1×10⁴ parameters, at least 1×10⁵ parameters, at least 1×10⁶ parameters, at least 1×10⁷ parameters, or at least 1×10⁸ parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

Support vector machines. In some embodiments, the model is a support vector machine (SVM). SVM algorithms suitable for use as models are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For certain cases in which no linear separation is possible, SVMs work in combination with the technique of ‘kernels’, which automatically realizes a non-linear mapping to a feature space. The hyper-plane found by the SVM in feature space corresponds, in some instances, to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM model requires a computer to calculate because it cannot be mentally solved.

Naïve Bayes algorithms. In some embodiments, the model is a Naive Bayes algorithm. Naïve Bayes models suitable for use as models are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes model is any model in a family of “probabilistic models” based on applying Bayes' theorem with strong (naive) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor algorithms. In some embodiments, a model is a nearest neighbor algorithm. In some implementations, nearest neighbor models are memory-based and include no model to be fit. For nearest neighbors, given a query point x₀ (a test subject), the k training points x₍ᵣ₎, r, . . . , k (here the training subjects) closest in distance to x₀ are identified and then the point x₀ is classified using the k nearest neighbors. In some embodiments, Euclidean distance in feature space is used to determine distance as d₍ᵢ₎=∥x₍ᵢ₎−x₍₀₎∥. Typically, when the nearest neighbor algorithm is used, the abundance data used to compute the linear discriminant is standardized to have mean zero and variance 1. In some embodiments, the nearest neighbor rule is refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor model is a non-parametric machine learning method in which the input consists of the k closest training examples in feature space. In some embodiments, the output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the k-nearest neighbor model is used for regression and the output is a prediction of a property value of the object determined as an average of the values of the k nearest neighbors. In some embodiments, the number of distance calculations needed to solve the k-nearest neighbor model is such that a computer is used to solve the model for a given input because it cannot be mentally performed.

Random forest, decision tree, and boosted tree algorithms. In some embodiments, the model is a decision tree. Decision trees suitable for use as models are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. For example, one specific algorithm is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree model includes at least 10, at least 20, at least 50, or at least 100 parameters (e.g., weights and/or decisions) and requires a computer to calculate because it cannot be mentally solved.

Regression. In some embodiments, the model uses a regression algorithm. In some embodiments, a regression algorithm is any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the model. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the model makes use of a regression model disclosed in Hastie et al., 2001, The Elements of Statistical Learning, Springer-Verlag, New York. In some embodiments, the logistic regression model includes at least 10, at least 20, at least 50, at least 100, or at least 1000 parameters (e.g., weights) and requires a computer to calculate because it cannot be mentally solved.

Linear discriminant analysis algorithms. In some embodiments, linear discriminant analysis (LDA), normal discriminant analysis (NDA), or discriminant function analysis is a generalization of Fisher's linear discriminant, a method used in statistics, pattern recognition, and machine learning to find a linear combination of features that characterizes or separates two or more classes of objects or events. In some embodiments, the resulting combination is used as the model (linear model) in some embodiments of the present disclosure.

Mixture model and Hidden Markov model. In some embodiments, the model is a mixture model, such as that described in McLachlan et al., Bioinformatics 18(3):413-422, 2002. In some embodiments, in particular, those embodiments including a temporal component, the model is a hidden Markov model such as described by Schliep et al., 2003, Bioinformatics 19(1):i255-i263.

Clustering. In some embodiments, the model is an unsupervised clustering model. In some embodiments, the model is a supervised clustering model. Clustering algorithms suitable for use as models are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. As an illustrative example, in some embodiments, the clustering problem is described as one of finding natural groupings in a dataset. To identify natural groupings, two issues are addressed. First, a way to measure similarity (or dissimilarity) between two samples is determined. This metric (e.g., similarity measure) is used to ensure that the samples in one cluster are more like one another than they are to samples in other clusters. Second, a mechanism for partitioning the data into clusters using the similarity measure is determined. One way to begin a clustering investigation is to define a distance function and to compute the matrix of distances between all pairs of samples in the training set. If distance is a good measure of similarity, then the distance between reference entities in the same cluster is significantly less than the distance between the reference entities in different clusters. However, in some implementations, clustering does not use a distance metric. For example, in some embodiments, a nonmetric similarity function s(x, x′) is used to compare two vectors x and x′. In some such embodiments, s(x, x′) is a symmetric function whose value is large when x and x′ are somehow “similar.” Once a method for measuring “similarity” or “dissimilarity” between points in a dataset has been selected, clustering uses a criterion function that measures the clustering quality of any partition of the data. Partitions of the dataset that extremize the criterion function are used to cluster the data. Particular exemplary clustering techniques contemplated for use in the present disclosure include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering includes unsupervised clustering (e.g., with no preconceived number of clusters and/or no predetermination of cluster assignments).

Ensembles of models and boosting. In some embodiments, an ensemble (two or more) of models is used. In some embodiments, a boosting technique such as AdaBoost is used in conjunction with many other types of learning algorithms to improve the performance of the model. In this approach, the output of any of the models disclosed herein, or their equivalents, is combined into a weighted sum that represents the final output of the boosted model. In some embodiments, the plurality of outputs from the models is combined using any measure of central tendency known in the art, including but not limited to a mean, median, mode, a weighted mean, weighted median, weighted mode, etc. In some embodiments, the plurality of outputs is combined using a voting method. In some embodiments, a respective model in the ensemble of models is weighted or unweighted.

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n 50; n≥75; n≥100; n≥125; n≥150; n 200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, n≥1×10⁷, n≥1×10⁸, or ≥1×10⁹. In some embodiments, the plurality of parameters comprises no more than 1×10¹⁰, no more than 1×10⁹, no more than 1×10⁸, no more than 1×10⁷, no more than 1×10⁶, no more than 1×10⁵, no more than 1×10⁴, or no more than 1×10³. As such, the algorithms, models, regressors, and/or classifiers of the present disclosure cannot be mentally performed. In some embodiments n is between 10,000 and 1×10⁷, between 100,000 and 5×10⁶, or between 500,000 and 1×10⁶. In some embodiments, the plurality of parameters falls within another range starting no lower than 2 parameters and ending no higher than 1×10¹⁰ parameters. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

As used herein, the term “untrained model” refers to a machine learning model or algorithm, such as a classifier or a neural network, that has not been trained on a target dataset. In some embodiments, “training a model” (e.g., “training a neural network”) refers to the process of training an untrained or partially trained model (e.g., “an untrained or partially trained neural network”). Moreover, it will be appreciated that the term “untrained model” does not exclude the possibility that transfer learning techniques are used in such training of the untrained or partially trained model. For instance, Fernandes et al., 2017, “Transfer Learning with Partial Observability Applied to Cervical Cancer Screening,” Pattern Recognition and Image Analysis: 8^{th} Iberian Conference Proceedings, 243-250, which is hereby incorporated by reference, provides non-limiting examples of such transfer learning. In instances where transfer learning is used, the untrained model described above is provided with additional data over and beyond that of the primary training dataset. Typically, this additional data is in the form of parameters (e.g., coefficients, weights, and/or hyperparameters) that were learned from another, auxiliary training dataset. Moreover, while a description of a single auxiliary training dataset has been disclosed, it will be appreciated that there is no limit on the number of auxiliary training datasets that can be used to complement the primary training dataset in training the untrained model in the present disclosure. For instance, in some embodiments, two or more auxiliary training datasets, three or more auxiliary training datasets, four or more auxiliary training datasets or five or more auxiliary training datasets are used to complement the primary training dataset through transfer learning, where each such auxiliary dataset is different than the primary training dataset. Any manner of transfer learning is used, in some such embodiments. For instance, consider the case where there is a first auxiliary training dataset and a second auxiliary training dataset in addition to the primary training dataset. In such a case, the parameters learned from the first auxiliary training dataset (by application of a first model to the first auxiliary training dataset) are applied to the second auxiliary training dataset using transfer learning techniques (e.g., a second model that is the same or different from the first model), which in turn results in a trained intermediate model whose parameters are then applied to the primary training dataset and this, in conjunction with the primary training dataset itself, is applied to the untrained model. Alternatively, in another example embodiment, a first set of parameters learned from the first auxiliary training dataset (by application of a first model to the first auxiliary training dataset) and a second set of parameters learned from the second auxiliary training dataset (by application of a second model that is the same or different from the first model to the second auxiliary training dataset) are each individually applied to a separate instance of the primary training dataset (e.g., by separate independent matrix multiplications) and both such applications of the parameters to separate instances of the primary training dataset in conjunction with the primary training dataset itself (or some reduced form of the primary training dataset such as principal components or regression coefficients learned from the primary training set) are then applied to the untrained model in order to train the untrained model.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, and components have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For instance, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The present description includes example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The present description, for purpose of explanation, is described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. Where particular values are described in the application and claims, unless otherwise stated, the term “about” means within an acceptable error range for the particular value. The term “about” can have the meaning as commonly understood by one of ordinary skill in the art. The term “about” can refer to +10%. The term “about” can refer to +5%.

### 6.3. Example System Embodiments

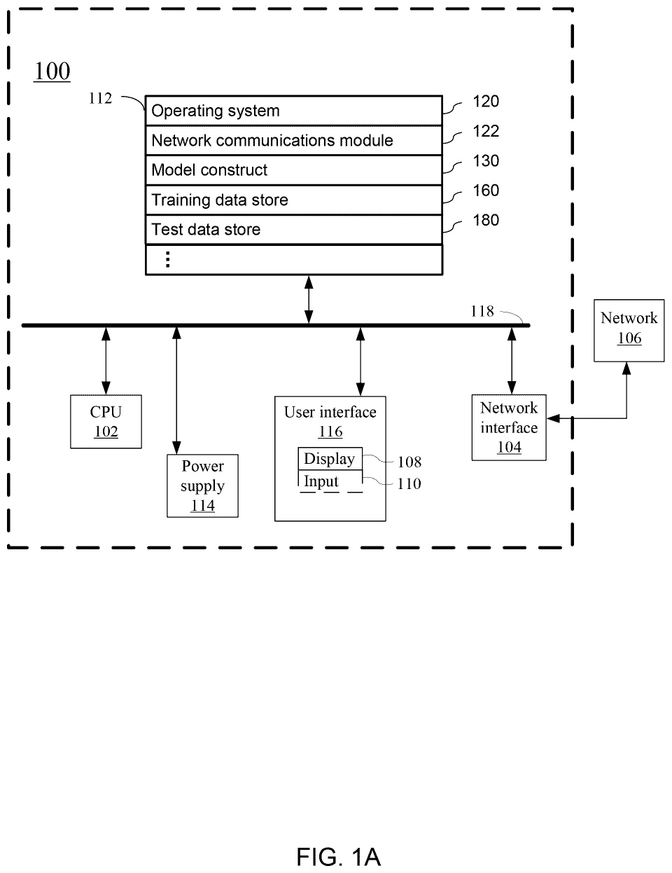

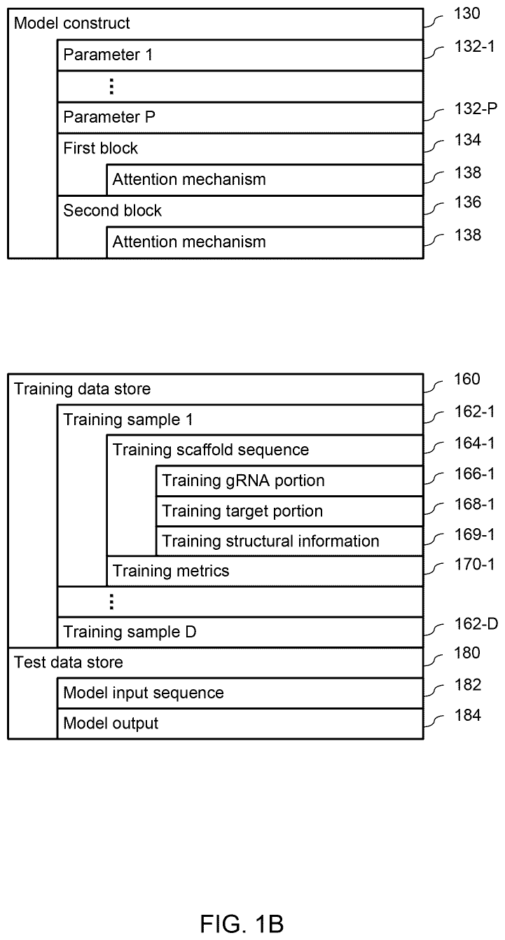

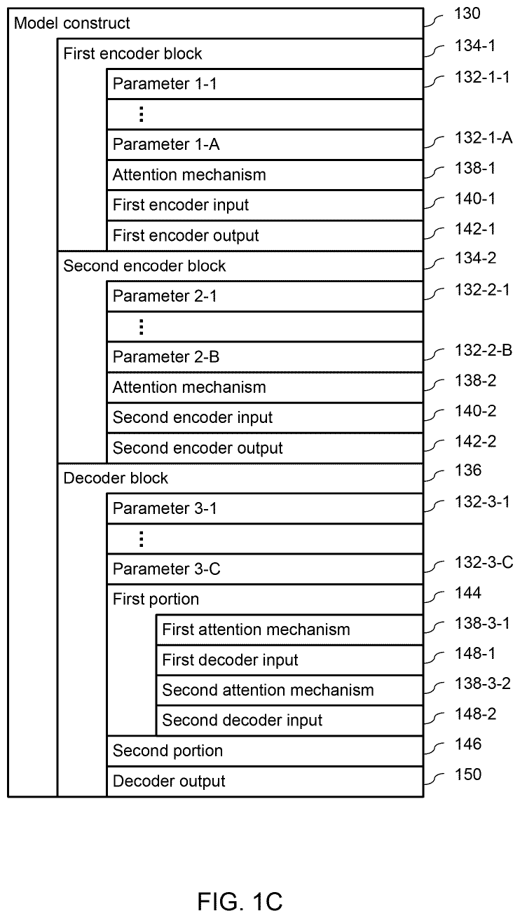

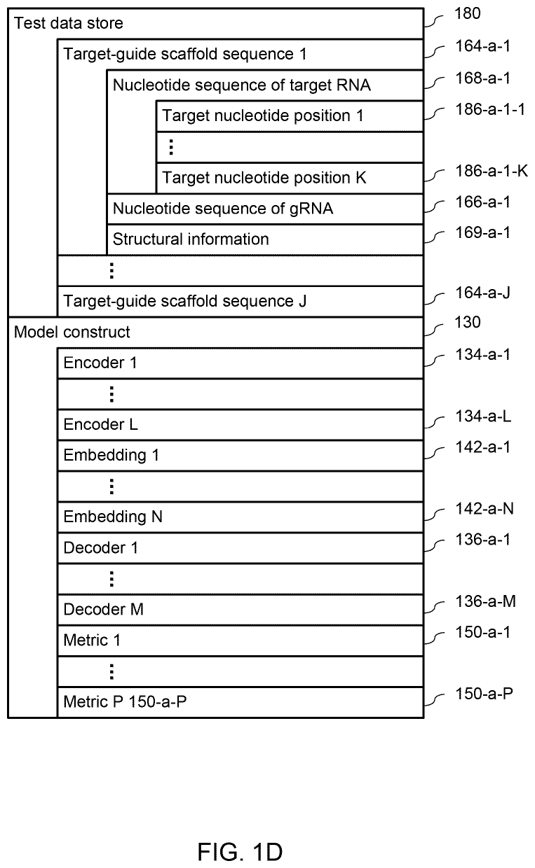

In the present disclosure, unless expressly stated otherwise, descriptions of devices and systems will include implementations of one or more computers. For instance, and for purposes of illustration in FIGS. 1A-D, a computer system 100 is represented as single device that includes all the functionality of the computer system 100. However, the present disclosure is not limited thereto. For instance, in some embodiments, the functionality of the computer system 100 is spread across any number of networked computers and/or reside on each of several networked computers and/or by hosted on one or more virtual machines and/or containers at a remote location accessible across a communications network (e.g., communications network 106). One of skill in the art will appreciate that a wide array of different computer topologies is possible for the computer system 100, and other devices and systems of the preset disclosure, and that all such topologies are within the scope of the present disclosure. Moreover, rather than relying on a physical communications network 106, the illustrated devices and systems may wirelessly transmit information between each other. As such, the exemplary topology shown in FIGS. 1A-D merely serves to describe the features of an embodiment of the present disclosure in a manner that will be readily understood to one of skill in the art.

FIG. 1A depicts a block diagram of a computer system 100 according to some embodiments of the present disclosure. The computer system 100 at least facilitates predicting a deamination efficiency or specificity and/or generating a candidate sequence for a guide RNA (gRNA).

In some embodiments, the prediction of the deamination efficiency or specificity and/or the generation of the candidate sequence for the gRNA is prepared at the computer system 100. In some embodiments, the prediction of the deamination efficiency or specificity and/or the generation of the candidate sequence for the gRNA is then provided (e.g., communicated through communication network 106) to a subject through a display of a respective client device. However, the present disclosure is not limited thereto.

In some embodiments, the communication network 106 optionally includes the Internet, one or more local area networks (LANs), one or more wide area networks (WANs), other types of networks, or a combination of such networks.

Examples of communication networks 106 include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

In various embodiments, the computer system 100 includes one or more processing units (CPUs, processing cores, etc.) 102, a network or other communications interface 104, and memory 112. In some embodiments, the computer system 100 includes a power supply 114 configured to provide a current to one or more components and/or hardware devices of the computer system 100 or a remote device.

In some embodiments, the computer system 100 includes a user interface 116. The user interface 116 typically includes a display 108 for presenting media, such as an output from a model of the present disclosure. In some embodiments, the display 108 is integrated within the computer system (e.g., housed in the same chassis as the CPU 102 and memory 112). In some embodiments, the computer system 100 includes one or more input device(s) 110, which allow a subject to interact with the computer system 100. In some embodiments, the one or more input devices 110 include a keyboard, a mouse, and/or other input mechanisms. Alternatively, or in addition, in some embodiments, the display 108 includes a touch-sensitive surface (e.g., where display 108 is a touch-sensitive display or computer system 100 includes a touch pad).

In some embodiments, the computer system 100 presents media to a user through the display 108. Examples of media presented by the display 108 include a prediction of a deamination efficiency or specificity, a generation of the candidate sequence for the gRNA, an output from a model, or a combination thereof. In typical embodiments, the media is presented by the display 108 through a client application.

In some embodiments, memory 112 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. In some embodiments, memory 112 optionally includes one or more storage devices remotely located from the CPU(s) 102. In some embodiments, memory 112, or alternatively the non-volatile memory device(s) within memory 112, includes a non-transitory computer readable storage medium. Access to memory 112 by other components of the computer system 100, such as the CPU(s) 102, is, optionally, controlled by a controller. In some embodiments, the memory 112 include mass storage that is remotely located with respect to the CPU(s) 102. In other words, some data stored in memory 112 is in fact hosted on devices that are external to the computer system 100, but that can be electronically accessed by the computer system 100 over an Internet, intranet, or other form of network 106 or electronic cable using communication interface 104.

In some embodiments, the memory 112 of the computer system 100 for predicting a deamination efficiency or specificity and/or generating a candidate sequence for a gRNA stores:
an optional operating system 120 (e.g., ANDROID, iOS, DARWIN, RTXC, LINUX, UNIX, OS X, WINDOWS, or an embedded operating system such as VxWorks) that includes procedures for handling various basic system services;
an optional network communications module 122 associated with the computer system 100 that identifies the computer system 100 (e.g., within the communication network 106);
an optional model construct 130;
an optional training data store 160; and
an optional test data store 180.

In some embodiments, the computer system 100 includes an operating system 120 that includes procedures for handling various basic system services. The operating system 120 (e.g., iOS, DARWIN, RTXC, LINUX, UNIX, OS X, WINDOWS, or an embedded operating system such as VxWorks) includes various software components and/or drivers for controlling and managing general system tasks (e.g., memory management, storage device control, power management, etc.) and facilitates communication between various hardware and software components of the computer system.

In some embodiments, an optional network communications module 122 is associated with the computer system 100. The optional network communications module 122 is utilized to, at least, uniquely identify the computer system 100 from other devices and components (e.g., uniquely identify computer system 100 from a first client device, etc.). For instance, in some embodiments, the optional network communications module is utilized to receive information from a client device.

Referring to FIG. 1B, in some embodiments, the system 100 at least includes instructions for predicting a deamination efficiency or specificity, and/or optimizing a model to predict the same, in accordance with some embodiments of the present disclosure.

In some embodiments, the optional model construct 130 comprises a plurality of parameters 132 (e.g., 132-1, . . . 132-P) across a first block 134 and a second block 136, where each of the first block 134 and the second block 136 comprises an attention mechanism 138, and the plurality of parameters reflects, at least in part, pretraining information for a plurality of pretraining samples comprising, for each respective pretraining sample in the plurality of pretraining samples, a corresponding unlabeled nucleic acid sequence. In some embodiments, the optional test data store 180 comprises, as input 182 to the model, first test information comprising a respective nucleic acid sequence and, as output 184 from the model, an indication of a structure or function associated with the nucleic acid sequence 182, or a representation thereof. In some embodiments, the optional training data store 160 comprises a plurality of training samples 162 (e.g., 162-1, . . . 162-D), where each respective training sample in the plurality of training samples comprises training information comprising (i) a corresponding training nucleic acid sequence 164 (e.g., 164-1) for a guide RNA (gRNA)-target RNA scaffold formed between the gRNA 166 (e.g., 166-1) and the target RNA 168 (e.g., 168-1) when the gRNA hybridizes to the target RNA, and (ii) a corresponding training set of one or more metrics 170 (e.g., 170-1) for an efficiency or specificity of deamination of a target nucleotide position in the target RNA by an Adenosine Deaminase Acting on RNA (ADAR) protein when facilitated by hybridization of the gRNA 166 to the target RNA 168; thereby updating the plurality of parameters 132. Optionally, the training information further includes corresponding training structural information 169 (e.g., 169-1) about the gRNA-target RNA scaffold.

Referring to FIG. 1C, alternatively or additionally, in some embodiments, the optional model construct 130 comprises a first encoder block 134-1, a second encoder block 134-2, and a decoder block 136.

In some embodiments, the first encoder block 134-1 comprises a first set of parameters 132-1 (e.g., 132-1-1, . . . 132-1-A), in a plurality of parameters of the model. In some embodiments, the first encoder block 134-1 comprises an attention mechanism 138 (e.g., 138-1). In some embodiments, the first encoder block 134-1 (i) receives, as input 140-1, a first portion 166 of the nucleic acid sequence 182 for the gRNA-target RNA scaffold that corresponds to a sequence of the gRNA, or a representation thereof, and (ii) generates, as output 142-1, a representation of the first portion of the nucleic acid sequence.

In some embodiments, the second encoder block 134-2 comprises a second set of parameters 132-2 (e.g., 132-2-1, . . . 132-2-B), in a plurality of parameters of the model. In some embodiments, the second encoder block 134-2 comprises an attention mechanism 138 (e.g., 138-2). In some embodiments, the second encoder block 134-2 (i) receives, as input 140-2, a second portion 168 of the nucleic acid sequence 182 for the gRNA-target RNA scaffold that corresponds to a sequence of the target RNA, or a representation thereof, and (ii) generates, as output 142-2, a representation of the second portion of the nucleic acid sequence.

In some embodiments, the decoder block 136 comprises a third set of parameters 132-3 (e.g., 132-3-1, . . . 132-3-C), in the plurality of parameters of the model. In some embodiments, the decoder block 136 comprises a first portion 144 and a second portion 146, wherein the first portion 144 comprises a first attention mechanism 138 (e.g., 138-3-1) that receives, as input 148-1, the output from the first encoder block 142-1 and a second attention mechanism 138 (e.g., 138-3-2) that receives, as input 148-2, the output from the second encoder block 142-2. In some embodiments, the decoder block includes an output 150 that comprises, as output 184 from the model 130, a predicted set of one or more metrics for the efficiency or specificity of deamination of the target nucleotide position in the target RNA by the ADAR protein when facilitated by hybridization of the test gRNA to the target RNA.

Referring to FIG. 1D, alternatively or additionally, in some embodiments, the present disclosure provides systems for predicting a deamination efficiency or specificity at one or more target nucleotide positions 186 (e.g., 186-a-1-1, . . . 186-a-1-K) of a target RNA. In some embodiments, the system includes at least one processor and a memory storing at least one program for execution by the at least one processor. In some embodiments, the system includes a test data store including information about a target-guide scaffold 164 (e.g., 164-a-1, . . . 164-a-J) formed between a guide RNA (gRNA) and the target RNA when the gRNA hybridizes to the target RNA, where the information comprises a nucleotide sequence of the gRNA 166 (e.g., 166-a-1), a nucleotide sequence of the target RNA 168 (e.g., 168-a-1) comprising the one or more target nucleotide positions 186, and structural information 169 (e.g., 169-a-1) about the target-guide scaffold. In some embodiments, the system includes a model construct 130, including a first portion comprising one or more encoder blocks 134 (e.g., 134-a-1, . . . 134-a-L) that attend to a representation of the nucleotide sequence of the gRNA 166, a representation of the nucleotide sequence of the target RNA 168, and a representation of the structural information 169 about the target-guide scaffold to generate one or more embeddings 142 (e.g., 142-a-1, . . . 142-a-N), and a second portion comprising one or more decoder blocks 136 (e.g., 136-a-1, . . . 136-a-M) that attend to the one or more embeddings 142 to generate the predicted set of one or more metrics 150. In some embodiments, responsive to inputting the information into the model construct 130, the model generates, as output 184, a predicted set of one or more metrics 150 (e.g., 150-a-1, . . . 150-a-P) for an efficiency or specificity of deamination of the one or more target nucleotide positions 186 in the target RNA by a deamination enzyme when facilitated by hybridization of the gRNA to the target RNA.

### 6.4. Example Methods for Prediction of Deamination Efficiency or Specificity

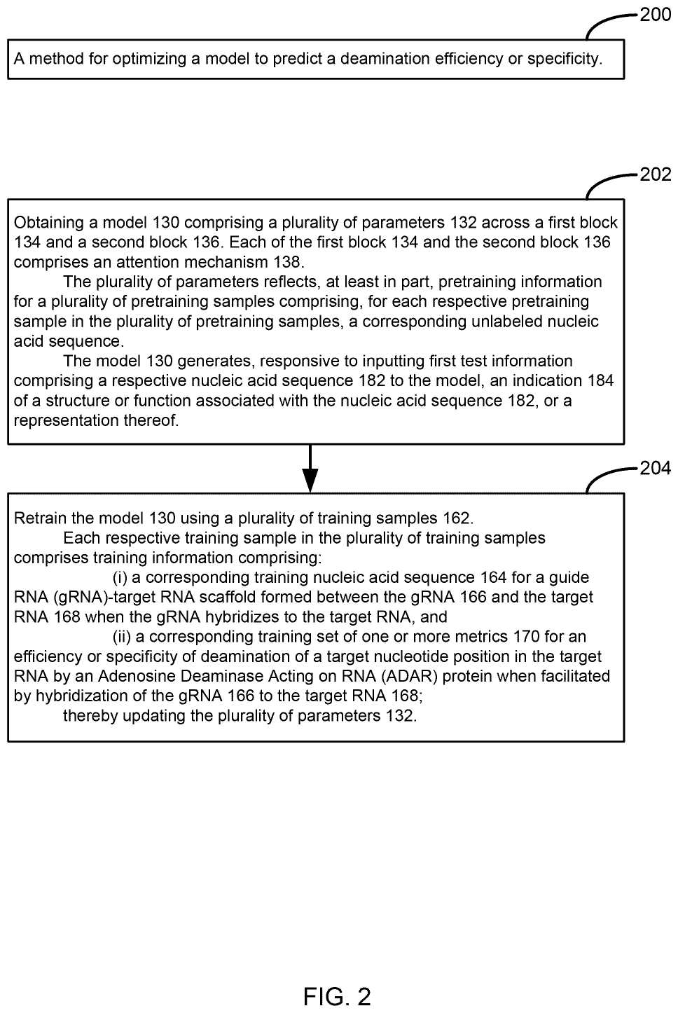

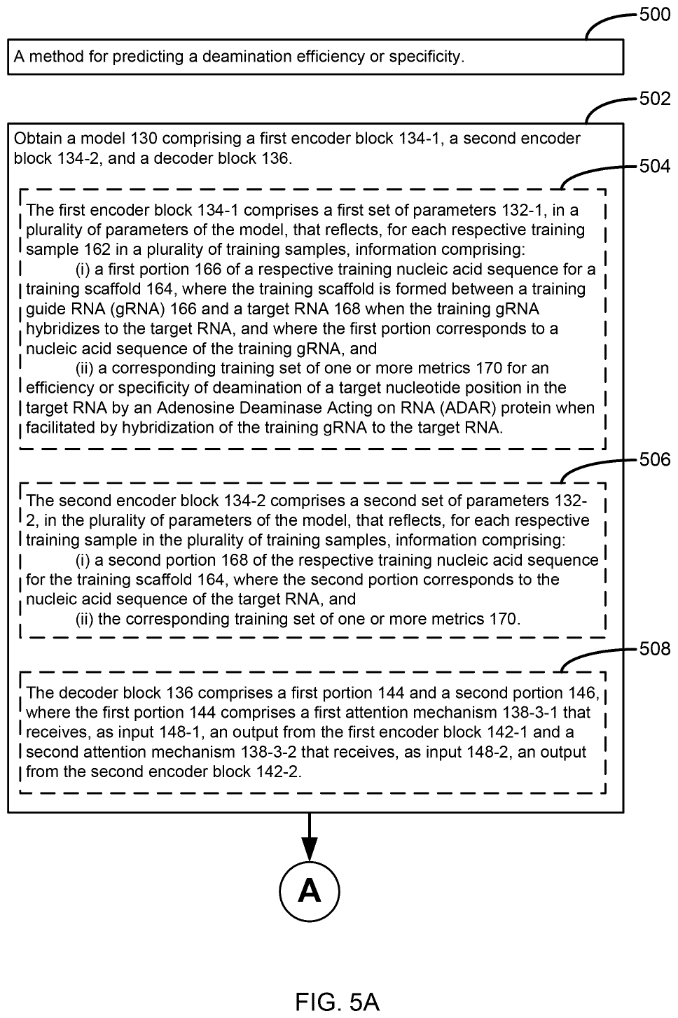

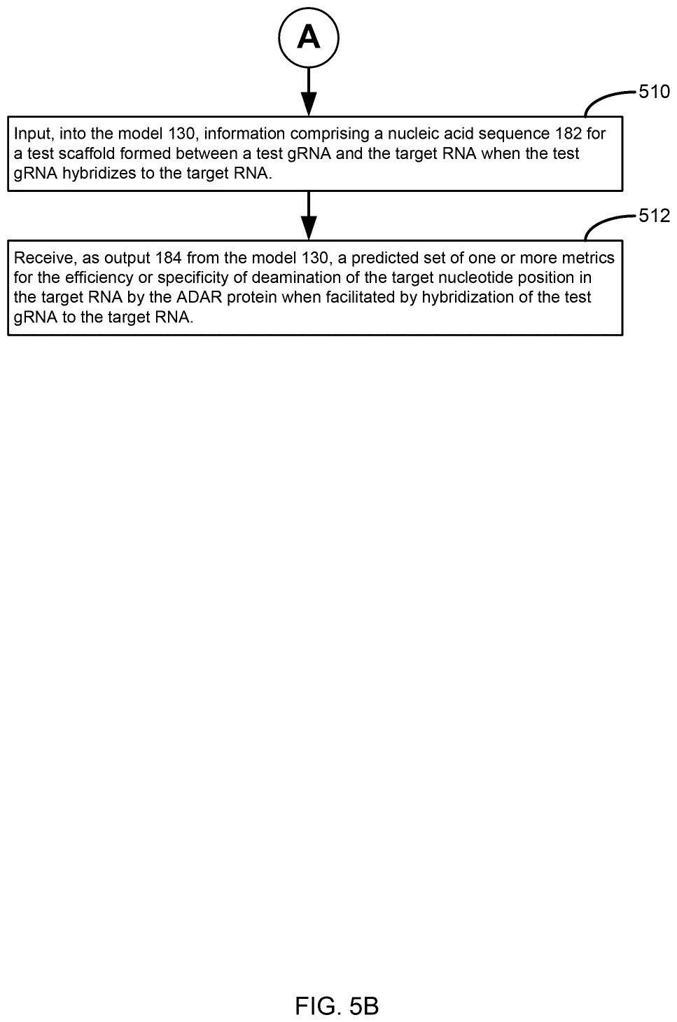

Now that a general topology of a system 100 has been described in accordance with various embodiments of the present disclosures, details regarding some processes in accordance with FIGS. 2, 5A-B, 6, and 13A-B will be described. FIGS. 2, 5A-B, 6, and 13A-B illustrate example flowcharts of methods (e.g., methods 200, 500, 600, and 1300) for predicting a deamination efficiency or specificity and/or optimizing a model to predict the same, in accordance with embodiments of the present disclosure. Various modules in a memory of a computer system and/or a memory of a client device perform certain processes of the methods described in FIGS. 2, 5A-B, 6, and 13A-B, unless expressly stated otherwise. Furthermore, it will be appreciated that the processes in FIGS. 2, 5A-B, 6, and 13A-B can be encoded in a single module or any combination of modules.

Described herein, among other aspects, are applications of machine learning, that may optionally be coupled with high throughput screening (HTS), for the prediction, identification, and/or de novo generation of gRNA capable of facilitating ADAR or APOBEC-mediated editing of one or more target nucleotide sites in an RNA molecule. These approaches allow for the exploration of the enormous gRNA design space to propose highly efficient and specific novel gRNA designs that can be validated experimentally.

In some embodiments, the disclosure describes systems and methods capable of assessing many structurally unique gRNAs (e.g., hundreds of thousands, millions, billions, or more gRNA sequences) against any target sequence, e.g., a clinically relevant target sequence, a target RNA sequence, and/or a target mRNA sequence. In some embodiments, machine learning models are used to model gRNA performances using primary gRNA sequences, and/or structural features for gRNA-target RNA scaffolds, as inputs, which results in high predictive accuracy for ADAR1, ADAR2, and/or APOBEC editing. In some embodiments, machine learning models are used to generate novel gRNA designs that overcome limitations described above. For instance, in some embodiments, input optimization is used to generate gRNA sequences with desired properties for facilitating nucleic acid editing, e.g., RNA editing.

In some embodiments, the machine learning methods, systems, and platforms described herein generate gRNA sequences that facilitate RNA editing in vivo. For example, in some embodiments, gRNAs sequences are generated that direct ADAR-mediated deamination of adenosine to inosine in target RNA. Inosine is then recognized by the translational machinery most frequently as guanine. In some embodiments, such targeted deamination is useful to correct G-A transitions found in genes linked to disorders, e.g., where the G-A transition results in expression of a protein with a point mutation or truncation contributing to the etiology of a disorder. In some embodiments, such targeted deamination is useful to introduce A-G transitions, e.g., to introduce a mutation in the amino acid sequence encoded by a target RNA or to introduce a stop codon causing a truncation of a protein. In some embodiments, such targeted deamination is useful to modify a splicing pattern of a gene transcript, e.g., where the A-G transition results in generation of a splice site (e.g., restoration of a wild type splice site or generation of a novel splice site), abrogation of an existing splice site (e.g., destruction of a mutant splice site or destruction of a wild type splice site), weakening of an existing splice site, or strengthening of an existing splice site. In some embodiments, such targeted deamination is useful to modify protein translation efficiency, e.g., by strengthening or weakening a translational initiation signal or by strengthening or weakening translational elongation. In some embodiments, such generative guide design is performed by models (e.g., generative adversarial networks (GANs), diffusion models, and/or denoising diffusion conditional GANs (ddGANs)) trained against one or more conditions (e.g., ADAR performance metrics).

Similarly, in some embodiments, the machine learning methods, systems, and platforms described herein generate gRNA sequences that facilitate RNA editing by directing APOBEC-mediated deamination of cytosine to uracil in target RNA. In some embodiments, such targeted deamination is useful to correct T→C transitions found in genes linked to disorders, e.g., where the T→C transition results in expression of a protein with a point mutation or truncation contributing to the etiology of a disorder. In some embodiments, such targeted deamination is useful to introduce C→U transitions, e.g., to introduce a mutation in the amino acid sequence encoded by a target RNA or to introduce a stop codon causing a truncation of a protein. In some embodiments, such targeted deamination is useful to modify a splicing pattern of a gene transcript, e.g., where the C→U transition results in generation of a splice site (e.g., restoration of a wild type splice site or generation of a novel splice site), abrogation of an existing splice site (e.g., destruction of a mutant splice site or destruction of a wild type splice site), weakening of an existing splice site, or strengthening of an existing splice site. In some embodiments, such targeted deamination is useful to modify protein translation efficiency, e.g., by strengthening or weakening a translational initiation signal or by strengthening or weakening translational elongation. In some embodiments, such generative guide design is performed by models (e.g., generative adversarial networks (GANs), diffusion models, and/or denoising diffusion conditional GANs (ddGANs)) trained against one or more conditions (e.g., ADAR performance metrics).

In some embodiments, the generated gRNA designs facilitate ADAR or APOBEC editing with high selectivity and specificity for any custom target. In some implementations, the gRNA designs obtained using the systems and methods disclosed herein outperform the gRNA from HTS used, in part, to train the models. Advantageously, in some embodiments, the novel gRNA designs exhibit primary, secondary, and/or tertiary sequence diversity beyond that of the original HTS screen. Moreover, in some implementations, these models are leveraged to improve and accelerate the gRNA discovery process by reducing the amount of running time and computational resources needed to interrogate the potential candidate gRNA space, and to expand the state of knowledge of the relationship between RNA primary sequence, secondary structure, tertiary structure, and ADAR or APOBEC activity.

In some aspects, the present disclosure harnesses the power of machine learning to evaluate, predict, determine, and/or generate polymer sequences (e.g., RNA, DNA, or protein sequences), for example for use in safe and efficient editing of a transcriptome of a subject (e.g., a human subject). In some implementations, the generated polymer sequences, such as guide RNAs and/or variant capsid proteins, can be or are used to treat, ameliorate, or fix genetic mutations in a subject. In some embodiments, a generated polymer sequence obtained as disclosed herein can be or is administered to a subject for use in gene therapy. For instance, as described above, delivery of DNA-encoded guide RNAs (gRNA) to recruit ADAR protein allows for programmable and precise RNA editing. ADAR is naturally a promiscuous enzyme with certain sequence editing preferences, but by screening millions of gRNAs, it is possible to learn the patterns and structures in RNA that specifically hone ADAR to edit a single site.

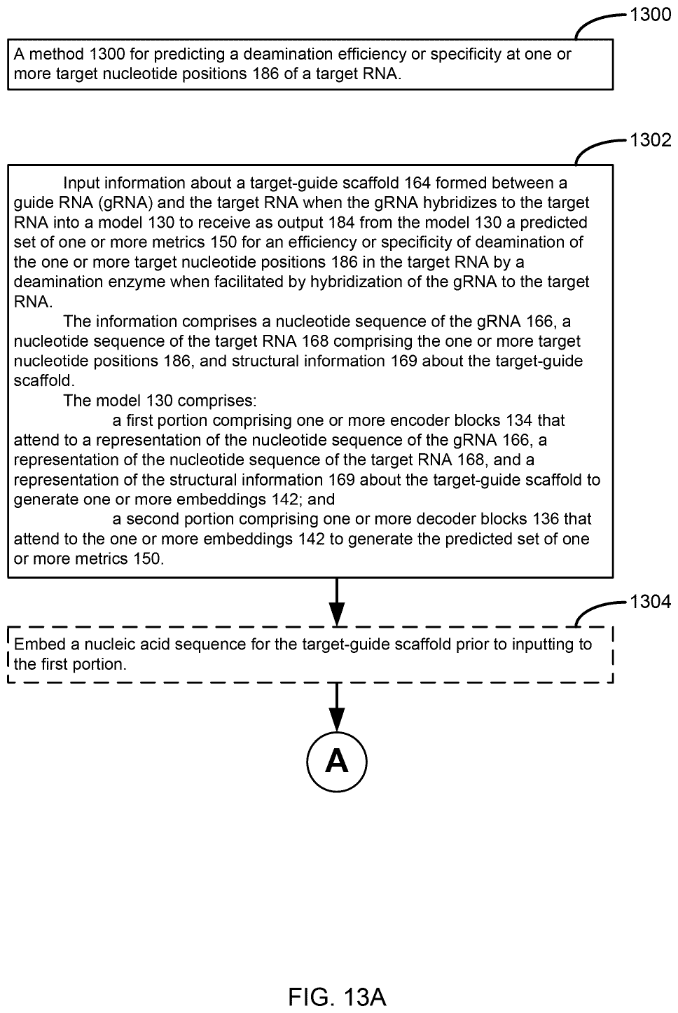

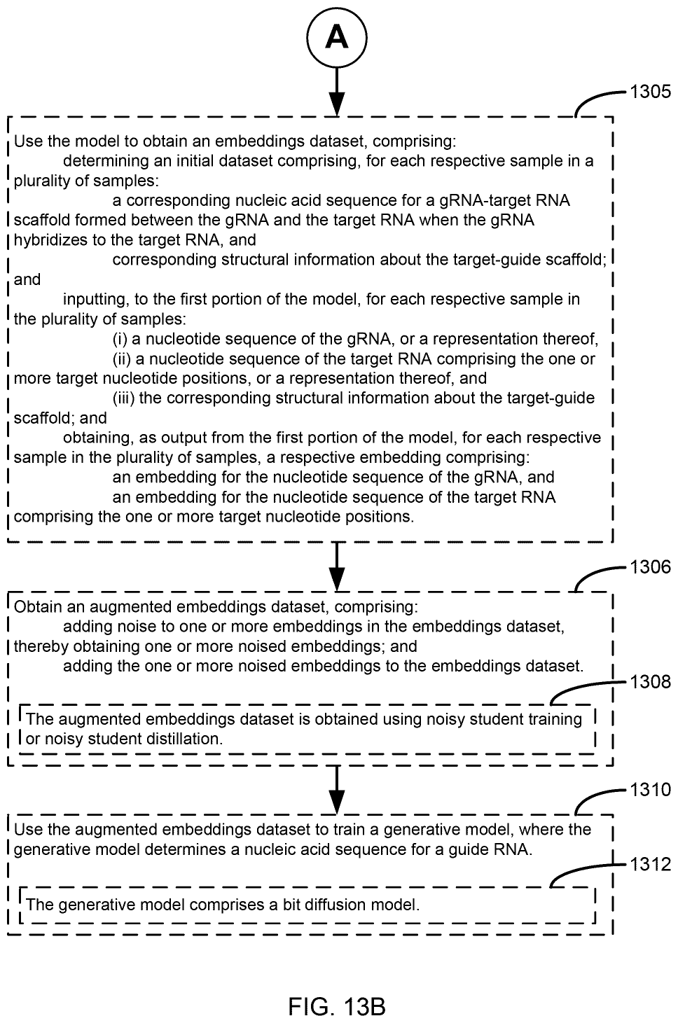

Referring to FIG. 2, one aspect of the present disclosure provides an example method 200 for optimizing a model to predict a deamination efficiency or specificity. Referring to FIGS. 5A-B and 6, another aspect of the present disclosure provides example methods 500 and 600 for predicting a deamination efficiency or specificity. Referring to FIGS. 13A-B, another aspect of the present disclosure provides example method 1300 for predicting a deamination efficiency or specificity at one or more target nucleotide positions of a target RNA.

In some embodiments, methods disclosed herein (e.g., method 200 and/or method 1300) are performed at a computer system comprising at least one processor and a memory storing at least one program for execution by the at least one processor. Referring to Block 202, in some embodiments, the method further includes obtaining a model 130 comprising a plurality of parameters 132 across a first block 134 and a second block 136. In some embodiments, each of the first block 134 and the second block 136 comprises an attention mechanism 138.

### Example Model Architecture.

In some embodiments, the model comprises a language model, a transformer model, a large language model (LLM), an encoder, a decoder, an encoder-decoder hybrid model, a generative pre-trained transformer (GPT) model, a Bidirectional Encoder Representations from Transformers (BERT) model, or a multiple sequence alignment (MSA) transformer model.

In some embodiments, the attention mechanism is selected from the group consisting of dot product attention, query-key-value attention, Luong attention, and Bahdanau attention.

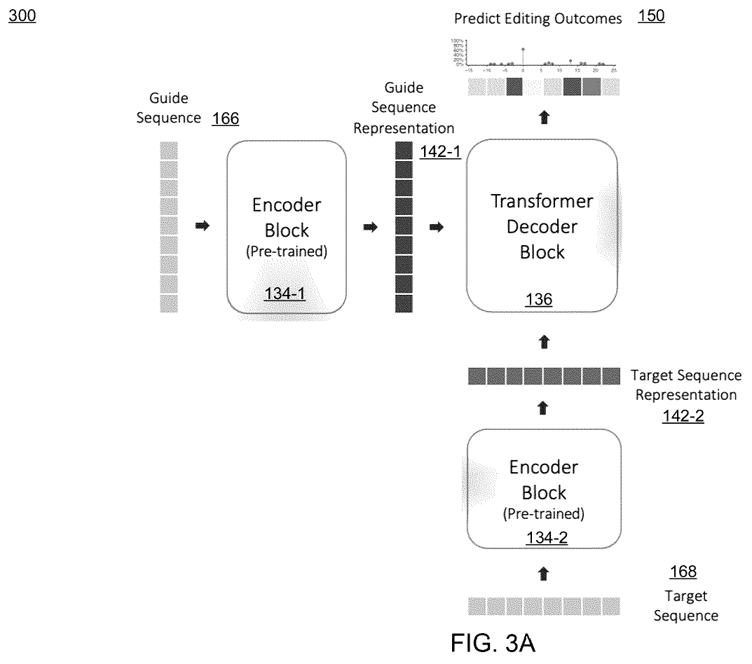

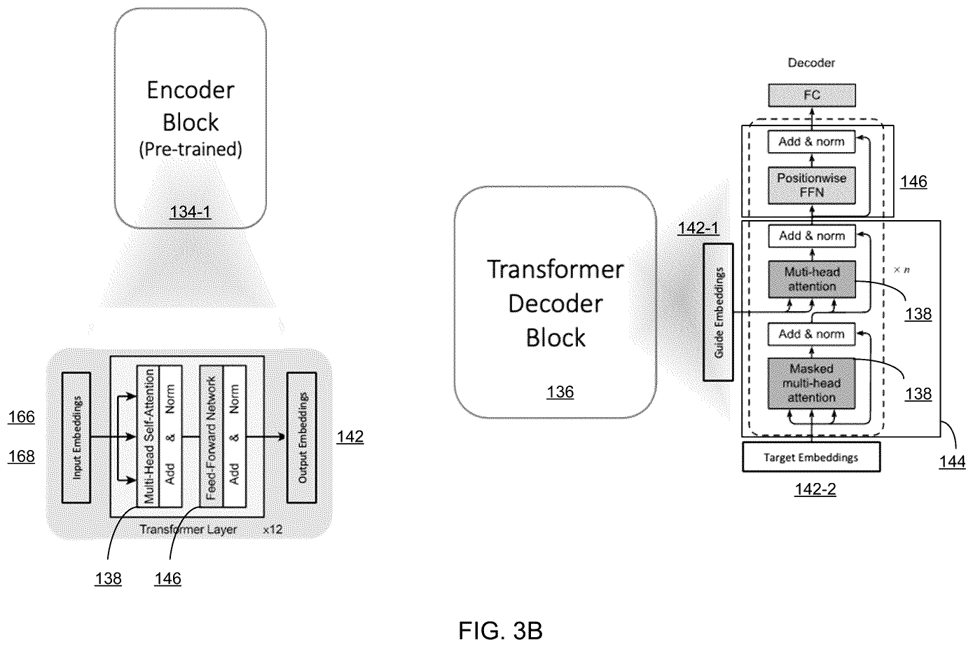

In some embodiments, referring, for example, to FIG. 3B, the attention mechanism 138 is applied directly to all or a portion of the data structure input into the model 166 and 168. In some embodiments, the attention mechanism is applied to an embedding of all or a portion of the data structure input into the model. In some embodiments, an attention mechanism is a mapping of a query (e.g., the data structure or embedding thereof) and a set of key-value pairs to an output where the query, keys, values, and output are all vectors. In some such embodiments, the output is computed as a weighted sum of the values, where the weight assigned to each value is computed by a compatibility function of the query with the corresponding key.

Example attention mechanisms are described in Chaudhari et al., Jul. 12, 2021 “An Attentive Survey of Attention Models,” arXiv:1904-02874v3, and Vaswani et al., “Attention is All You Need,” 31st Conference on Neural Information Processing Systems (NIPS 2017), Long Beach, California, USA, each of which is hereby incorporated by reference. The attention mechanism draws upon the inference that some portions of gRNA sequence, secondary structure, tertiary structure, or any combinations thereof, are more important than others and thus some portions (elements or sets of elements) within the data structure (or embedding thereof) are more important than other portions. The attention mechanism is trained to discover such importance using training gRNA and then apply this learned (trained) observation against the data structure (or embedding thereof) for the gRNA to form the attention embedding. Thus, the attention mechanism incorporates this notion of relevance by allowing the portion of the model downstream of the attention mechanism to dynamically pay attention to only certain parts of the input data, that help in performing the task at hand (e.g., predicting deamination efficiency of a gRNA) effectively.

In some embodiments, referring again to FIG. 3B, the model further comprises an output layer and each of the first block 134 and second block 136 comprise: an input layer, and a plurality of hidden layers comprising (i) a first portion that comprises the attention mechanism 138, and (ii) a second portion 146. In some embodiments, for each of the first block and the second block, the corresponding second portion comprises a neural network, a support vector machine, a Naive Bayes model, a nearest neighbor model, a boosted trees model, a random forest model, a decision tree, or a clustering model. In some embodiments, one or both of the first portion and the second portion further comprise one or more addition and normalization layers and/or one or more feed-forward layers. In some embodiments, an addition and normalization layer sums and/or normalizes one or more outputs of one or more previous layers. In some embodiments, a feed-forward layer performs a scalar transformation on one or more outputs of one or more previous layers. In some embodiments, the model further comprises one or more fully connected layers. In some embodiments, a fully connected layer comprises a neural network in which each neuron applies a linear transformation to the input vector through a weights matrix. As a result, all possible connections layer-to-layer are present, meaning every input of the input vector influences every output of the output vector.

In some embodiments, the second portion of the model comprises an extreme gradient boost (XGBoost) model. In some embodiments, the second portion of the model comprises a convolutional or graph-based neural network.

In some embodiments, the first block is an encoder block and the second block is a decoder block. In some embodiments, the model comprises one or more encoder blocks and one or more decoder blocks. In some embodiments, the model comprises two or more encoder blocks and a decoder block. In some embodiments, the model consists of two encoder blocks and a decoder block.

In some embodiments, the model comprises at least 1, at least 2, at least 3, at least 4, at least 5, at least 6, or at least 7 encoder blocks. In some embodiments the model comprises no more than 10, no more than 7, no more than 5, no more than 3, or no more than 2 encoder blocks. In some embodiments, the model consists of from 1 to 5, from 2 to 8, or from 5 to 10 encoder blocks. In some embodiments, the model includes another range of encoder blocks starting no lower than 1 encoder block and ending no higher than 10 encoder blocks. In some embodiments, the model comprises at least 1, at least 2, at least 3, or at least 4 decoder blocks. In some embodiments the model comprises no more than 5, no more than 3, or no more than 2 decoder blocks. In some embodiments, the model consists of from 1 to 3, from 2 to 4, or from 2 to 5 decoder blocks. In some embodiments, the model includes another range of decoder blocks starting no lower than 1 decoder block and ending no higher than 5 decoder blocks.

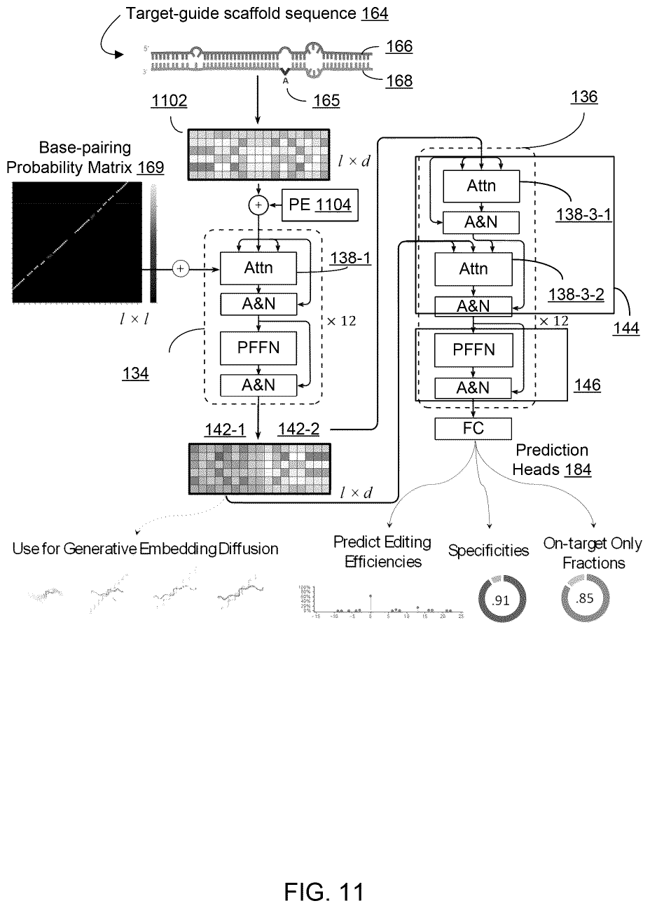

In some embodiments, the model consists of a single encoder block and a single decoder block. For instance, another example model architecture is illustrated in FIG. 11 and disclosed in further detail below.

Advantageously, transformer-based models can handle inputs with variable lengths, making such models generalizable to different micro-footprints or macro-footprints for in-cell experiments. Additionally, transformers are less prone to overfitting, allowing for easy integration of historical or future datasets to further enhance the model performance.

### Encoder Blocks.

In some embodiments, the encoder block receives, as input, for each sample in a plurality of samples (e.g., test samples and/or training samples), one or more of (i) a first portion of a nucleic acid sequence for a gRNA-target RNA scaffold that corresponds to a nucleic acid sequence for the gRNA, or a representation thereof, (ii) a second portion of the nucleic acid sequence for a gRNA-target RNA scaffold that corresponds to the nucleic acid sequence for the target RNA, or a representation thereof, and (iii) structural information for the nucleic acid sequence comprising a plurality of structural features formed between the gRNA and the target RNA when the gRNA hybridizes to the target RNA.

In some embodiments, the encoder block receives, as input, for each sample in the plurality of samples (e.g., test samples and/or training samples), one or more of the first portion of a nucleic acid sequence for a gRNA-target RNA scaffold that corresponds to a nucleic acid sequence for the gRNA, or the representation thereof, and the structural information for the nucleic acid sequence comprising a plurality of structural features formed between the gRNA and the target RNA when the gRNA hybridizes to the target RNA. In some embodiments, the decoder block receives, as input, at least an output from the encoder block responsive to inputting to the encoder block the first portion of a nucleic acid sequence for a gRNA-target RNA scaffold that corresponds to a nucleic acid sequence for the gRNA, or the representation thereof, and the structural information for the nucleic acid sequence comprising a plurality of structural features formed between the gRNA and the target RNA when the gRNA hybridizes to the target RNA. In some embodiments, the decoder block further receives, as input, one or more of the second portion of the nucleic acid sequence for a gRNA-target RNA scaffold that corresponds to the nucleic acid sequence for the target RNA, or a representation thereof, and the structural information for the nucleic acid sequence comprising a plurality of structural features formed between the gRNA and the target RNA when the gRNA hybridizes to the target RNA.

In some embodiments, the encoder block receives, as input, for each sample in the plurality of samples (e.g., test samples and/or training samples), (i) the first portion of a nucleic acid sequence for a gRNA-target RNA scaffold that corresponds to a nucleic acid sequence for the gRNA, or a representation thereof, (ii) the second portion of the nucleic acid sequence for a gRNA-target RNA scaffold that corresponds to the nucleic acid sequence for the target RNA, or a representation thereof, and (iii) the structural information for the nucleic acid sequence comprising a plurality of structural features formed between the gRNA and the target RNA when the gRNA hybridizes to the target RNA.

In some embodiments, the plurality of samples (e.g., test samples and/or training samples) comprises at least 1000 samples, at least 10,000 samples, at least 100,000 samples, at least 500,000 samples, at least 1×10⁶ samples, at least 1×10⁷ samples, or at least 1×10⁸ samples. In some embodiments, the plurality of samples comprises no more than 1×10⁹, no more than 1×10⁸, no more than 1×10⁷, no more than 1×10⁶, no more than 500,000, no more than 100,000, or no more than 10,000 samples. In some embodiments, the plurality of samples consists of from 1,000 to 50,000, from 10,000 to 500,000, from 100,000 to 1×10⁷, or from 1×10⁶ to 1×10⁹ samples. In some embodiments, the plurality of samples falls within another range starting no lower than 1,000 samples and ending no higher than 1×10⁹ samples.

In some embodiments, the plurality of samples comprises a plurality of test samples. In some embodiments, the plurality of test samples is inputted into a trained, retrained, or pretrained model. In some embodiments, the plurality of samples comprises a plurality of training samples. In some embodiments, the plurality of training samples is inputted into a trained, retrained, or pretrained model.

In some embodiments, the structural information comprises any of the structural information disclosed herein (see, for instance, the section entitled “Retraining models,” below). In some embodiments, the structural information comprises secondary structural features, tertiary structural features, or a combination thereof.

In some embodiments, the structural information comprises, for each respective position in a plurality of positions in the corresponding nucleic acid sequence for a gRNA-target RNA scaffold, a respective probability that a nucleotide base at the respective position will form a base-pair interaction with a nucleotide base at every other position in the plurality of positions other than the respective position. In some embodiments, the structural information comprises a base-pairing probability matrix. In some embodiments, a base-pairing probability matrix is provided as a vector, or as one or more vectors. For example, in some embodiments, a base-pairing probability matrix is provided as separate inputs (e.g., vectors), for each of the target RNA sequence and the gRNA sequence. In some embodiments, a base-pairing probability matrix comprises dimensions of l×l, where l is a length of the number of positions in the plurality of positions in the nucleic acid sequence for the gRNA-target RNA scaffold, and where each value or indication in the matrix is the respective probability that a nucleotide base at the respective position will form a base-pair interaction with a nucleotide base at every other position in the plurality of positions other than the respective position.

In some embodiments, the input information further comprises one or more conditions (e.g., conditions 410), such as a set of one or more target metrics. In some embodiments, a respective target metric in the set of one or more target metrics comprises a metric for an efficiency or specificity of deamination of a target nucleotide position in a target RNA by a deamination enzyme (e.g., an ADAR protein) when facilitated by hybridization of a gRNA to the target RNA. Metrics contemplated for use as target metrics are described in further detail elsewhere herein (see, e.g., the section entitled “Retraining models,” below).

### Input Representations.

In some embodiments, systems and methods disclosed herein (e.g., method 200) further include obtaining a representation of all or a portion of a sample in the plurality of samples, prior to inputting into the model (e.g., at an encoder block and/or a decoder block). In some embodiments, a method disclosed herein includes obtaining a representation of one or more of (i) the first portion of a nucleic acid sequence for a gRNA-target RNA scaffold that corresponds to a nucleic acid sequence for the gRNA, (ii) the second portion of the nucleic acid sequence for a gRNA-target RNA scaffold that corresponds to the nucleic acid sequence for the target RNA, and (iii) the structural information for the nucleic acid sequence that comprises a plurality of structural features formed between the gRNA and the target RNA when the gRNA hybridizes to the target RNA, prior to inputting into the model (e.g., at an encoder block and/or a decoder block).

In some embodiments, systems and methods disclosed herein (e.g., method 200) include obtaining a representation of the first portion of a nucleic acid sequence for a gRNA-target RNA scaffold that corresponds to a nucleic acid sequence for the gRNA, and/or the second portion of the nucleic acid sequence for the gRNA-target RNA scaffold that corresponds to the nucleic acid sequence for the target RNA, prior to inputting into the encoder block of the model.

In some embodiments, systems and methods disclosed herein include obtaining a representation of the structural information for the nucleic acid sequence, prior to inputting into the encoder block of the model.

In some embodiments, the representation comprises an embedding.

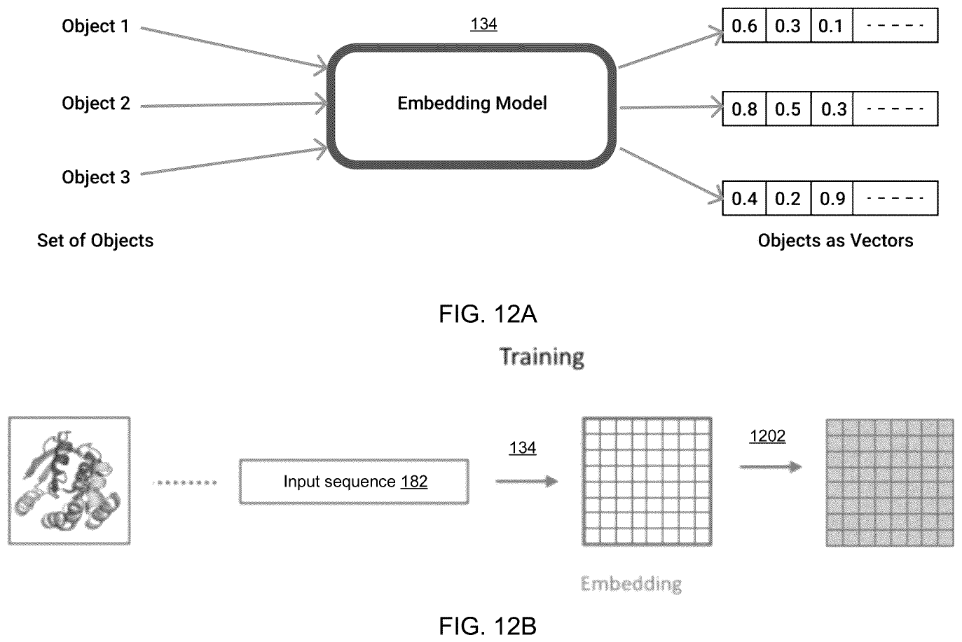

Generally, embeddings comprise a representation (e.g., in tensor form) of an object, such as a nucleic acid or amino acid sequence, a condition, and/or a representation thereof. An embedding can refer to a high dimensional representation in a continuous space of any data that is not canonically represented in this way. For example, RNA sequences can be represented with strings or one-hot-encoding. However, in some implementations, such representations do not capture the full complexity or structure of the RNA sequence. In some embodiments, as illustrated in FIG. 12A, an embedded object, such as a nucleotide sequence, is represented as a tensor or vector of values.

In some embodiments, data in an embedding space is represented in a continuous fashion, where similar sequences are represented by similar embeddings (e.g., tensors or vectors). In some embodiments, embeddings are also contextual; thus allowing greater complexity to be captured. Consider, for example, each nucleotide A of a sequence having the same one hot encoding, in contrast to embeddings in which contextual information is represented. Advantageously, in some embodiments, embeddings allow machine learning models to capture and learn complex data, including contextual information.

In some implementations, embeddings capture semantic relationships or context between elements of the representation (e.g., positions in a sequence). This is useful, for instance, for preserving position-specific information and/or inter-position interactions and dependencies in polymer sequences. Methods, models, and algorithms for embedding suitable for use in the present disclosure are known in the art, including but not limited to such examples as principal component analysis (PCA), singular value decomposition (SVD), Word2Vec, Sequence2Vec, Gene2Vec, kmer2vec, seq2seq, and/or BERT. See, for example, Mokhtarani, “Embeddings in Machine Learning: Everything You Need to Know,” 2021, available on the Internet at featureform.com/post/the-definitive-guide-to-embeddings.

In some embodiments, the embedding for a target sequence comprises a gRNA sequence for the target sequence. In some embodiments, the embedding for a target sequence comprises a complementary sequence for the target sequence. In some embodiments, the complementary sequence consists of an exact complement of the target sequence.

In some embodiments, an embedding comprises dimensions of l×d, where l is the length of the number of positions in a plurality of positions in the target-guide scaffold, and where d is a number of components, or dimensions, representing the complexity of the data captured in the embedding. For instance, FIG. 11 illustrates inputting an embedding 1102 of a target-guide scaffold sequence into the encoder block 134 of the model.

Alternately or additionally, in some embodiments, the representation comprises an encoding. In some embodiments, the encoding is a 5-bit encoding. In some embodiments, the encoding is a 6-bit encoding, 7-bit encoding, 8-bit encoding, or higher bit encoding. For example, 5-bit encoding allows for binary encoding with 32 different characters, {0,0,0,0,0} through {1,1,1,1,1}. As such, each natural residue in a sequence (e.g., amino acid, nucleic acid, etc.) can be encoded with a different character. In some embodiments, residues in a sequence are assigned to different characters without any particular considerations, e.g., randomly assigned or assigned alphabetically. In some embodiments, one or more characteristics are considered when assigning residues to characters. For example, in some embodiments, residues in a sequence having similar biophysical properties are assigned to characters sharing positional values. In some embodiments, the encoding comprises positional encoding. For instance, FIG. 11 illustrates inputting a positional encoding 1104 of a target-guide scaffold sequence and a positional encoding of structural information 169 into the encoder block 134 of the model. In some embodiments, the encoding comprises one hot encoding.

In some embodiments, the encoding comprises a one hot encoding of each of a plurality of possible residue identities (e.g., nucleic acid and/or amino acid residues) such that the value indicates a presence or absence of the respective residue identity at the respective position. Consider, for example, a plurality of possible amino acid identities comprising at least 20 amino acid identities or at least 22 amino acid identities. In an example embodiment, for a first position in the amino acid sequence, the corresponding vector is [1,0,0,0,0,0,0,0,0,0,0,0,0,0,0,0,0,0,0,0], where 1 indicates a presence for a first amino acid identity and 0 indicates an absence of all other amino acid identities. In some embodiments, the encoding comprises a one hot encoding of each of one or more feature properties such that the value indicates that a residue comprises or does not comprise the respective feature property.

### Encoder Attention.

In some embodiments, a respective encoder block (e.g., in one or more encoder blocks) comprises one or more attention mechanisms. In some embodiments, a respective encoder block comprises at least 1, at least 2, at least 3, or at least 4 attention mechanisms. In some embodiments, a respective encoder block comprises no more than 5, no more than 4, no more than 3, or no more than 2 attention mechanisms. In some embodiments, a respective encoder block consists of from 1 to 3, from 2 to 4, or from 2 to 5 attention mechanisms. In some embodiments, a respective encoder block includes another range of attention mechanisms starting no lower than 1 attention mechanism and ending no higher than 5 attention mechanisms.

In some embodiments, the encoder block includes an attention mechanism that attends to one or more of: a representation of the nucleotide sequence of the gRNA, a representation of the nucleotide sequence of the target RNA, and a representation of the structural information. In some embodiments, the encoder block includes an attention mechanism that attends to each of the representation of the nucleotide sequence of the gRNA, the representation of the nucleotide sequence of the target RNA, and the representation of the structural information.

In some embodiments, an encoder block (e.g., a first block) generates, as output, a representation of an input (e.g., an embedding).

### Encoder Output.

In some embodiments, the encoder block receives, as input, one or more of (i) a first portion of a nucleic acid sequence for a gRNA-target RNA scaffold that corresponds to a nucleic acid sequence for the gRNA, or a representation thereof, (ii) a second portion of the nucleic acid sequence for the gRNA-target RNA scaffold that corresponds to the nucleic acid sequence for the target RNA, or a representation thereof, and (iii) structural information for the nucleic acid sequence that comprises a plurality of structural features formed between the gRNA and the target RNA when the gRNA hybridizes to the target RNA. In some such embodiments, the encoder block generates, as output, a corresponding representation of one or more of (i) the first portion of a nucleic acid sequence for the gRNA-target RNA scaffold that corresponds to the nucleic acid sequence for the gRNA, (ii) the second portion of the nucleic acid sequence for the gRNA-target RNA scaffold that corresponds to the nucleic acid sequence for the target RNA, and (iii) the structural information for the nucleic acid sequence.

In some embodiments, the encoder block receives, as input, (i) a first portion of a nucleic acid sequence for a gRNA-target RNA scaffold that corresponds to a nucleic acid sequence for the gRNA, or a representation thereof, (ii) a second portion of the nucleic acid sequence for the gRNA-target RNA scaffold that corresponds to the nucleic acid sequence for the target RNA, or a representation thereof, and (iii) structural information for the nucleic acid sequence that comprises a plurality of structural features formed between the gRNA and the target RNA when the gRNA hybridizes to the target RNA, and the encoder block generates, as output, a corresponding representation of the first portion and the second portion of the nucleic acid sequence for the gRNA-target RNA scaffold.

In some embodiments, the representation generated by the encoder is an embedding. In some embodiments, the representation generated by the encoder is an embedding of (i) a first portion of a nucleic acid sequence for a gRNA-target RNA scaffold that corresponds to a nucleic acid sequence for the gRNA and/or (ii) a second portion of the nucleic acid sequence for the gRNA-target RNA scaffold that corresponds to the nucleic acid sequence for the target RNA. In some embodiments, the embedding is any of the embodiments disclosed elsewhere herein, for example, in the section entitled “Input representations,” above. For instance, as illustrated in FIGS. 11 and 12A-B, in some implementations, an embedding model (e.g., encoder block 134) accepts an input object (e.g., input sequence 182 and/or target-guide scaffold sequence 164) and generates an embedding (e.g., embeddings 142).

In some embodiments, the encoder block considers (e.g., accepts as input, processes, and/or attends to) information obtained from various inputs to generate one or more embeddings. In some embodiments, the encoder block considers all or a portion of the input to the model to generate the one or more embeddings. In some embodiments, the encoder block considers all or a portion of the input to the model, or representations or derivatives thereof (e.g., a sequence having complementarity to an input sequence), to generate the one or more embeddings. In some embodiments, where the encoder block generates a plurality of embeddings, the encoder block considers the same or different information to generate a first embedding relative to the information considered in order to generate a second embedding.

For instance, in some embodiments, the encoder block generates, as output, (i) a first embedding for a nucleotide sequence of the gRNA, and/or (ii) a second embedding for a nucleotide sequence of the target RNA comprising one or more target nucleotide positions. In some embodiments, the encoder block generates the first embedding for the nucleotide sequence of the gRNA by considering (e.g., accepting as input, processing, and/or attending to) one or more of: (i) the nucleotide sequence of the gRNA, (ii) the nucleotide sequence of the target RNA, and (iii) the structural information about the target-guide scaffold. In some embodiments, the encoder block generates the first embedding for the nucleotide sequence of the gRNA by considering only the nucleotide sequence of the target RNA. In some embodiments, the encoder block generates the first embedding for the nucleotide sequence of the gRNA by considering the nucleotide sequence of the target RNA and a nucleotide sequence having perfect complementarity to the nucleotide sequence of the target RNA. In some embodiments, the encoder block generates the first embedding for the nucleotide sequence of the gRNA by considering the nucleotide sequence of the target RNA, a nucleotide sequence having perfect complementarity to the nucleotide sequence of the target RNA, and structural information about a target-sequence scaffold formed between the target RNA and the nucleotide sequence having perfect complementarity to the nucleotide sequence of the target RNA. In some embodiments, the encoder block generates the second embedding for the nucleotide sequence of the target RNA by considering one or more of: (i) the nucleotide sequence of the gRNA, (ii) the nucleotide sequence of the target RNA, and (iii) the structural information about the target-guide scaffold. In some embodiments, the encoder block generates the second embedding for the nucleotide sequence of the target RNA by considering: only the nucleotide sequence of the target RNA; the nucleotide sequence of the target RNA and a nucleotide sequence having perfect complementarity to the nucleotide sequence of the target RNA; or the nucleotide sequence of the target RNA, the nucleotide sequence having perfect complementarity to the nucleotide sequence of the target RNA, and structural information about a target-sequence scaffold formed between the target RNA and the nucleotide sequence having perfect complementarity to the nucleotide sequence of the target RNA. In some embodiments, the encoder block considers (e.g., accepts as input, processes, and/or attends to) the same or different information to generate the first embedding and the second embedding.

In some embodiments, the one or more embeddings comprises an embedding for a nucleic acid sequence for a target-guide scaffold, where the nucleic acid sequence of the target-guide scaffold comprises a nucleotide sequence of the gRNA and a nucleotide sequence of the target RNA comprising the one or more target nucleotide positions. In some embodiments, the encoder block generates the embedding for the nucleic acid sequence for the target-guide scaffold by considering one or more of: (i) the nucleotide sequence of the gRNA, (ii) the nucleotide sequence of the target RNA, and (iii) the structural information about the target-guide scaffold. In some embodiments, the encoder block generates the embedding for the nucleic acid sequence for the target-guide scaffold by considering: only the nucleotide sequence of the target RNA; the nucleotide sequence of the target RNA and a nucleotide sequence having perfect complementarity to the nucleotide sequence of the target RNA; or the nucleotide sequence of the target RNA, the nucleotide sequence having perfect complementarity to the nucleotide sequence of the target RNA, and structural information about a target-sequence scaffold formed between the target RNA and the nucleotide sequence having perfect complementarity to the nucleotide sequence of the target RNA.

In some embodiments, the representation outputted from the encoder is used to generate datasets comprising nucleic acid sequence representations, optionally by adding noise 1202, as discussed in further detail below.

In some embodiments, the model is a predictive model that generates, as output, responsive to receiving input, a predicted set of one or more metrics for the efficiency or specificity of deamination of one or more target nucleotide positions in the target RNA by a deamination enzyme when facilitated by hybridization of the gRNA to the target RNA.

### Decoder Blocks.

In some embodiments, the model comprises one or more decoder blocks that receives, as input, an output from one or more encoder blocks. In some embodiments, the model comprises a decoder block that receives, as input, an output from an encoder block, where the output from the encoder block is an embedding of (i) a first portion of a nucleic acid sequence for a gRNA-target RNA scaffold that corresponds to a nucleic acid sequence for the gRNA and/or (ii) a second portion of the nucleic acid sequence for the gRNA-target RNA scaffold that corresponds to the nucleic acid sequence for the target RNA.

In some embodiments, a respective decoder block (e.g., in one or more decoder blocks) comprises one or more attention mechanisms. In some embodiments, a respective decoder block comprises at least 1, at least 2, at least 3, or at least 4 attention mechanisms. In some embodiments, a respective decoder block comprises no more than 5, no more than 4, no more than 3, or no more than 2 attention mechanisms. In some embodiments, a respective decoder block consists of from 1 to 3, from 2 to 4, or from 2 to 5 attention mechanisms. In some embodiments, a respective decoder block includes another range of attention mechanisms starting no lower than 1 attention mechanism and ending no higher than 5 attention mechanisms. In some embodiments, the decoder block accepts, as input, one or more outputs generated by the encoder block. In some embodiments, the decoder block comprises one or more attention mechanisms, where each respective attention mechanism attends to one or more respective outputs generated by the encoder block.

In some embodiments, a respective decoder block comprises a first attention mechanism and a second attention mechanism. In some embodiments, a respective decoder block comprises a corresponding a first portion and a second portion, where the first portion comprises a first attention mechanism that receives, as input, an embedding of a nucleotide sequence of a gRNA, and a second attention mechanism that receives, as input, an embedding of a nucleotide sequence for a target RNA comprising one or more target nucleotide positions. In some embodiments, a respective decoder block further comprises a third attention mechanism that attends to structural information for the nucleic acid sequence that comprises a plurality of structural features formed between the gRNA and the target RNA when the gRNA hybridizes to the target RNA, or a representation thereof.

### Decoder Output.

In some embodiments, the decoder block generates, as output, responsive to receiving input, a predicted set of one or more metrics, or a representation thereof, for the efficiency or specificity of deamination of one or more target nucleotide positions in the target RNA by a deamination enzyme when facilitated by hybridization of the gRNA to the target RNA. In some embodiments, the deamination enzyme is an Adenosine Deaminase Acting on RNA (ADAR) protein.

In some embodiments, an output from the model comprises an output from the decoder block. In some embodiments, the model further comprises an output layer that receives, as input, an output from the decoder block and generates, as output, a predicted set of one or more metrics for the efficiency or specificity of deamination of one or more target nucleotide positions in the target RNA by a deamination enzyme (e.g., an ADAR protein) when facilitated by hybridization of the gRNA to the target RNA.

In some embodiments, the output from the model comprises any of the outputs disclosed elsewhere herein, including, but not limited to, any of the embodiments for one or more metrics for an efficiency or specificity of deamination of a target nucleotide position in a target RNA by a deamination enzyme (e.g., an ADAR protein) when facilitated by hybridization of a gRNA to the target RNA (see, e.g., the section entitled “Retraining models,” below).

### Embedding Datasets.

In some embodiments, as indicated above, the representation outputted from the encoder is used to generate datasets comprising nucleic acid sequence representations. In some embodiments, the presently disclosed systems and methods comprise obtaining, as output from the encoder, a corresponding representation for each sample in a plurality of samples (e.g., test samples). For instance, in some embodiments, the presently disclosed systems and methods comprise obtaining, as output from the encoder, a corresponding representation for each gRNA-target RNA scaffold, formed between the gRNA and the target RNA when the gRNA hybridizes to the target RNA, in a plurality of gRNA-target RNA scaffolds. In some embodiments, the presently disclosed systems and methods comprise obtaining, as output from the encoder, a corresponding representation for each target RNA in a plurality of target RNAs. In some embodiments, each corresponding representation comprises an embedding of a nucleotide sequence for the target RNA and/or an embedding of a nucleotide sequence for a gRNA corresponding to the target RNA (e.g., in a gRNA-target RNA scaffold).

In some embodiments, the presently disclosed systems and methods further include obtaining an embedding dataset comprising a plurality of embeddings, where each respective embedding in the plurality of embeddings comprises an embedding of a nucleotide sequence for the target RNA and/or an embedding of a nucleotide sequence for a gRNA corresponding to the target RNA.

Advantageously, in some embodiments, the embeddings generated by the encoder blocks further include meaningful information on RNA biology, enabling other tasks through transfer learning.

In some embodiments, referring again to FIGS. 11 and 12B, the embedding datasets are further modified or augmented, optionally by adding noise 1202. In some embodiments, the method further includes adding noise to each respective embedding in the plurality of embeddings in the embedding dataset, thereby generating a noised embedding dataset. For instance, in some embodiments, the method further includes adding noise to the embedded nucleic acid sequence for each respective target in the plurality of targets, such that each respective embedding in the plurality of embeddings is a noised embedding. In some embodiments the noise comprises one or more of dropout, stochastic depth, and data augmentation.

Alternatively or additionally, in some embodiments, the presently disclosed systems and methods further include performing an augmentation of the embedding dataset, prior to training the model. In some embodiments, the augmentation comprises Noisy Student Training, where the embedding dataset is augmented by adding one or more noised embeddings. For instance, in some embodiments, the augmentation comprises: adding noise to one or more embeddings in the embeddings dataset, thereby obtaining one or more noised embeddings; and adding the one or more noised embeddings to the embeddings dataset.

The Noisy Student algorithm is a semi-supervised learning technique designed to improve model performance by combining supervised and semi-supervised learning. First, a “teacher” model is trained on a labeled dataset, which then generates noisy augmented versions of the data, including both the original labeled and additional unlabeled data. A “student” model is then trained using this augmented dataset, thereby incorporating both the noisy augmented labels and the predictions from the teacher model. Without being limited to any one theory of operation, this process enhances generalization and robustness of the student model by effectively utilizing the additional unlabeled data. Employing noise in the student model's training aids in regularizing the student model and increasing resilience to overfitting. Noisy Student training is further described, for example, in Xie et al., “Self-training with Noisy Student improves ImageNet classification,” arXiv 2020, doi: 10.48550/arXiv.1911.04252: 1911.04252, which is hereby incorporated herein by reference in its entirety.

In some embodiments, the one or more noised embeddings are obtained by adding noise to an embedding in the embedding dataset. In some embodiments the noise comprises one or more of dropout (e.g., dropping units (or connections) in a model, such as a neural network, during training), stochastic depth (e.g., introducing variability into a training process by dropping layers of a model at varying training iterations), and data augmentation (e.g., applying transformations to input data, such as rotations, translations, or scaling). In some embodiments, the noise comprises label noise (e.g., altering labels of training data), input noise (e.g., adding perturbations to input data, such as Gaussian noise), or label smoothing (e.g., modifying sample labels, for instance, by blending them with a uniform distribution). Suitable embodiments for noise contemplated for use in the present disclosure are further described, for example, in Xie et al., “Self-training with Noisy Student improves ImageNet classification,” arXiv 2020, doi: 10.48550/arXiv.1911.04252: 1911.04252, which is hereby incorporated herein by reference in its entirety.

In some embodiments, the presently disclosed systems and methods further comprise using the embedding dataset to train a model, such as a generative model (e.g., to predict gRNA sequences). For instance, as illustrated in Examples 5-7 below, bit diffusion models trained on noisy student-augmented training datasets generated gRNA designs for target RNAs that exhibited higher specificity compared to gRNA designs generated by bit diffusion models trained on non-augmented training datasets.

FIG. 11 illustrates an example schematic for a transformer-based model comprising an encoder-decoder architecture, in accordance with some embodiments of the present disclosure.

Referring to FIG. 11, in some embodiments, methods disclosed herein provide inputting to a model, information about a target-guide scaffold 164 formed between a guide RNA (gRNA) and the target RNA when the gRNA hybridizes to the target RNA, where the information comprises a nucleotide sequence of the gRNA 166, a nucleotide sequence of the target RNA 168 comprising the one or more target nucleotide positions, and structural information 169 about the target-guide scaffold. In some embodiments, the model comprises a first portion comprising an encoder block 134 and a second portion comprising a decoder block 136. In some embodiments, the first portion comprising the encoder block 134 comprises one or more encoders (e.g., 12) each comprising an attention mechanism 138-1 (e.g., multi-head attention) that attends to a representation (e.g., an embedding 1102) of the nucleotide sequence of the gRNA 166, a representation (e.g., an embedding 1102) of the nucleotide sequence of the target RNA 168, and the structural information 169 about the target-guide scaffold to generate one or more embeddings 142. In some embodiments, each of the embedding 1102 for the nucleotide sequence for the target-guide scaffold and the structural information 169 about the target-guide scaffold are further encoded using positional encoding (“PE”) 1104, prior to inputting into the encoder block 134. In some embodiments, the encoder block 134 generates, as output, responsive to the inputting, an embedding of (i) the nucleotide sequence of the gRNA 142-1 and (ii) the nucleotide sequence for the target RNA 142-2 comprising the one or more target nucleotide positions. In some embodiments, the encoder further comprises one or more input layers, one or more addition and normalization layers (“A&N”), and/or one or more feed-forward network layers (e.g., position-wise feed-forward networks (“PFFN”)).

FIG. 11 further illustrates, in some embodiments, where the second portion comprising the decoder block 136 comprises a corresponding a first sub-portion 144 and a second sub-portion 146, where the first sub-portion 144 comprises a first attention mechanism 138-3-1 that receives, as input 148-1, the embedding of the nucleotide sequence of the gRNA 142-1, and a second attention mechanism 138-3-2 that receives, as input, the embedding of the nucleotide sequence for the target RNA 142-2 comprising the one or more target nucleotide positions. In some embodiments, the decoder block 136 generates, as output 150, a predicted set of one or more metrics for the efficiency or specificity of deamination of the target nucleotide position in the target RNA by the deamination enzyme (e.g., the ADAR protein) when facilitated by hybridization of the test gRNA to the target RNA, or a representation thereof. In some embodiments, the output 150 from the decoder is further used to generate output 184 from the model, for instance, via a fully connected layer (“FC”), where the output 184 from the model comprises a predicted set of one or more metrics for the efficiency or specificity of deamination of the target nucleotide position in the target RNA by the deamination enzyme (e.g., the ADAR protein) when facilitated by hybridization of the test gRNA to the target RNA. In some embodiments, the output from the model comprises any of the outputs disclosed elsewhere herein, including, but not limited to, any of the embodiments for one or more metrics for an efficiency or specificity of deamination of a target nucleotide position in a target RNA by a deamination enzyme (e.g., an ADAR protein) when facilitated by hybridization of a gRNA to the target RNA (see, e.g., the section entitled “Retraining models,” below).

Referring again to FIG. 11, in some embodiments, methods disclosed herein comprise inputting information to a model for each respective target RNA in a plurality of target RNAs (e.g., test samples). In some embodiments, the encoder block 134 generates, as output, for each respective target RNA in the plurality of target RNAs, responsive to the inputting, an embedding of (i) the nucleotide sequence of the gRNA 142-1 and (ii) the nucleotide sequence for the target RNA 142-2 comprising the one or more target nucleotide positions, thereby obtaining a plurality of embeddings. In some embodiments, methods disclosed herein further comprise obtaining an embedding dataset comprising the plurality of embeddings. In some embodiments, methods disclosed herein further include using 1106 the embedding dataset to train a model, such as a generative model (e.g., to generate gRNA sequences or representations thereof 120).

In some embodiments, referring, for example, to FIGS. 3A-B, the first block is an encoder block 134, the second block is a decoder block 136, and the model receives, responsive to inputting information comprising a corresponding nucleic acid sequence for a gRNA-target RNA scaffold: at the input layer of the first block, a portion of the nucleic acid sequence for the gRNA-target RNA scaffold that corresponds to the nucleic acid sequence for the gRNA 166, or a representation thereof, and, at the input layer of the second block, a portion of the nucleic acid sequence for the gRNA-target RNA scaffold that corresponds to the nucleic acid sequence for the target RNA 168, or a representation thereof 142-2.

In some embodiments, referring again to FIGS. 3A-B, the model 130 comprises a first encoder block 134-1, a second encoder block 134-2, and a decoder block 136, where the first encoder block 134-1 comprises a first set of parameters 132-1, in a plurality of parameters of the model, the second encoder block 134-2 comprises a second set of parameters 132-2, in the plurality of parameters of the model, and the decoder block 136 comprises a third set of parameters 132-3, in the plurality of parameters of the model.

In some embodiments, responsive to receiving, as input to the model, information comprising a nucleic acid sequence 182 for a guide RNA (gRNA)-target RNA scaffold formed between the gRNA and the target RNA when the gRNA hybridizes to the target RNA, the first encoder block 134-1 (i) receives, as input 140-1 to the first encoder block, a first portion of the nucleic acid sequence 182 for the gRNA-target RNA scaffold that corresponds to a sequence of the gRNA, or a representation thereof, and (ii) generates, as output 142-1, a representation of the first portion of the nucleic acid sequence. In some embodiments. In some embodiments, the second encoder block 134-2 (i) receives, as input 140-2 to the second encoder block, a second portion of the nucleic acid sequence 182 for the gRNA-target RNA scaffold that corresponds to a sequence of the target RNA, or a representation thereof, and (ii) generates, as output 142-2, a representation of the second portion of the nucleic acid sequence. In some embodiments, the decoder block 136 comprises a first portion 144 and a second portion 146, where the first portion 144 comprises a first attention mechanism 138-3-1 that receives, as input 148-1, the output from the first encoder block 142-1 and a second attention mechanism 138-3-2 that receives, as input 148-2, the output from the second encoder block 142-2. In some embodiments, the model generates, as output 184 from the model, a predicted set of one or more metrics for the efficiency or specificity of deamination of the target nucleotide position in the target RNA by the deamination enzyme (e.g., the ADAR protein) when facilitated by hybridization of the test gRNA to the target RNA. In some embodiments, the output from the model comprises any of the outputs disclosed elsewhere herein, including, but not limited to, any of the embodiments for one or more metrics for an efficiency or specificity of deamination of a target nucleotide position in a target RNA by a deamination enzyme (e.g., an ADAR protein) when facilitated by hybridization of a gRNA to the target RNA (see, e.g., the section entitled “Retraining models,” below).

In some embodiments, each of the first encoder block and the second encoder block comprises: a first portion and a second portion, where the first portion comprises an attention mechanism that receives, as input, the corresponding portion of the nucleic acid sequence for the gRNA-target RNA scaffold, or the representation thereof.

In some embodiments, the plurality of parameters in the model comprises at least 500,000 parameters, at least 1×10⁶ parameters, at least 1×10⁷ parameters, at least 1×10⁸ parameters, at least 1×10⁹ parameters, at least 1×10¹⁰ parameters, at least 1×10¹¹ parameters, or at least 2×10¹¹ parameters. In some embodiments, the plurality of parameters in the model comprises no more than 1×10¹² parameters, no more than 1×10¹¹ parameters, no more than 1×10¹⁰ parameters, no more than 1×10⁹ parameters, no more than 1×10⁸ parameters, no more than 1×10⁷ parameters, or no more than 1×10⁶ parameters. In some embodiments, the plurality of parameters in the model consists of from 500,000 to 1×10⁷ parameters, from 1×10⁶ to 1×10⁸ parameters, from 1×10⁸ to 1×10¹⁰ parameters, or from 1×10¹⁰ to 1×10¹² parameters. In some embodiments, the plurality of parameters in the model falls within another range starting no lower than 500,000 parameters, and ending no higher than from 1×10¹² parameters.

In some embodiments, a respective block (e.g., a first block and/or a second block, a respective encoder block and/or a respective decoder block) of the model comprises at least 100,000 parameters, at least 500,000 parameters, at least 1×10⁶ parameters, at least 1×10⁷ parameters, at least 1×10⁸ parameters, at least 1×10⁹ parameters, at least 1×10¹⁰ parameters, or at least 1×10¹¹ parameters. In some embodiments, a respective block comprises no more than 5×10¹¹ parameters, no more than 1×10¹¹ parameters, no more than 1×10¹⁰ parameters, no more than 1×10⁹ parameters, no more than 1×10⁸ parameters, no more than 1×10⁷ parameters, no more than 1×10⁶ parameters, or no more than 500,000 parameters. In some embodiments, a set of parameters in a respective block consists of from 100,000 to 1×10⁷ parameters, from 1×10⁶ to 1×10⁸ parameters, from 1×10⁸ to 1×10¹⁰ parameters, or from 1×10¹⁰ to 5×10¹¹ parameters. In some embodiments, a set of parameters in a respective block falls within another range starting no lower than 100,000 parameters, and ending no higher than from 5×10¹¹ parameters.

### Pretraining Models.

In some embodiments, all or a portion of the model is pretrained. In some embodiments, where the model comprises at least a first block and a second block, at least the first block (e.g., an encoder block is pretrained). In some embodiments, where the model comprises one or more encoder blocks and one or more decoder blocks, the one or more encoder blocks of the model is pre-trained.

In some embodiments, one or more parameters of the model (e.g., for a first block, a second block, an encoder block, and/or a decoder block) reflects pretraining information for a plurality of pretraining samples.

Referring again to Block 202, in some embodiments, the plurality of parameters reflects, at least in part, pretraining information for a plurality of pretraining samples comprising, for each respective pretraining sample in the plurality of pretraining samples, a corresponding unlabeled nucleic acid sequence.

In some embodiments, the model comprises RNA-FM, RNA-MSM, Atom-1, and/or BigRNA. In some embodiments, the pretrained portion of the model comprises any of the models disclosed above, including, but not limited to, RNA-FM, RNA-MSM, Atom-1, and/or BigRNA.

RNA-FM is an RNA foundation model that is based on the BERT language model architecture. It comprises 12 transformer-based bidirectional encoder blocks and is trained on 23 million sequences from the RNAcentral database in a self-supervised manner. After training, RNA-FM produces a L×640 embedding matrix for each RNA sequence with length L. These embeddings are expected to contain rich information within the non-coding RNA (ncRNA) universe. Trained in such manner, RNA-FM embeddings infer functional and structural characteristics of ncRNAs, as well as evolutionary trends of long non-coding RNA (lncRNA), suggesting that the model implicitly learns the functional, structural, and evolutionary information of ncRNAs. See, e.g., Chen et al., “Interpretable RNA Foundation Model from Unannotated Data for Highly Accurate RNA Structure and Function Predictions,” bioRxiv 2022, doi: 10.1101/2022.08.06.503062, which is hereby incorporated herein by reference in its entirety.

RNA-MSM is an unsupervised multiple sequence alignment-based RNA language model that is developed by utilizing homologous sequences from an automatic pipeline. RNA-MSM produces two-dimensional attention maps and one-dimensional embeddings that represent two-dimensional base pairing probabilities and one-dimensional solvent accessibilities, respectively. Such unsupervised training allows the pretrained model to capture structural information from RNA interactions. See, e.g., Zhang et al., “Multiple sequence-alignment-based RNA language model and its application to structural inference,” bioRxiv 2023, doi: 10.1101/2023.03.15.532863, which is hereby incorporated herein by reference in its entirety.

Atom-1 is an RNA foundation model trained on large quantities of chemical mapping data, enabled by data collection strategies across different experimental conditions, chemical reagents, and sequence libraries. Using small probe neural networks on top of ATOM-1 embeddings, the model has developed rich internal representations of RNA, including secondary structure representation. See, e.g., Boyd et al., “ATOM-1: A Foundation Model for RNA Structure and Function Built on Chemical Mapping Data,” bioRxiv 2023, doi: 10.1101/2023.12.13.571579, which is hereby incorporated herein by reference in its entirety.

BigRNA is a foundation model for RNA biology trained on thousands of genome-matched datasets obtained from RNA sequencing (RNA-seq) data, in particular, BigRNA learns from paired genotype and 128 bp resolution RNA expression data from many individuals. By directly modeling RNA-seq data, it can discover a diverse set of pathogenic non-coding mechanisms. Without any additional training, BigRNA accurately identifies compounds that induce a targeted splicing change, and recovers known approved steric blocking oligonucleotides (SBO) therapies with high specificity. See, e.g., Celaj et al., “An RNA foundation model enables discovery of disease mechanisms and candidate therapeutics,” bioRxiv 2023, doi: 10.1101/2023.09.20.558508, which is hereby incorporated herein by reference in its entirety.

In some embodiments, all or a portion of the model is generated and/or pre-trained de novo using a plurality of pretraining samples. In some embodiments, the method further includes obtaining and/or pretraining a respective encoder block in the model (e.g., the first encoder block and/or the second encoder block) using a plurality of pretraining samples. In some embodiments, the method further includes obtaining and/or pretraining each respective encoder block in the model (e.g., the first encoder block and the second encoder block) using a plurality of pretraining samples. In some embodiments, each respective encoder block in the model (e.g., the first encoder block and the second encoder block) is a pretrained component model.

In some embodiments, the plurality of pretraining samples comprises at least 100,000 pretraining samples, at least 500,000 pretraining samples, at least 1×10⁶ pretraining samples, at least 1×10⁷ pretraining samples, at least 5×10⁷ pretraining samples, at least 1×10⁸ pretraining samples, or at least 1×10⁹ pretraining samples.

In some embodiments, the plurality of pretraining samples comprises no more than 1×10¹⁰ pretraining samples, no more than 1×10⁹ pretraining samples, no more than 1×10⁸ pretraining samples, no more than 1×10⁷ pretraining samples, no more than 1×10⁶ pretraining samples, or no more than 500,000 pretraining samples. In some embodiments, the plurality of pretraining samples consists of from 100,000 to 1×10⁶ pretraining samples, from 1×10⁶ to 1×10⁸ pretraining samples, or from 1×10⁸ to 1×10¹⁰ pretraining samples. In some embodiments, the plurality of pretraining samples falls within another range starting no lower than 100,000 pretraining samples, and ending no higher than from 1×10¹⁰ pretraining samples.

In some embodiments, each respective pretraining sample in the plurality of pretraining samples is an unannotated non-coding ribonucleic acid sequence for a taxon in a plurality of taxa.

In some embodiments, the plurality of taxa comprises at least 1,000, at least 10,000, at least 100,000, at least 500,000, or at least 1×10⁶ taxa. In some embodiments, the plurality of taxa comprises no more than 1×10⁷, no more than 1×10⁶, no more than 500,000, no more than 100,000, or no more than 10,000 taxa. In some embodiments, the plurality of taxa consists of from 1,000 to 50,000, from 10,000 to 500,000, from 100,000 to 1×10⁶, or from 1×10⁶ to 1×10⁷ taxa. In some embodiments, the plurality of taxa falls within another range starting no lower than 1,000 taxa and ending no higher than 1×10⁷ taxa.

Referring again to Block 202, in some embodiments, the model 130 generates, responsive to inputting first test information comprising a respective nucleic acid sequence 182 to the model, an indication 184 of a structure or function associated with the nucleic acid sequence 182, or a representation thereof. In some embodiments, prior to retraining the model, the model further generates, responsive to inputting the first test information, an indication of an evolutionary drift for the respective nucleic acid sequence, or a representation thereof.

In some embodiments, prior to retraining of all or a portion of the model, the plurality of parameters is determined by pretraining the model using unsupervised or self-supervised learning. In some embodiments, all or a portion of the model is trained using unsupervised or self-supervised learning. In some embodiments, the method further includes pretraining a respective encoder and/or decoder block in the model using unsupervised or self-supervised learning. In some embodiments, the method further includes pretraining each respective encoder block and/or each respective decoder block in the model using unsupervised or self-supervised learning.

Self-supervised learning using unlabeled data allows for the extraction of meaningful representations from vast amounts of raw information. Unlike supervised learning, which generally uses labeled data for training, self-supervised learning harnesses the inherent structure and relationships within the data itself to formulate pretext tasks that guide the learning process. This allows models to learn rich and informative representations without the need for manual annotation, making it particularly advantageous in scenarios where labeled data is scarce or expensive to obtain.

Self-supervised learning tasks typically involve designing pretext tasks that require the model to make predictions about the data based on certain transformations or context. These tasks can include predicting missing or masked portions of the data, inferring relationships between different parts of the data, or reconstructing the original data from corrupted versions. In the case of RNA sequences, these pretext tasks can include predicting masked or missing nucleotides, inferring relationships between different segments of the sequence, or reconstructing the original sequence from shuffled or partially corrupted versions. By training on these pretext tasks, the model can learn to capture the underlying structure, semantics, and dependencies present in the data, enabling it to generalize well to downstream tasks with limited labeled data. Furthermore, self-supervised learning for RNA sequences can be enhanced by integrating domain-specific knowledge and constraints into the pretext tasks. In some embodiments, such enhancements include incorporating information about RNA secondary structure, conservation of functional motifs, and/or known biological interactions to guide the learning process. By doing so, in some implementations, the pretrained models not only capture generic features of RNA sequences, but also encode biologically relevant information relevant for downstream tasks such as RNA folding prediction, sequence alignment, and functional annotation.

Unsupervised learning includes approaches where a model is trained on input data without explicit supervision or labeled outputs. Instead, the algorithm seeks to uncover patterns, structure, or relationships within the data. The goal of unsupervised learning is to discover inherent patterns or representations that exist within the data, without the need for human intervention or guidance. Typically, unsupervised learning involves algorithms clustering similar data points together or reducing the dimensionality of the data to reveal underlying structures. Common techniques in unsupervised learning include but are not limited to clustering algorithms like K-means clustering, which groups data points into clusters based on similarities, and dimensionality reduction methods like principal component analysis (PCA), which aims to find a lower-dimensional representation of the data while preserving its essential features. Unsupervised and self-supervised learning are further described, for example, in Bergmann, “What is self-supervised learning?” IBM 2023, available on the Internet at ibm.com/topics/self-supervised-learning, which is hereby incorporated herein by reference in its entirety.

In some embodiments, referring again to the model illustrated in FIGS. 3A-B, the model 130 comprises a first encoder block 134-1, a second encoder block 134-2, and a decoder block 136, where each of the first encoder block and the second encoder block is pretrained on a respective plurality of pretraining samples comprising unlabeled nucleic acid sequences, and where the pretraining generates, as output from the model, responsive to inputting information comprising a respective nucleic acid sequence to the model, an indication of a structure or function associated with the nucleic acid sequence, or a representation thereof.

In some embodiments, the first test information further comprises structural information for the respective nucleic acid sequence. In some embodiments, the structural information comprises secondary structural features. In some embodiments, the structural information comprises tertiary structural features. In some embodiments, the structural information comprises one or more structural features. In some embodiments, the structural information comprises any of the structural features disclosed elsewhere herein (see, for example, the section entitled “Retraining models,” below). In some embodiments, the nucleic acid sequence for the first test information is a nucleic acid sequence for a gRNA-target RNA scaffold comprising a first portion that corresponds to a nucleic acid sequence for the gRNA, or a representation thereof, and a second portion that corresponds to the nucleic acid sequence for the target RNA, or a representation thereof, and the structural information for the nucleic acid sequence comprises a plurality of structural features formed between the gRNA and the target RNA when the gRNA hybridizes to the target RNA.

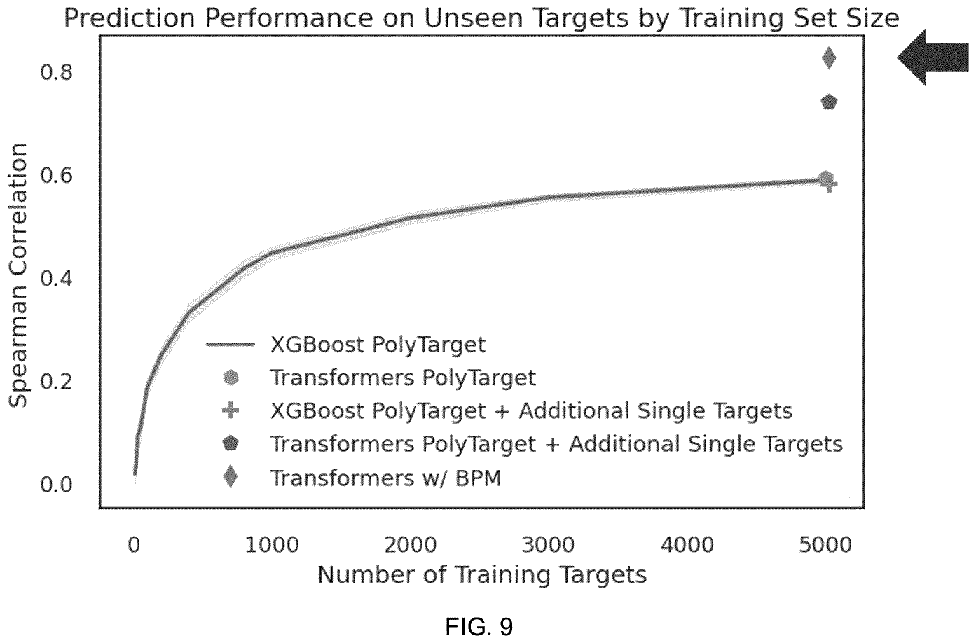

In some embodiments, the structural information comprises, for each respective position in a plurality of positions in the nucleic acid sequence of the first test information, a respective probability that a nucleotide base at the respective position will form a base-pair interaction with a nucleotide base at every other position in the plurality of positions other than the respective position. In some embodiments, the structural information comprises a base-pairing probability matrix (BPM or BPPM). Advantageously, as illustrated in Example 4 and FIG. 9, in some implementations, training and/or using a model by including structural features as inputs improves the predictive output of the model compared to models that are not trained on structural features.

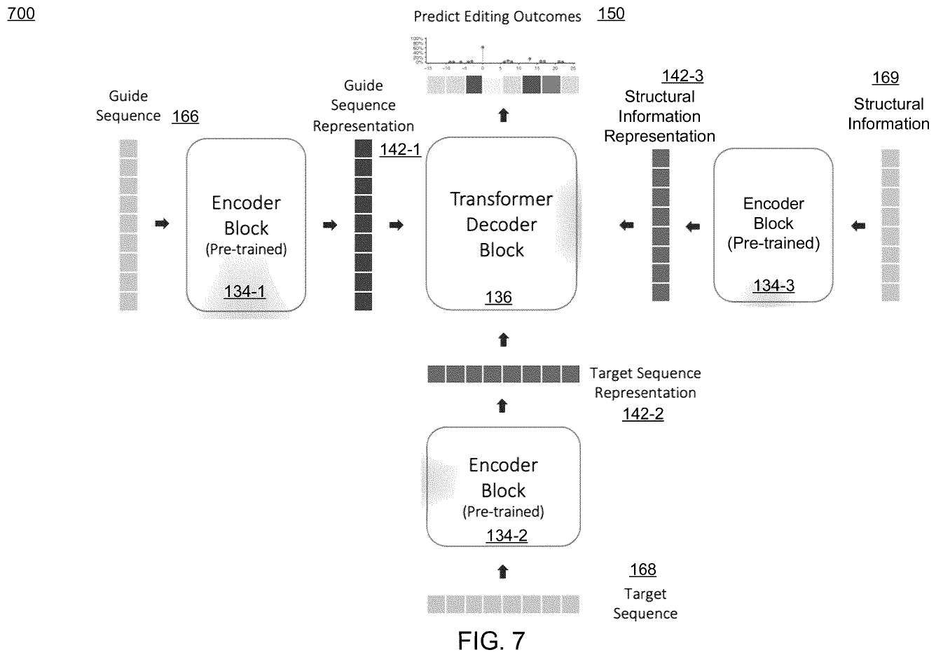

In some embodiments, the structural information is provided as input to one or both of the first block and the second block of the model. In some embodiments, the first block is an encoder block, the second block is a decoder block, and the model receives, responsive to inputting information comprising a corresponding nucleic acid sequence for a gRNA-target RNA scaffold: at an input layer of the first block, (i) a first portion of the nucleic acid sequence for the gRNA-target RNA scaffold that corresponds to the nucleic acid sequence for the gRNA, or a representation thereof, and (ii) all or a portion of the structural information comprising, for each respective position in a plurality of positions in the nucleic acid sequence for the gRNA-target RNA scaffold, a respective probability that a nucleotide base at the respective position will form a base-pair interaction with a nucleotide base at every other position in the plurality of positions other than the respective position. In some embodiments, the model receives, at the input layer of the second block, (i) a second portion of the nucleic acid sequence for the gRNA-target RNA scaffold that corresponds to the nucleic acid sequence for the target RNA, or a representation thereof, and (ii) all or a portion of the structural information comprising, for each respective position in a plurality of positions in the nucleic acid sequence for the gRNA-target RNA scaffold, a respective probability that a nucleotide base at the respective position will form a base-pair interaction with a nucleotide base at every other position in the plurality of positions other than the respective position. In some embodiments, the second block is a second encoder block and the model further includes a decoder block. In some embodiments, the model receives all or a portion of the structural information as input to one or both of the first or the second block. FIG. 7 illustrates, for instance, an example model architecture 700 including a third block 134. The third block is an encoder block 134-3 that, in some implementations, receives, as input, structural information 169 comprising a plurality of structural features for the nucleic acid sequence of a gRNA-target RNA scaffold. In some embodiments, as depicted in FIG. 7, the encoder block generates, as output, a representation of the structural information 142-3.

In some embodiments, the model further comprises a third block, where the third block is an encoder block, and the model receives, responsive to inputting information comprising a corresponding nucleic acid sequence for a gRNA-target RNA scaffold, at an input layer of the third block, the structural information for the corresponding nucleic acid sequence. In some embodiments, the third block comprises an attention mechanism.

In some embodiments, the third block comprises an input layer, and a plurality of hidden layers comprising (i) a first portion that comprises the attention mechanism, and (ii) a second portion. In some embodiments, the second portion of the third block comprises a neural network, a support vector machine, a Naive Bayes model, a nearest neighbor model, a boosted trees model, a random forest model, a decision tree, or a clustering model. In some embodiments, the third block comprises any of the embodiments disclosed herein for a first and/or a second block, as will be apparent to one skilled in the art (see, for example, the section entitled “Example model architecture,” above).

In some embodiments, the model comprises one encoder block. In some embodiments, the model consists of one encoder block and one decoder block. In some embodiments, a respective encoder block in the model receives, as input, at least a nucleic acid sequence (e.g., corresponding to a guide sequence, a target sequence, and/or a gRNA-target RNA scaffold formed between a guide RNA (gRNA) 166 and a target RNA 168 when the gRNA hybridizes to the target RNA. Alternatively or additionally, in some embodiments, a respective encoder block in the model receives, as input, structural information. In some embodiments, the structural information is a plurality of features. In some embodiments, the structural information comprises a base-pairing probability matrix. In some embodiments, the base-pairing probability matrix comprises, for each respective position in a plurality of positions in the nucleic acid sequence of the first test information, a respective probability that a nucleotide base at the respective position will form a base-pair interaction with a nucleotide base at every other position in the plurality of positions other than the respective position. Structural information contemplated for use as input to the model is further described above.

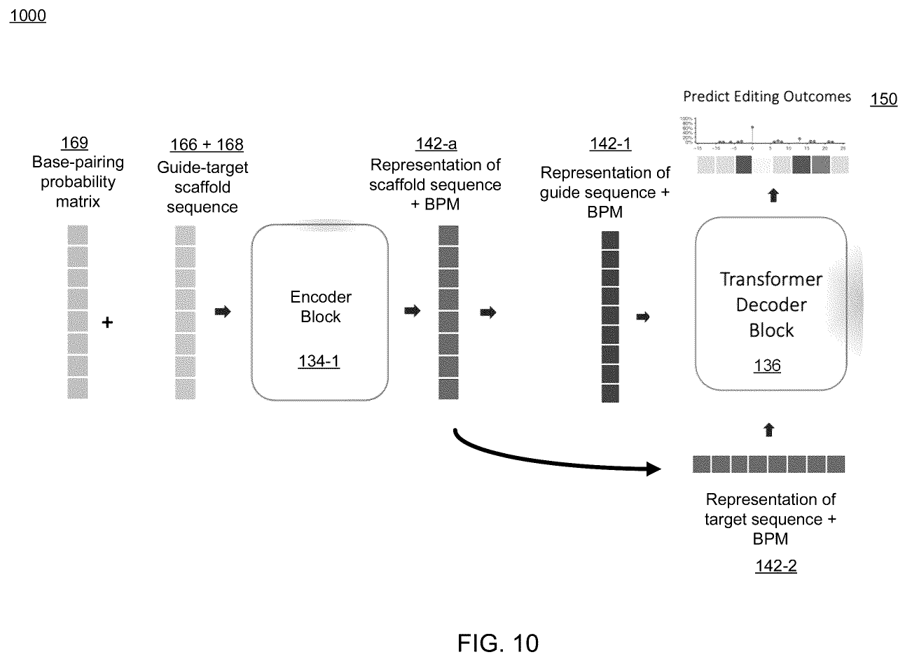

An illustrative schematic of an example model architecture 1000 is further provided in FIG. 10, including an encoder block 134-1 and a decoder block 136. The encoder block receives, as input, a nucleic acid sequence for a gRNA-target RNA scaffold 166+168, as well as structural information in the form of a base-pairing probability matrix 169 (e.g., secondary structure information). The encoder block 134-1 then generates a representation 142-a of the nucleic acid scaffold sequence and the secondary structure information. The representation is then divided into two portions corresponding to the guide RNA 142-1 and the target RNA 142-2. The decoder block 136 receives the two representations 142-1 and 142-2 to generate, as output, a prediction of one or more metrics for an efficiency or specificity of deamination of a target nucleotide position in the target RNA by an Adenosine Deaminase Acting on RNA (ADAR) protein when facilitated by hybridization of the gRNA to the target RNA. In some embodiments, the output from the decoder block comprises any of the outputs disclosed elsewhere herein, including, but not limited to, any of the embodiments for the one or more metrics for the efficiency or specificity of deamination of the target nucleotide position in the target RNA by a deamination enzyme (e.g., an ADAR protein) when facilitated by hybridization of the gRNA to the target RNA (see, e.g., the section entitled “Retraining models,” below).

### Retraining Models.

Referring to Block 204, in some embodiments, the method further includes retraining the model 130 using a plurality of training samples 162, where each respective training sample in the plurality of training samples comprises training information comprising: (i) a corresponding training nucleic acid sequence 164 for a guide RNA (gRNA)-target RNA scaffold formed between the gRNA 166 and the target RNA 168 when the gRNA hybridizes to the target RNA, and (ii) a corresponding training set of one or more metrics 170 for an efficiency or specificity of deamination of a target nucleotide position in the target RNA by an Adenosine Deaminase Acting on RNA (ADAR) protein when facilitated by hybridization of the gRNA 166 to the target RNA 168; thereby updating the plurality of parameters 132.

In some embodiments, the model comprises a first encoder block 134-1, a second encoder block 134-2, and a decoder block 136 and the training updates the plurality of parameters such that the first encoder block 134-1 comprises a first set of parameters 132-1, in the plurality of parameters of the model, that reflects, for each respective training sample 162 in a plurality of training samples, information comprising: (i) a first portion 166 of a respective training nucleic acid sequence for a training scaffold 164, where the training scaffold is formed between a training guide RNA (gRNA) 166 and a target RNA 168 when the training gRNA hybridizes to the target RNA, and where the first portion corresponds to a nucleic acid sequence of the training gRNA, and (ii) a corresponding training set of one or more metrics 170 for an efficiency or specificity of deamination of a target nucleotide position in the target RNA by a deamination enzyme and/or an Adenosine Deaminase Acting on RNA (ADAR) protein when facilitated by hybridization of the training gRNA to the target RNA. In some embodiments, the second encoder block 134-2 comprises a second set of parameters 132-2, in the plurality of parameters of the model, that reflects, for each respective training sample in the plurality of training samples, information comprising: (i) a second portion 168 of the respective training nucleic acid sequence for the training scaffold 164, where the second portion corresponds to the nucleic acid sequence of the target RNA, and (ii) the corresponding training set of one or more metrics 170.

In some embodiments, the gRNA comprises at least 25 nucleotides.

In some embodiments, the gRNA comprises at least 5, at least 10, at least 15, at least 20, at least 25, at least 30, at least 40, at least 50, at least 80, at least 100, at least 120, at least 150, at least 180, or at least 200 nucleotides. In some embodiments, the gRNA comprises no more than 300, no more than 200, no more than 180, no more than 150, no more than 100, no more than 80, no more than 50, no more than 40, no more than 30, no more than 20, or no more than 10 nucleotides. In some embodiments, the gRNA consists of from 5 to 20, from 10 to 40, from 15 to 30, from 25 to 50, from 50 to 100, from 80 to 200, from 100 to 250, or from 120 to 300 nucleotides. In some embodiments, the gRNA falls within another range starting no lower than 5 nucleotides and ending no higher than 300 nucleotides.

In some embodiments, the training information further comprises, for each respective training sample in the plurality of training samples, a nucleic acid sequence for the target RNA comprising a first sub-sequence flanking a 5′ side of a target nucleotide position in the target RNA and a second sub-sequence flanking a 3′ side of the target nucleotide position in the target RNA.

In some embodiments, the plurality of training samples comprises at least 1000 training samples, at least 10,000 training samples, at least 100,000 training samples, at least 500,000 training samples, at least 1×10⁶ training samples, at least 1×10⁷ training samples, or at least 1×10⁸ training samples. In some embodiments, the plurality of training samples comprises no more than 1×10⁹, no more than 1×10⁸, no more than 1×10⁷, no more than 1×10⁶, no more than 500,000, no more than 100,000, or no more than 10,000 training samples. In some embodiments, the plurality of training samples consists of from 1,000 to 50,000, from 10,000 to 500,000, from 100,000 to 1×10⁷, or from 1×10⁶ to 1×10⁹ training samples. In some embodiments, the plurality of training samples falls within another range starting no lower than 1,000 training samples and ending no higher than 1×10⁹ training samples.

In some embodiments, the plurality of training samples is obtained from data generated from in vitro or in vivo experiments, such as from a high-throughput in-cell screening assay.

In some embodiments, the one or more metrics is a plurality of metrics comprising at least 2, at least 3, at least 4, at least 5, at least 10, or at least 20 metrics. In some embodiments, the plurality of metrics comprises no more than 50, no more than 20, no more than 10, no more than 5, or no more than 3 metrics. In some embodiments, the plurality of metrics consists of from 2 to 5, from 2 to 10, from 3 to 15, from 5 to 20, from 8 to 30, or from 20 to 50 metrics. In some embodiments, the plurality of metrics falls within another range starting no lower than 2 metrics and ending no higher than 50 metrics.

In some embodiments, the training set of one or more metrics for the efficiency or specificity of deamination of the target nucleotide position by the deamination enzyme (e.g., ADAR protein) comprises a metric for the efficiency of deamination of the target nucleotide position by the ADAR protein.

In some embodiments, the metric for the efficiency of deamination of the target nucleotide position by a respective deamination enzyme (e.g., ADAR protein) is also referred to interchangeably herein as edit efficiency, editing efficiency, or on-target editing efficiency or score.

In some embodiments, a respective first metric for the efficiency of deamination of the target nucleotide position by a first deamination enzyme (e.g., ADAR protein) is a prevalence of deamination of the target nucleotide position in a plurality of instances of the target RNA.

In some embodiments, a respective first metric for the efficiency of deamination of the target nucleotide position by a first deamination enzyme (e.g., ADAR protein) is a prevalence of the absence of deamination of any nucleotide position in a respective instance of a target RNA in a plurality of instances of the target RNA.

For example, in some embodiments, the prevalence of deamination of the target nucleotide position in a plurality of instances of the target RNA is determined as the proportion of reads with any on-target edits (e.g., an “any on-target editing” metric). In some embodiments, the prevalence of the absence of deamination of any nucleotide position in a respective instance of a target RNA in a plurality of instances of the target RNA is determined as the proportion of reads without any edits (e.g., a “no editing” metric).

In some embodiments, the training set of one or more metrics for the efficiency or specificity of deamination of the target nucleotide position by the deamination enzyme (e.g., ADAR protein) comprises a metric for the specificity of deamination of the target nucleotide position relative to one or more nucleotide positions, other than the target nucleotide position, in the target RNA by the deamination enzyme (e.g., ADAR protein).

In some embodiments, the metric for the specificity of deamination of the target nucleotide position relative to one or more nucleotide positions, other than the target nucleotide position, in the target RNA by the deamination enzyme (e.g., ADAR protein) is: (i) a comparison of (a) a prevalence of deamination of the target nucleotide position in a plurality of instances of the target RNA and (b) a prevalence of deamination of at least one nucleotide position, other than the target nucleotide position, in a respective instance of the target RNA in a plurality of instances of the target RNA, (ii) a prevalence of deamination of the target nucleotide position, without coincident deamination of one or more nucleotide positions other than the target nucleotide position, in a respective instance of the target RNA in a plurality of instances of the target RNA, and/or (iii) a prevalence of deamination of at least one nucleotide position, other than the target nucleotide position, in a respective instance of the target RNA in a plurality of instances of the target RNA.

For example, in some embodiments, the comparison of (a) a prevalence of deamination of the target nucleotide position in a plurality of instances of the target RNA and (b) a prevalence of deamination of at least one nucleotide position, other than the target nucleotide position, in a respective instance of the target RNA in a plurality of instances of the target RNA is determined as (the proportion of reads with on-target edits+1)/(the proportion of reads with off-target edits+1) (e.g., a “specificity” metric). In some embodiments, the prevalence of deamination of the target nucleotide position, without coincident deamination of one or more nucleotide positions other than the target nucleotide position, in a respective instance of the target RNA in a plurality of instances of the target RNA is determined as the proportion of reads with only on-target edits (e.g., a “target-only editing” metric). In some embodiments, the prevalence of deamination of at least one nucleotide position, other than the target nucleotide position, in a respective instance of the target RNA in a plurality of instances of the target RNA is determined as (1−proportion of reads with any off-target edits) (e.g., a “normalized specificity” metric).

In some embodiments, at the one or more nucleotide positions, other than the target nucleotide position, in the target RNA, deamination results in a non-synonymous codon edit.

In some embodiments, a respective metric in the training set of one or more metrics for the efficiency or specificity of deamination of the target nucleotide position by the deamination enzyme (e.g., ADAR protein) is normalized by a metric for an efficiency or specificity of deamination of one or more nucleotide positions, other than the target nucleotide position, in the target RNA by the deamination enzyme (e.g., ADAR protein).

In some embodiments, the training set of one or more metrics further includes an efficiency or specificity of deamination of each target nucleotide position, in a plurality of target nucleotide positions, in the target RNA by the deamination enzyme (e.g., ADAR protein) when facilitated by hybridization of the gRNA to the target RNA. In some embodiments, the plurality of target nucleotide positions includes at least 2, at least 3, at least 4, at least 5, at least 10, or at least 20 target nucleotide positions. In some embodiments, the plurality of target nucleotide positions includes no more than 50, no more than 20, no more than 10, no more than 5, or no more than 3 target nucleotide positions. In some embodiments, the plurality of target nucleotide positions consists of from 2 to 5, from 2 to 10, from 3 to 15, from 5 to 20, from 8 to 30, or from 20 to 50 target nucleotide positions. In some embodiments, the plurality of target nucleotide positions falls within another range starting no lower than 2 target nucleotide positions and ending no higher than 50 target nucleotide positions. In some embodiments, each respective target nucleotide position in the plurality of target nucleotide positions is an adenosine.

In some embodiments, the training set of one or more metrics further includes an efficiency or specificity of deamination of each nucleotide position, in a plurality of nucleotide positions, in the target RNA by the deamination enzyme (e.g., ADAR protein) when facilitated by hybridization of the gRNA to the target RNA. In some embodiments, the plurality of nucleotide positions includes at least 2, at least 3, at least 4, at least 5, at least 10, or at least 20 nucleotide positions. In some embodiments, the plurality of nucleotide positions includes no more than 50, no more than 20, no more than 10, no more than 5, or no more than 3 nucleotide positions. In some embodiments, the plurality of nucleotide positions consists of from 2 to 5, from 2 to 10, from 3 to 15, from 5 to 20, from 8 to 30, or from 20 to 50 nucleotide positions. In some embodiments, the plurality of nucleotide positions falls within another range starting no lower than 2 nucleotide positions and ending no higher than 50 nucleotide positions. In some embodiments, the plurality of nucleotide positions consists of all of the positions in the target RNA.

In some embodiments, the training set of one or more metrics further includes an efficiency or specificity of deamination of each adenosine in the target RNA by the deamination enzyme (e.g., ADAR protein) when facilitated by hybridization of the gRNA to the target RNA.

In some embodiments, the ADAR protein is human ADAR1 or human ADAR2.

In some embodiments, the training information further comprises, for each respective training sample in the plurality of training samples, a corresponding plurality of structural features of the guide-target RNA scaffold formed between the gRNA and the target RNA when the gRNA hybridizes to the target RNA.

In some embodiments, the corresponding plurality of structural features comprises one or more of a micro-footprint and a macro-footprint.

In some embodiments, the plurality of structural features includes at least 5, at least 10, at least 15, or at least 20 structural features. In some embodiments, the plurality of structural features comprises at least 3, at least 5, at least 10, at least 15, at least 20, at least 30, at least 40, at least 50, at least 80, at least 100, or at least 200 structural features. In some embodiments, the plurality of structural features comprises no more than 500, no more than 200, no more than 100, no more than 50, no more than 30, no more than 20, or no more than 10 structural features. In some embodiments, the plurality of structural features consists of from 3 to 20, from 5 to 50, from 20 to 100, from 15 to 80, from 50 to 200, or from 100 to 500 structural features. In some embodiments, the plurality of structural features falls within another range starting no lower than 3 structural features and ending no higher than 500 structural features.

Moreover, in some embodiments, the plurality of structural features includes secondary structural features, tertiary structures, or a combination thereof.

In some embodiments, the plurality of structural features includes one or more structural features selected from the group consisting of: a structural motif comprising two or more structural features; a presence or absence of a mismatch formed upon binding of the gRNA to the target RNA; a position of a mismatch formed upon binding of the gRNA to the target RNA; a presence or absence of a bulge formed upon binding of the gRNA to the target RNA; a position of a bulge formed upon binding of the gRNA to the target RNA; a size of a bulge formed upon binding of the gRNA to the target RNA; a presence or absence of an internal loop in the gRNA upon binding to the target RNA; a position of an internal loop in the gRNA upon binding to the target RNA; a size of an internal loop in the gRNA upon binding to the target RNA; a presence or absence of an internal loop in the target RNA upon binding to the gRNA; a position of an internal loop in the target RNA upon binding to the gRNA; a size of an internal loop in the target RNA upon binding to the gRNA; a presence or absence of a hairpin in the gRNA upon binding to the target RNA; a position of a hairpin in the gRNA upon binding to the target RNA; a size of a hairpin in the gRNA upon binding to the target RNA; a presence or absence of a hairpin in the target RNA upon binding to the gRNA; a position of a hairpin in the target RNA upon binding to the gRNA; a size of a hairpin in the target RNA upon binding to the gRNA; a presence or absence of a wobble base pair formed upon binding of the gRNA to the target RNA; a position of a wobble base pair formed upon binding of the gRNA to the target RNA; a presence or absence of a barbell upon binding of the gRNA to the target RNA; a position of a barbell upon binding of the gRNA to the target RNA; a size of a barbell upon binding of the gRNA to the target RNA; a presence or absence of a dumbbell upon binding of the gRNA to the target RNA; a position of a dumbbell upon binding of the gRNA to the target RNA; a size of a dumbbell upon binding of the gRNA to the target RNA; a presence or absence of a base paired region formed upon binding of the gRNA to the target RNA; a position of a base paired region formed upon binding of the gRNA to the target RNA; a size of a base paired region formed upon binding of the gRNA to the target RNA; a presence or absence of a base paired region formed upon binding of the gRNA to the target RNA; a position of a base paired region formed upon binding of the gRNA to the target RNA; a size of a base paired region formed upon binding of the gRNA to the target RNA; a presence or absence of a U-deletion formed upon binding of the gRNA to the target RNA; a position of a U-deletion formed upon binding of the gRNA to the target RNA; a size of U-deletion formed upon binding of the gRNA to the target RNA; a coaxial stacking formed upon binding of the gRNA to the target RNA; an adenosine platform formed upon binding of the gRNA to the target RNA; an interhelical packing motif formed upon binding of the gRNA to the target RNA; a triplex formed upon binding of the gRNA to the target RNA; a major groove triple formed upon binding of the gRNA to the target RNA; a minor groove triple formed upon binding of the gRNA to the target RNA; a tetraloop motif formed upon binding of the gRNA to the target RNA; a metal-core motif formed upon binding of the gRNA to the target RNA; a ribose zipper formed upon binding of the gRNA to the target RNA; a kissing loop formed upon binding of the gRNA to the target RNA; and a pseudoknot formed upon binding of the gRNA to the target RNA.

In some embodiments, for each respective training sample in the plurality of training samples, the training information further comprises training structural information comprising a plurality of secondary structural features. In some embodiments, for each respective training sample in the plurality of training samples, the training information further comprises training structural information comprising tertiary structural features.

In some embodiments, the training structural information comprises, for each respective position in a plurality of positions in the corresponding training nucleic acid sequence for the gRNA-target RNA scaffold, a respective probability that a nucleotide base at the respective position will form a base-pair interaction with a nucleotide base at every other position in the plurality of positions other than the respective position. In some embodiments, the training structural information comprises a base-pairing probability matrix. In some embodiments, a base-pairing probability matrix is provided as a vector, or as one or more vectors. For example, in some embodiments, a base-pairing probability matrix is provided as separate inputs (e.g., vectors), for each of the target RNA sequence and the gRNA sequence.

In some embodiments, the training structural information is provided as input to one or both of the first block and the second block of the model. In some embodiments, the first block is an encoder block, the second block is a decoder block, and the model receives, responsive to inputting training information comprising a corresponding training nucleic acid sequence for a gRNA-target RNA scaffold: at an input layer of the first block, (i) a first portion of the training nucleic acid sequence for the gRNA-target RNA scaffold that corresponds to the nucleic acid sequence for the gRNA, or a representation thereof, and (ii) all or a portion of the training structural information comprising, for each respective position in a plurality of positions in the training nucleic acid sequence for the gRNA-target RNA scaffold, a respective probability that a nucleotide base at the respective position will form a base-pair interaction with a nucleotide base at every other position in the plurality of positions other than the respective position, and at the input layer of the second block, (i) a second portion of the training nucleic acid sequence for the gRNA-target RNA scaffold that corresponds to the training nucleic acid sequence for the target RNA, or a representation thereof, and (ii) all or a portion of the training structural information comprising, for each respective position in a plurality of positions in the training nucleic acid sequence for the gRNA-target RNA scaffold, a respective probability that a nucleotide base at the respective position will form a base-pair interaction with a nucleotide base at every other position in the plurality of positions other than the respective position. In some embodiments, the second block is a second encoder block, and the model further includes a decoder block.

In some embodiments, the model further comprises a third block, where the third block is an encoder block, and the model receives, responsive to inputting training information comprising a corresponding training nucleic acid sequence for a gRNA-target RNA scaffold, at an input layer of the third block, the training structural information for the corresponding nucleic acid sequence.

In some embodiments, the third block comprises an attention mechanism. In some embodiments, the third block comprises: an input layer, and a plurality of hidden layers comprising (i) a first portion that comprises the attention mechanism, and (ii) a second portion. In some embodiments, the second portion of the third block comprises a neural network, a support vector machine, a Naive Bayes model, a nearest neighbor model, a boosted trees model, a random forest model, a decision tree, or a clustering model.

In some embodiments, the training information includes any of the embodiments disclosed herein for a first test information or a second test information, as will be apparent to one skilled in the art (see, for example, the sections entitled “Pretraining models” and “Example inputs and outputs”).

Methods for updating model parameters during model training (e.g., retraining) are known in the art. In typical embodiments, updating a plurality of parameters is performed by calculating an error for each respective parameter in the plurality of parameters, where the error for each parameter is determined by calculating a loss based on the model output (e.g., the predicted value—generated or experimental) and the training data (e.g., the expected value or true labels—generated or experimental). Parameters are then updated by adjusting the value based on the calculated loss, thereby training the model. Generally, parameters are updated such that the error is minimized (e.g., according to the loss function). In some embodiments, any one of a variety of algorithms and/or methods are contemplated for use in updating parameters, as will be apparent to one skilled in the art. In some embodiments, the loss function is mean square error, quadratic loss, mean absolute error, mean bias error, hinge, multi-class support vector machine, and/or cross-entropy. In some embodiments, the error is computed in accordance with a gradient descent algorithm and/or a minimization function.

In some embodiments, the retraining is performed using fine-tuning, linear probing, or a combination thereof. In some embodiments, linear probing further comprises freezing a first portion of the plurality of parameters such that the parameters in the first portion of the plurality of parameters is not updated, and parameters in a second portion of the plurality of parameters are updated.

Generally, fine-tuning a machine learning model involves the process of adjusting the parameters of a pre-trained model to adapt it to a specific task or dataset. Typically, this is done by utilizing a smaller dataset that is related to the target task or domain. The process starts by initializing the pre-trained model with its learned parameters and then continuing the training process with the new data. During fine-tuning, the model's weights are updated through backpropagation as it learns from the new dataset. Fine-tuning allows the model to capture nuances and patterns specific to the new task, leveraging the general knowledge it gained from the pre-training phase. This process is particularly useful when dealing with limited amounts of data or when the target task is closely related to the original task the model was trained on. By fine-tuning, the model can achieve better performance and adaptability to the new task, making it a powerful technique in machine learning. See, e.g., Howard and Ruder, “Universal Language Model Fine-tuning for Text Classification,” 2018, arXiv:1801.06146, which is hereby incorporated herein by reference in its entirety.

Linear probing is a technique used in model training to address the issue of sparse gradients, where only a few parameters are updated significantly during each training step. Linear probing involves adjusting the learning rate during training based on the magnitude of the gradients. Specifically, it increases the learning rate for parameters with small gradients and decreases it for parameters with large gradients. This adaptive learning rate scheme allows for more efficient training by focusing updates on parameters that require more adjustment while stabilizing those that are already close to optimal values. Linear probing has been shown to improve the convergence speed and performance of various optimization algorithms, making it a valuable tool in training deep learning models. In some embodiments, retraining further includes freezing one or more parameters during retraining. In some such embodiments, parameters with large gradients are typically kept frozen while those with small gradients are updated. By selectively freezing parameters, the model focuses updates on parameters that require significant adjustment, thus accelerating convergence and enhancing the efficiency of the retraining process. This approach ensures that updates are prioritized according to the specific task, resulting in improved performance and stability of the retrained model. See, e.g., Ye et al., “Freeze then Train: Towards Provable Representation Learning under Spurious Correlations and Feature Noise,” 2022, arXiv:2210.11075, which is hereby incorporated herein by reference in its entirety.

In some embodiments, the retraining adjusts all or a portion of a first set of parameters for a first encoder block and/or all or a portion of a second set of parameters for a second encoder block. In some embodiments, the retraining adjusts all or a portion of a third set of parameters for a decoder block. In some embodiments, the retraining comprises freezing a portion of the respective set of parameters for one or more of a first encoder block, a second encoder block, and a decoder block.

In some embodiments, each respective encoder block in a first encoder block and a second encoder block is retrained (e.g., using fine-tuning and/or linear probing) on a different portion of the training information (e.g., a first portion corresponding to a nucleic acid sequence of a training gRNA in a training scaffold, and a second portion corresponding to a nucleic acid sequence of a training target in a training scaffold), but where the parameters for each of the first encoder block and the second encoder block is updated concurrently (e.g., by backpropagation) by adjusting the value for both the first set of parameters and the second set of parameters based on the calculated loss between the model output and the training data (e.g., training labels). Thus, in some embodiments, the plurality of parameters for the model collectively reflects the training information comprising the training scaffold and a corresponding training set of one or more metrics (e.g., labels).

In some embodiments, the plurality of parameters for the model reflects, for each respective training sample in a plurality of training samples, information comprising: (i) a respective training nucleic acid sequence for a training scaffold, wherein the training scaffold is formed between a training gRNA and the target RNA when the training gRNA hybridizes to the target RNA, and (ii) a corresponding training set of one or more metrics for an efficiency or specificity of deamination of a target nucleotide position in the target RNA by the deamination enzyme (e.g., ADAR protein) when facilitated by hybridization of the training gRNA to the target RNA.

In some embodiments, a deep and narrow approach, a sparse and broad approach, or both, are used in training and/or retraining a model. In some embodiments, the plurality of training samples comprises a first subset of training samples and a second subset of training samples, where each respective training sample in the first subset of training samples corresponds to a first subset of target RNA in a plurality of target RNA, each respective training sample in the second subset of training samples corresponds to a second subset of target RNA in a plurality of target RNA, and the retraining comprises: retraining the model using the first subset of training samples, and retraining the model using the second subset of training samples.

In some embodiments, the first subset of target RNA comprises a greater number of target RNA than the second subset of target RNA. In some embodiments, the first subset of training samples is a sparse and broad training set, and the second subset of training samples is a deep and narrow training set.

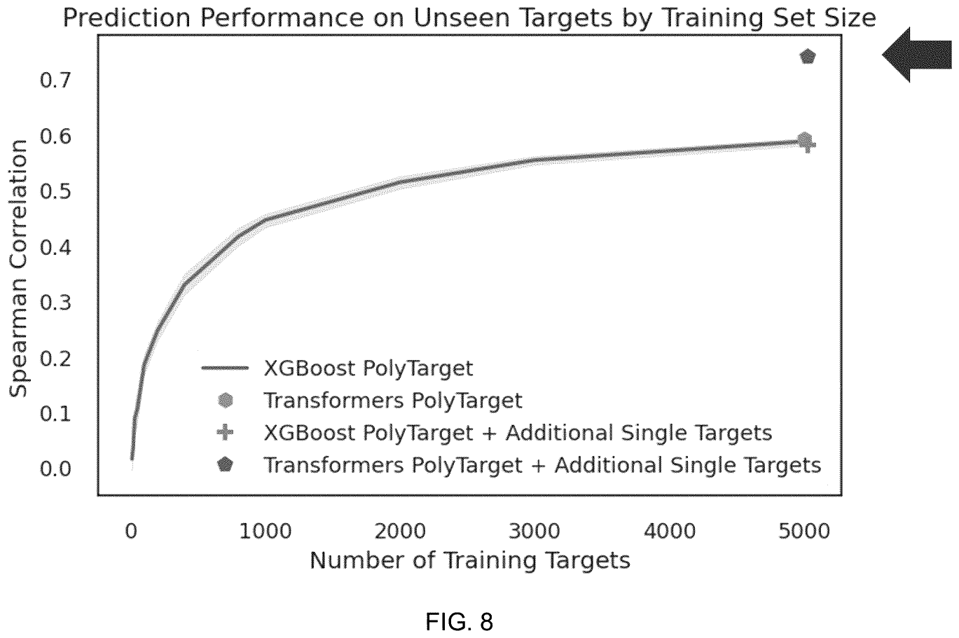

In some embodiments, (i) a number of training samples in the first subset of training samples corresponding to each respective target RNA in the first subset of target RNA is smaller than (ii) a number of training samples in the second subset of training samples corresponding to each respective target RNA in the second subset of target RNA (e.g., where the first subset of training samples is sparse and broad, comprising a relatively sparse number of training samples relative to a relatively large number of target RNA sequences, and where the second subset of training samples is deep and narrow, comprising a relatively deep number of training samples relative to a relatively small number of target RNAs). Advantageously, as illustrated in Example 3 and FIG. 8, in some implementations, training a model using both a sparse and broad and a deep and narrow approach improves the predictive output of a model compared to models that are trained only on a sparse and broad training dataset.

In some embodiments, the first subset of target RNA comprises at least 1000 or at least 5000 target RNA. In some embodiments, the first subset of target RNA comprises at least 100, at least 500, at least 1000, at least 2000, at least 5000, at least 8000, at least 10,000, or at least 20,000 target RNA. In some embodiments, the first subset of target RNA comprises no more than 50,000, no more than 20,000, no more than 10,000, no more than 5000, or no more than 1000 target RNA. In some embodiments, the first subset of target RNA consists of from 100 to 1000, from 500 to 2000, from 2000 to 5000, from 3000 to 10,000, from 8000 to 20,000, or from 20,000 to 50,000 target RNA. In some embodiments, the first subset of target RNA falls within another range starting no lower than 100 target RNA and ending no higher than 50,000 target RNA.

In some embodiments, for each target RNA in the first subset of target RNA, the first subset of training samples comprises from 1 to 10 training samples corresponding to the respective target RNA. In some embodiments, each target RNA in the first subset of target RNA comprises at least 1, at least 10, at least 20, at least 50, at least 100, or at least 500 corresponding training samples. In some embodiments, each target RNA in the first subset of target RNA comprises no more than 1000, no more than 500, no more than 100, no more than 50, or no more than 10 corresponding training samples. In some embodiments, each target RNA in the first subset of target RNA consists of from 1 to 30, from 20 to 100, from 50 to 500, or from 500 to 1000 corresponding training samples. In some embodiments, a number of corresponding training samples for each target RNA in the first subset of target RNA falls within another range starting no lower than 1 training sample and ending no higher than 1000 training samples.

In some embodiments, the second subset of target RNA comprises at least 1, at least 5, or at least 10 target RNA. In some embodiments, the second subset of target RNA comprises at least 1, at least 5, at least 10, at least 20, at least 50, or at least 100 target RNA. In some embodiments, the second subset of target RNA comprises no more than 500, no more than 100, no more than 50, no more than 10, or no more than 5 target RNA. In some embodiments, the second subset of target RNA consists of from 1 to 5, from 2 to 20, from 20 to 50, from 40 to 200, or from 100 to 500 target RNA. In some embodiments, the second subset of target RNA falls within another range starting no lower than 1 target RNA and ending no higher than 500 target RNA.

In some embodiments, for each target RNA in the second subset of target RNA, the second subset of training samples comprises at least 1000 training samples corresponding to the respective target RNA. In some embodiments, each target RNA in the second subset of target RNA comprises at least 100, at least 500, at least 1000, at least 2000, at least 5000, at least 10,000, or at least 20,000 corresponding training samples. In some embodiments, each target RNA in the second subset of target RNA comprises no more than 50,000, no more than 20,000, no more than 10,000, no more than 5000, no more than 2000, no more than 1000, or no more than 500 corresponding training samples. In some embodiments, each target RNA in the second subset of target RNA consists of from 100 to 1000, from 500 to 2000, from 1000 to 10,000, from 5000 to 20,000, or from 20,000 to 50,000 corresponding training samples. In some embodiments, a number of corresponding training samples for each target RNA in the second subset of target RNA falls within another range starting no lower than 100 training samples and ending no higher than 50,000 training samples.

### Example Inputs and Outputs.

In some embodiments, as described above, the model generates, as output, a predicted set of one or more metrics for an efficiency or specificity of deamination of the target nucleotide position in the target RNA by a deamination enzyme (e.g., an ADAR protein) when facilitated by hybridization of the gRNA to the target RNA.

In some embodiments, the method further includes receiving, in electronic form, after the retraining, second test information comprising a nucleic acid sequence for a gRNA-target RNA scaffold formed between the gRNA and the target RNA when the gRNA hybridizes to the target RNA; and inputting the second test information into the retrained model, where the retrained model applies the updated plurality of parameters to the second test information to generate, as output from the retrained model, a test (e.g., predicted) set of one or more metrics for an efficiency or specificity of deamination of the target nucleotide position in the target RNA by the deamination enzyme (e.g., ADAR protein) when facilitated by hybridization of the gRNA to the target RNA.

For instance, in some embodiments, the method further includes, after the retraining (e.g., fine-tuning), using the retrained model to predict a deamination efficiency or specificity for an input gRNA-target RNA scaffold, or a portion thereof.

In some embodiments, the retrained model further generates an estimation of a minimum free energy (MFE) for the gRNA. In some embodiments, the retrained model further generates an estimation of a minimum free energy (MFE) for the guide-target RNA scaffold formed between the guide RNA (gRNA) and the target RNA.

In some embodiments, the output from the retrained model further comprises a metric for an efficiency or specificity of deamination of one or more nucleotide positions, other than the target nucleotide position, in the target RNA by the deamination enzyme (e.g., ADAR protein) when facilitated by hybridization of the gRNA to the target RNA.

In some embodiments, the ADAR protein is human ADAR1 or human ADAR2.

In some embodiments, the output from the retrained model further comprises one or more metrics for an efficiency or specificity of deamination of the target nucleotide position by the deamination enzyme (e.g., ADAR protein) when facilitated by hybridization of the gRNA to the target RNA.

In some embodiments, the output from the retrained model includes any of the metrics disclosed elsewhere herein (see, e.g., the section entitled “Retraining models,” above).

Alternatively or additionally, in some embodiments, the output from the retrained model includes a test (e.g., predicted) set of one or more metrics for an efficiency or specificity of deamination of each target nucleotide position, in a plurality of target nucleotide positions, in the target RNA by the deamination enzyme (e.g., ADAR protein) when facilitated by hybridization of the gRNA to the target RNA. In some embodiments, the plurality of target nucleotide positions includes at least 2, at least 3, at least 4, at least 5, at least 10, or at least 20 target nucleotide positions. In some embodiments, the plurality of target nucleotide positions includes no more than 50, no more than 20, no more than 10, no more than 5, or no more than 3 target nucleotide positions. In some embodiments, the plurality of target nucleotide positions consists of from 2 to 5, from 2 to 10, from 3 to 15, from 5 to 20, from 8 to 30, or from 20 to 50 target nucleotide positions. In some embodiments, the plurality of target nucleotide positions falls within another range starting no lower than 2 target nucleotide positions and ending no higher than 50 target nucleotide positions.

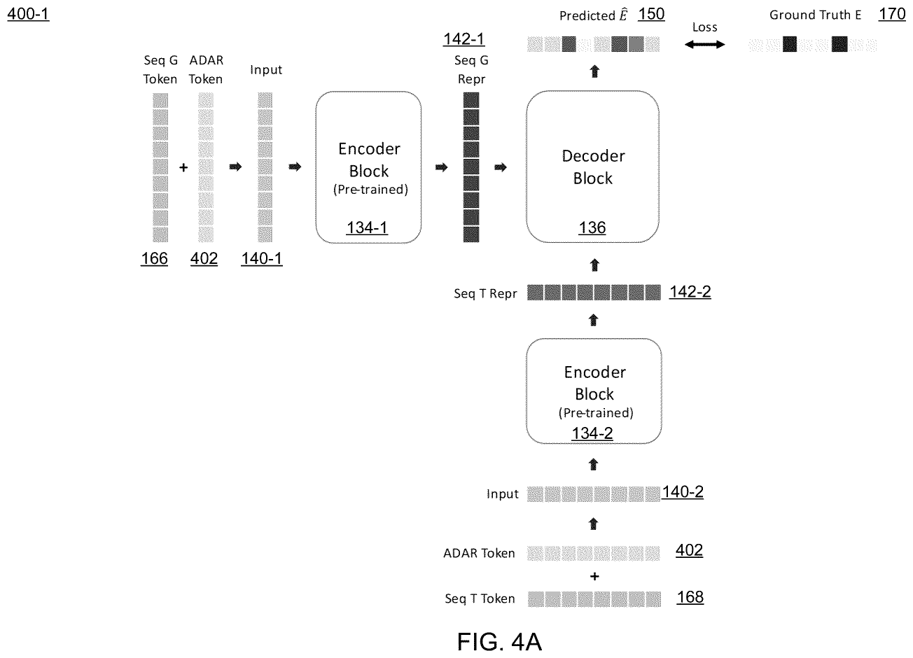

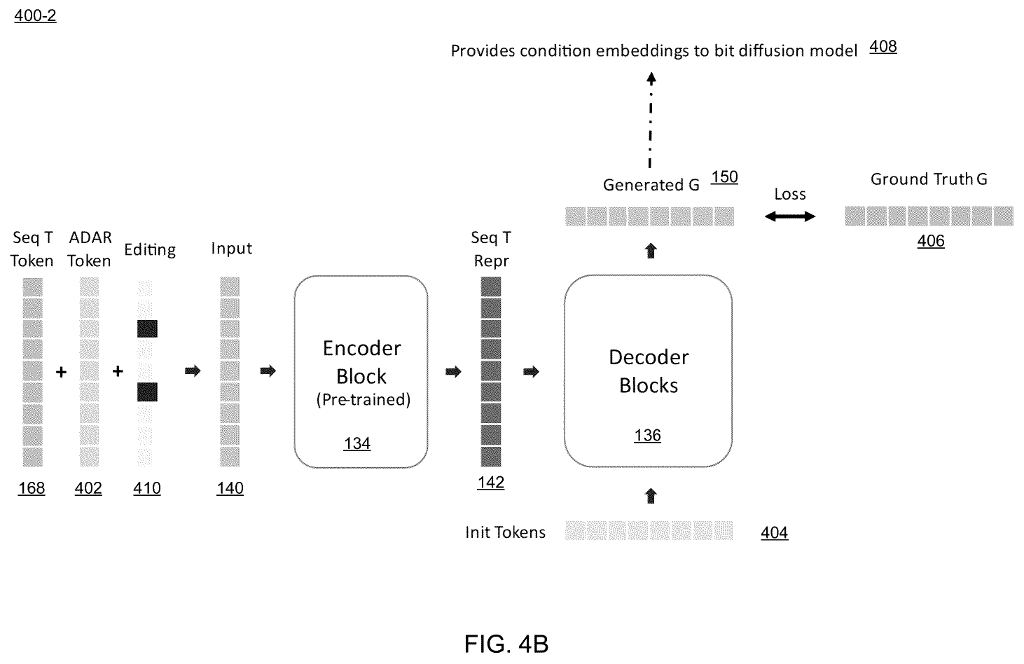

As illustrated in example schematic 400-1 of FIG. 4A, in some embodiments, the retrained model accepts, as input, a nucleic acid sequence for a gRNA-target RNA scaffold comprising a portion corresponding to a gRNA sequence 166 and a portion corresponding to a target RNA sequence 168, and a nucleic acid sequence corresponding to an ADAR protein, or a representation thereof 402. In some embodiments, the input 140-1 to the first encoder block 134-1 comprises the portion of the nucleic acid sequence for the gRNA-target RNA scaffold corresponding to the gRNA sequence 166, or a representation thereof, and the nucleic acid sequence corresponding to the ADAR protein, or the representation thereof 402. In some embodiments, the input 140-2 to the second encoder block 134-2 comprises the portion of the nucleic acid sequence for the gRNA-target RNA scaffold corresponding to the target RNA sequence 168, or a representation thereof, and the nucleic acid sequence corresponding to the ADAR protein, or the representation thereof 402. In some embodiments, the output from the model comprises, as output from the decoder block 150, a predicted deamination efficiency or specificity for the input gRNA-target RNA scaffold. In some embodiments, retraining of the model illustrated in FIG. 4A is performed by adjusting one or more parameters in the plurality of parameters of the model based on a loss between the predicted deamination efficiency or specificity (e.g., the model output) 150 and a corresponding training editing metric, such as training deamination efficiency or specificity (e.g., a label for a training scaffold sequence) 170.

In some embodiments, the method further includes identifying (e.g., selecting) one or more gRNA, from the plurality of gRNA, having, for each respective target nucleotide position in one or more target nucleotide positions, based on a corresponding first metric in the one or more metrics for an efficiency or specificity of deamination of the respective target nucleotide position by the deamination enzyme (e.g., ADAR protein) when facilitated by hybridization of the gRNA to the target RNA.

In some embodiments, the method further includes manufacturing a gRNA comprising the nucleic acid sequence for the selected gRNA that hybridizes to the target RNA for treatment of a disorder. Methods for manufacturing oligonucleotides are well known in the art. For a review of large-scale oligonucleotide synthesis methodologies see, for example, Song L. F., et al., “Large-Scale de novo Oligonucleotide Synthesis for Whole-Genome Synthesis and Data Storage: Challenges and Opportunities,” Frontiers in Bioengineering and Biotechnology, vol. 9 (2021).

In some embodiments, the method further includes administering a gRNA comprising the nucleic acid sequence for the selected gRNA that hybridizes to the target RNA to a subject for treatment of a disorder. That is, administering a gRNA comprising a sequence designed for editing multiple nucleotide positions to a subject having genomic mutations at the multiple nucleotide positions, to edit the nucleotide positions thereby treating a disorder caused at least in part by the mutations of the multiple nucleotide positions.

In some embodiments, the present disclosure provides systems and methods for generating a gRNA using a model trained to predict a deamination efficiency or specificity.

In some embodiments, the methods for generating a gRNA described herein use a model trained to predict the properties of a gRNA, e.g., efficiency, specificity, minimal free energy, etc., for input optimization against a target set of properties. During input optimization, all or a portion of an input construct to the model are updated against a loss function while the parameters of a model are kept fixed. Briefly, an input construct, referred to as an input seed, is input into the model to output a prediction for the properties of the input seed. A loss function is evaluated for a difference between the values of the predicted properties for the input seed and a set of user-defined target property values. This calculated loss is then used to optimize the model over the seed input (or a portion thereof), e.g., using gradient descent or gradient ascent. Unlike machine learning model training, in back-propagation for input optimization, the parameters of the model are kept fixed during this optimization, while the seed inputs are allowed to float.

The optimization is performed over a series of iterations, where in each iteration the seed is input into the model to output predicted values for each gRNA property, the difference between the predicted values and target values is evaluated using a loss function to provide a loss value, and the loss value is used to update the seed using an optimization technique, such as gradient descent and/or gradient ascent. The updated seed is then used as the seed input for the next iteration of the optimization.

In some embodiments, input optimization is performed using an activation maximization process. Methods for input optimization are described, for example, in LeCun, Y., et al., “Handwritten digit recognition with a back-propagation network,” Advances in neural information processing systems, (1989); Simonyan, K., et al., “Deep inside convolutional networks: Visualising image classification models and saliency maps,” arXiv preprint arXiv:1312.6034 (2013); and Selvaraju, R. R., et al., “Grad-CAM: Visual Explanations from Deep Networks Via Gradient-Based Localization,” IEEE International Conference on Computer Vision (ICCV), p. 618-26 (2017), the content of which are incorporated herein by reference in their entireties.

As illustrated in example schematic 400-2 of FIG. 4B, in some embodiments, a retrained model comprises a first block (e.g., an encoder block) 134 and a second block (e.g., a decoder block 136), where the second block accepts, as input, an initialization sequence, or a representation thereof such as initialization tokens 404 (e.g., an input seed). In some embodiments, the first block accepts, as input 140, a nucleic acid sequence for a target RNA sequence 168, or a representation thereof. In some embodiments, the input 140 further comprises a nucleic acid sequence corresponding to the ADAR protein, or a representation thereof 402. In some embodiments, the input further comprises one or more conditions 410, such as a corresponding set of one or more target metrics. In some embodiments, as illustrated in FIG. 4B, the second block generates as output 150 a predicted nucleic acid sequence for a gRNA, or a representation thereof. In some embodiments, the output 150 further comprises the one or more conditions 410, or a representation thereof. In some embodiments, the outputted gRNA sequence, one or more conditions, or representations thereof, as used as inputs 408 to a model for generative model, such as a bit diffusion model. In some embodiments, retraining of the model illustrated in FIG. 4B is performed by adjusting one or more parameters in the plurality of parameters of the model based on a loss between the predicted nucleic acid sequence for the gRNA or representation thereof (e.g., the model output) 150 and a corresponding training gRNA for the input target RNA (e.g., a label for a training target sequence) 406.

### Regulatory Sequences.

In some embodiments, a nucleic acid sequence (e.g., used as an input sequence 140 and/or as a predicted output 150) is a regulatory element or regulatory sequence. In some embodiments, the regulatory element or regulatory sequence is an enhancer or a repressor. In some embodiments, an enhancer is paired with a core promoter to generate a promoter, in which the enhancer enhances transcription of downstream nucleic acid sequence. In some embodiments, a repressor is paired with a core promoter to generate a promoter, in which the repressor represses transcription of a downstream nucleic acid sequence. In some embodiments, an insulator is paired with a core promoter and/or enhancer, in which the insulator modifies trans-activation of the enhancer sequence with the core promoter. In some embodiments, the nucleic acid sequence is all or a portion of a promoter sequence.

In some embodiments, a regulatory element comprises nucleotide sequences, such as promoters, enhancers, terminators, polyadenylation sequences, introns, etc., that provide for the expression of a coding sequence in a cell. In some embodiments, a promoter (alternatively “promoter element”) comprises a DNA regulatory element that coordinates expression of a coding sequence (e.g., RNA transcription). Generally, promoter elements are located 5′ of the translation start site of a gene. In some embodiments, a promoter element is constitutively active (e.g., Jet, CMV, or minCMV) such that it drives effectively constant expression of the coding sequence. In other embodiments, a promoter element is inducible, such that expression of the coding sequence is driven only in the presence of a particular element or condition (either an endogenous or exogenous), such as doxycycline activating of the Tet promoter. In some embodiments, promoters coordinate transcription as part of a cellular genome or as an exogenous element (e.g., a plasmid). In some embodiments, a CMV, CAG, JeT, EFlalpha, TetOn, PGK, MND, or minCMV promoter is used. In some embodiments, a CMV, CAG, JeT, EF1alpha, TetOn, PGK, MND, or minCMV promoter is used to drive expression of a protein coding gene. In some embodiments, a mU7, hU1, or hU6 promoter is used to drive expression of a gRNA.

In some embodiments, the regulatory element comprises a transcriptional termination signal. In some embodiments, a transcriptional termination signal occurs following an open reading frame sequence or other transcriptionally active nucleotide sequence and directs termination of transcription. Optionally, the element recruits other cellular proteins (e.g., polyA polymerase) to the site. This element initiates the process of releasing the newly synthesized RNA from the transcription machinery. Non-exhaustive examples of such elements include an SV40 polyadenylation signal, a bovine growth hormone (BGH) polyadenylation signal, a rabbit beta globin (rbGlob) polyadenylation signal, and a herpes simplex virus type 1 thymidine kinase (HSV TK) polyadenylation signal. The choice of transcriptional termination signals can depend upon the type of cells being used for the screening assay. For example, in some embodiments, prokaryotic cells use rho-dependent and rho-independent transcriptional termination mechanisms, the former of which relies upon formation of GC-rich hairpin in the RNA transcript. In some embodiments, eukaryotic cells also use different transcriptional termination mechanisms, dependent upon the RNA polymerase. For example, Polymerase II, which is primarily responsible for mRNA and miRNA transcription, relies on the recruitment of termination factors resulting in cleavage of the nascent RNA at a cleavage signal positioned between a polyadenylation signal and a GU-rich sequence. These elements are commonly referred to together as a polyadenylation sequence. Polymerase III, which is primarily responsible for expression of tRNA and other short RNA, relies on a specific sequence and RNA secondary structure to induce transcript cleavage, similar to the Rho-independent termination found in prokaryotes.

In some embodiments, the regulatory element comprises a promoter. In some implementations, the promoter directs the activation or inhibition of sequence expression. In some embodiments, the regulatory element comprises an enhancer. In some implementations, enhancers provide transcriptional signals to the promoter (e.g., dependent on context such as cell type and cell state). Enhancers can function through clusters of transcription factor motifs; for instance, in some embodiments, enhancers use varying complexities with respect to the sequence (e.g., vocabulary), organization, and combination (e.g., syntax) of transcription factor motifs. Such complex “grammar” can be difficult to predict. Advantageously, the present disclosure utilizes machine learning approaches to identify enhancer syntax and vocabulary.

In some implementations, the model is a bit diffusion model, and the generated nucleic acid sequence is an enhancer sequence that is conditioned to confer upon the generated enhancer sequence improved performance over endogenous enhancers. In some embodiments, the plurality of target metrics for the one or more target biological properties comprises one or more conditions for the respective enhancer sequence.

In some embodiments, the one or more target biological properties are selected from the group consisting of gene expression activity, cellular activity, relative activity of a first cell type compared to a second cell type, presence or absence of one or more enriched motifs, and/or presence or absence of one or more k-mers. For instance, in some embodiments, the one or more target biological properties includes neuron activity, liver cell activity, and/or cancer cell activity (e.g., mouse primary neuron activity, HepG2 activity, etc., or in a specific cell type (e.g., hepatocyte, neuron, etc.) after in vivo administration, e.g., administration to a mouse or non-human primate). In some embodiments, the plurality of target metrics for the one or more target biological properties comprises a metric determined based on the one or more target biological properties (e.g., a fold change, a log fold change, a mean, a median, and/or a difference between an activity of a first cell type and an activity of a second cell type).

### Modified Nucleotides.

In some embodiments, the machine learning models described herein, e.g., models described above in conjunction with methods 200, 500, and 600 and with respect to FIGS. 2, 5A-B, and 6, consider only natural nucleotides at each position of a polynucleotide sequence, e.g., a gRNA. For example, in some embodiments, a model described herein only allows for adenine (A), cytosine (C), guanine (G), and thymine (T)/uracil (U). Accordingly, in some embodiments, a model described herein allows for any of these four nucleotides to be present at any position in the polynucleotide sequence, e.g., a polynucleotide sequence being evaluated by the model or a polynucleotide sequence being generated de novo in an input optimization process.

In some embodiments, the model is applied such that one or more nucleotide positions in a polynucleotide sequence is limited to only a subset of possible nucleotides. For example, in some embodiments, one or more position in a polynucleotide sequence being generated (e.g., using an input optimization procedure) is fixed as a predetermined nucleotide. In some embodiments, one or more nucleotide positions in a polynucleotide is limited to 1, 2, or 3 possible nucleotides. Said another way, in some embodiments, one or more possible nucleotides is excluded as a possibility at one or more positions of the amino acid sequence being generated. For example, in some embodiments, uracil is excluded as a possible nucleotide at a position of an ADAR gRNA across from a target adenosine residue.

In some embodiments, a model described herein also allows for modified nucleotides to be present at one or more (e.g., all) position of the generated polynucleotide sequence. For example, in some embodiments, a model allows for a 2′-O-methyl (2′-O-Me) base in place of, or in addition to, an unmodified nucleotide of the same base at one or more positions in the polynucleotide sequence.

Exemplary chemical modifications comprise any one of: 5′ adenylate, 5′ guanosine-triphosphate cap, 5′ N7-Methylguanosine-triphosphate cap, 5′ triphosphate cap, 3′ phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, 2-O-methyl 3-phosphorothioate or any combinations thereof.

A chemical modification can be made at any location of the engineered guide RNA. In some cases, a modification may be located in a 5′ or 3′ end. In some cases, a polynucleotide comprises a modification at a base selected from: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150, counted from the 5′ end. More than one modification can be made to the guide RNA. In some cases, a modification can be permanent. In other cases, a modification can be transient. In some cases, multiple modifications may be made to the engineered guide RNA. the engineered guide RNA modification can alter physio-chemical properties of a nucleotide, such as their conformation, polarity, hydrophobicity, chemical reactivity, base-pairing interactions, or any combination thereof.

A chemical modification can also be a phosphorothioate substitute. In some cases, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a polynucleic acid. A modification can also enhance biological activity. In some cases, a phosphorothioate enhanced RNA polynucleic acid can inhibit RNase A, RNase T1, calf serum nucleases, or any combinations thereof. These properties can allow the use of PS-RNA polynucleic acids to be used in applications where exposure to nucleases may be of high probability in vivo or in vitro. For example, phosphorothioate (PS) bonds can be introduced between the last 3-5 nucleotides at the 5′-or 3′-end of a polynucleic acid which can inhibit exonuclease degradation. In some cases, phosphorothioate bonds can be added throughout an entire polynucleic acid to reduce attack by endonucleases.

In some embodiments, chemical modification can occur at 3′OH, group, 5′OH group, at the backbone, at the sugar component, or at the nucleotide base. Chemical modification can include non-naturally occurring linker molecules of interstrand or intrastrand cross links. In one aspect, the chemically modified nucleic acid comprises modification of one or more of the 3′OH or 5′OH group, the backbone, the sugar component, or the nucleotide base, or addition of non-naturally occurring linker molecules. In some embodiments, chemically modified backbone comprises a backbone other than a phosphodiester backbone. In some embodiments, a modified sugar comprises a sugar other than deoxyribose (in modified DNA) or other than ribose (modified RNA). In some embodiments, a modified base comprises a base other than adenine, guanine, cytosine, thymine or uracil. In some embodiments, the engineered guide RNA comprises at least one chemically modified base. In some instances, the engineered guide RNA comprises 2, 3, 4, 5, 6, 7, 8, 9, 10, 15, 20, or more modified bases. In some cases, chemical modifications to the base moiety include natural and synthetic modifications of adenine, guanine, cytosine, thymine, or uracil, and purine or pyrimidine bases.

In some embodiments, the at least one chemical modification of the engineered guide RNA comprises a modification of any one of or any combination of: modification of one or both of the non-linking phosphate oxygens in the phosphodiester backbone linkage; modification of one or more of the linking phosphate oxygens in the phosphodiester backbone linkage; modification of a constituent of the ribose sugar; Replacement of the phosphate moiety with “dephospho” linkers; modification or replacement of a naturally occurring nucleobase; modification of the ribose-phosphate backbone; modification of 5′ end of polynucleotide; modification of 3′ end of polynucleotide; modification of the deoxyribose phosphate backbone; substitution of the phosphate group; modification of the ribophosphate backbone; modifications to the sugar of a nucleotide; modifications to the base of a nucleotide; or stereopure of nucleotide. Chemical modifications to the engineered guide RNA include any modification contained herein, while some exemplary modifications are recited in Table 1.

TABLE 1
Exemplary Chemical Modification.

| Modification of | |
|---|---|
| engineered guide RNA | Examples |
| Modification of one or both | sulfur (S), selenium (Se), BR3 (wherein R can be, e.g., hydrogen, |
| of the non-linking | alkyl, or aryl), C (e.g., an alkyl group, an aryl group, and the like), |
| phosphate oxygens in the | H, NR2, wherein R can be, e.g., hydrogen, alkyl, or aryl, or |
| phosphodiester backbone | wherein R can be, e.g., alkyl or aryl |
| linkage | |
| Modification of one or more | sulfur (S), selenium (Se), BR3 (wherein R can be, e.g., hydrogen, |
| of the linking phosphate | alkyl, or aryl), C (e.g., an alkyl group, an aryl group, and the like), |
| oxygens in the | H, NR2, wherein R can be, e.g., hydrogen, alkyl, or aryl, or |
| phosphodiester backbone | wherein R can be, e.g., alkyl or aryl |
| linkage | |
| Replacement of the | methyl phosphonate, hydroxylamino, siloxane, carbonate, |
| phosphate moiety with | carboxymethyl, carbamate, amide, thioether, ethylene oxide linker, |
| “dephospho” linkers | sulfonate, sulfonamide, thioformacetal, formacetal, oxime, |
| | methyleneimino, methylenemethylimino, methylenehydrazo, |
| | methylenedimethylhydrazo, or methyleneoxymethylimino |
| Modification or | Nucleic acid analog (examples of nucleotide analogs can be found |
| replacement of a naturally | in PCT/US2015/025175, PCT/US2014/050423, |
| occurring nucleobase | PCT/US2016/067353, PCT/US2018/041503, PCT/US18/041509, |
| | PCT/US2004/011786, or PCT/US2004/011833, all of which are |
| | expressly incorporated by reference in their entireties |
| Modification of the ribose- | phosphorothioate, phosphonothioacetate, phosphoroselenates, |
| phosphate backbone | boranophosphates, borano phosphate esters, hydrogen |
| | phosphonates, phosphonocarboxylate, phosphoroamidates, alkyl or |
| | aryl phosphonates, phosphonoacetate, or phosphotriesters |
| Modification of 5′ end of | 5′ cap or modification of 5′ cap —OH |
| polynucleotide | |
| Modification of 3′ end of | 3′ tail or modification of 3′ end —OH |
| polynucleotide | |
| Modification of the | phosphorothioate, phosphonothioacetate, phosphoroselenates, |
| deoxyribose phosphate | borano phosphates, borano phosphate esters, hydrogen |
| backbone | phosphonates, phosphoroamidates, alkyl or aryl phosphonates, or |
| | phosphotriesters |
| Substitution of the | methyl phosphonate, hydroxylamino, siloxane, carbonate, |
| phosphate group | carboxymethyl, carbamate, amide, thioether, ethylene oxide linker, |
| | sulfonate, sulfonamide, thioformacetal, formacetal, oxime, |
| | methyleneimino, methylenemethylimino, methylenehydrazo, |
| | methylenedimethylhydrazo, or methyleneoxymethylimino. |
| Modification of the | morpholino, cyclobutyl, pyrrolidine, or peptide nucleic acid (PNA) |
| ribophosphate backbone | nucleoside surrogates |
| Modifications to the sugar | Locked nucleic acid (LNA), unlocked nucleic acid (UNA), or |
| of a nucleotide | bridged nucleic acid (BNA) |
| Modification of a | 2′-O-methyl, 2′-O-methoxy-ethyl (2′-MOE), 2′-fluoro, 2′- |
| constituent of the ribose | aminoethyl, 2′-deoxy-2′-fuloarabinou-cleic acid, 2′-deoxy, 2′-O- |
| sugar | methyl, 3′-phosphorothioate, 3′-phosphonoacetate (PACE), or 3′- |
| | phosphonothioacetate (thioPACE) |
| Modifications to the base of | Modification of A, T, C, G, or U |
| a nucleotide | |
| Stereopure of nucleotide | S conformation of phosphorothioate or R conformation of |
| | phosphorothioate |

In some embodiments, one or more nucleotide modifications are used to enhance the properties of a polynucleotide. For example, certain 2′-O modifications are known to stabilize polynucleotides in vivo. Moreover, specific patterns of nucleotide modifications are used in certain therapeutic polynucleotides, e.g., to stabilize the polynucleotide in vivo. Accordingly, in some embodiments, the model allows for a predetermined nucleotide modification pattern in the generated nucleic acid sequence. In some embodiments, the model restricts to generated nucleic acid sequence to a predetermined nucleotide modification pattern. Examples of nucleotide modifications patterns that have been used in conjunction with therapeutic polynucleotides, such as ADAR gRNA, are disclosed in U.S. Pat. Nos. 10,988,763; 10,941,402; EP3507366; US20200199586; EP3712269; WO2021071858; WO2021243023; EP2852668; EP3103872; U.S. Pat. Nos. 9,340,784; and 9,796,976, the contents of which are hereby incorporated by reference herein, in their entireties.

### 6.5. Additional Embodiments

Referring to FIGS. 5A-B, another aspect of the present disclosure provides an example method 500 for predicting a deamination efficiency or specificity. In some embodiments, the method is performed at a computer system comprising at least one processor and a memory storing at least one program for execution by the at least one processor. Referring to Block 502, in some embodiments, the method includes obtaining a model 130 comprising a first encoder block 134-1, a second encoder block 134-2, and a decoder block 136.

Referring to Block 504, in some embodiments, the first encoder block 134-1 comprises a first set of parameters 132-1, in a plurality of parameters of the model, that reflects, for each respective training sample 162 in a plurality of training samples, information comprising: (i) a first portion 166 of a respective training nucleic acid sequence for a training scaffold 164, where the training scaffold is formed between a training guide RNA (gRNA) 166 and a target RNA 168 when the training gRNA hybridizes to the target RNA, and where the first portion corresponds to a nucleic acid sequence of the training gRNA, and (ii) a corresponding training set of one or more metrics 170 for an efficiency or specificity of deamination of a target nucleotide position in the target RNA by an Adenosine Deaminase Acting on RNA (ADAR) protein when facilitated by hybridization of the training gRNA to the target RNA. Referring to Block 506, in some embodiments, the second encoder block 134-2 comprises a second set of parameters 132-2, in the plurality of parameters of the model, that reflects, for each respective training sample in the plurality of training samples, information comprising: (i) a second portion 168 of the respective training nucleic acid sequence for the training scaffold 164, where the second portion corresponds to the nucleic acid sequence of the target RNA, and (ii) the corresponding training set of one or more metrics 170. Referring to Block 508, in some embodiments, the decoder block 136 comprises a first portion 144 and a second portion 146, where the first portion 144 comprises a first attention mechanism 138-3-1 that receives, as input 148-1, an output from the first encoder block 142-1 and a second attention mechanism 138-3-2 that receives, as input 148-2, an output from the second encoder block 142-2.

Referring to Block 510, in some embodiments, the method further includes inputting, into the model 130, information comprising a nucleic acid sequence 182 for a test scaffold formed between a test gRNA and the target RNA when the test gRNA hybridizes to the target RNA. Referring to Block 512, in some embodiments, the method further includes receiving, as output 184 from the model 130, a predicted set of one or more metrics for the efficiency or specificity of deamination of the target nucleotide position in the target RNA by the ADAR protein when facilitated by hybridization of the test gRNA to the target RNA.

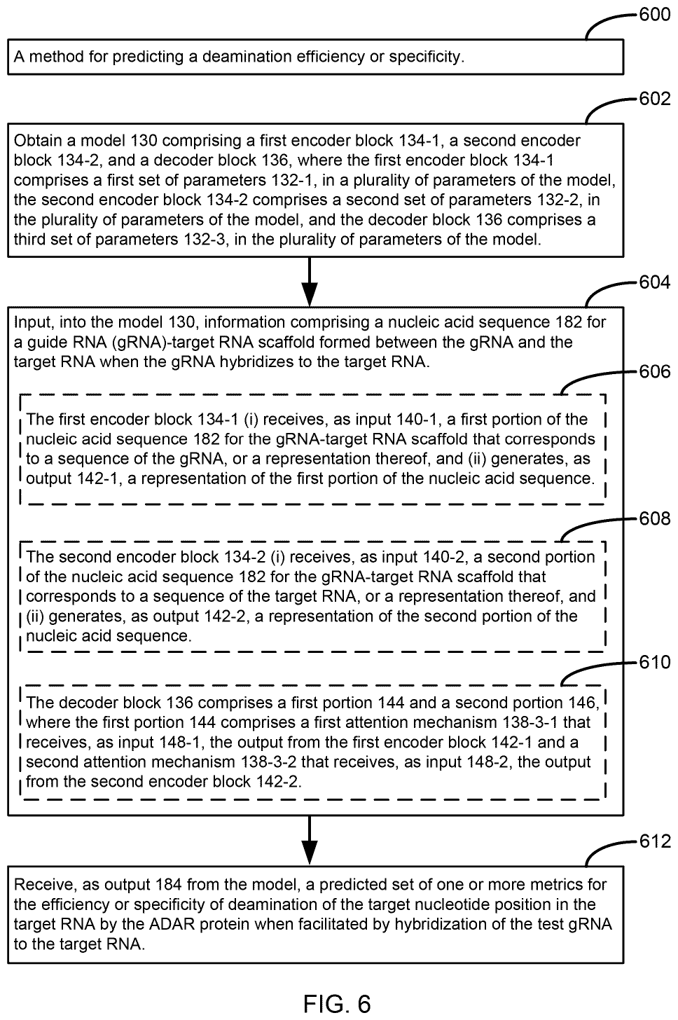

Referring to FIG. 6, another aspect of the present disclosure provides an example method 600 for predicting a deamination efficiency or specificity. In some embodiments, the method is performed at a computer system comprising at least one processor and a memory storing at least one program for execution by the at least one processor. Referring to Block 602, in some embodiments, the method includes obtaining a model 130 comprising a first encoder block 134-1, a second encoder block 134-2, and a decoder block 136, where the first encoder block 134-1 comprises a first set of parameters 132-1, in a plurality of parameters of the model, the second encoder block 134-2 comprises a second set of parameters 132-2, in the plurality of parameters of the model, and the decoder block 136 comprises a third set of parameters 132-3, in the plurality of parameters of the model.

Referring to Block 604, in some embodiments, the method further includes inputting, into the model 130, information comprising a nucleic acid sequence 182 for a guide RNA (gRNA)-target RNA scaffold formed between the gRNA and the target RNA when the gRNA hybridizes to the target RNA. Referring to Block 606, in some embodiments, the first encoder block 134-1 (i) receives, as input 140-1, a first portion of the nucleic acid sequence 182 for the gRNA-target RNA scaffold that corresponds to a sequence of the gRNA, or a representation thereof, and (ii) generates, as output 142-1, a representation of the first portion of the nucleic acid sequence. Referring to Block 608, in some embodiments, the second encoder block 134-2 (i) receives, as input 140-2, a second portion of the nucleic acid sequence 182 for the gRNA-target RNA scaffold that corresponds to a sequence of the target RNA, or a representation thereof, and (ii) generates, as output 142-2, a representation of the second portion of the nucleic acid sequence. Referring to Block 610, in some embodiments, the decoder block 136 comprises a first portion 144 and a second portion 146, where the first portion 144 comprises a first attention mechanism 138-3-1 that receives, as input 148-1, the output from the first encoder block 142-1 and a second attention mechanism 138-3-2 that receives, as input 148-2, the output from the second encoder block 142-2.

Referring to Block 612, in some embodiments, the method further includes receiving, as output 184 from the model, a predicted set of one or more metrics for the efficiency or specificity of deamination of the target nucleotide position in the target RNA by the ADAR protein when facilitated by hybridization of the test gRNA to the target RNA.

Yet another aspect of the present disclosure provides a method for optimizing a model to predict a deamination efficiency of specificity, comprising, at a computer system comprising at least one processor and a memory storing at least one program for execution by the at least one processor: obtaining a model comprising a plurality of parameters across a plurality of blocks, where each of the plurality of blocks comprises an attention mechanism, and the model generates, responsive to inputting first test information comprising a respective nucleic acid sequence to the model, an indication of a structure or function associated with the nucleic acid sequence, or a representation thereof.

In some embodiments, the method further includes obtaining a plurality of training samples, each respective training sample in the plurality of training samples including training information comprising (i) a corresponding training nucleic acid sequence for a guide RNA (gRNA)-target RNA scaffold formed between the gRNA and the target RNA when the gRNA hybridizes to the target RNA, and (ii) a corresponding training set of one or more metrics for an efficiency or specificity of deamination of a target nucleotide position in the target RNA by an Adenosine Deaminase Acting on RNA (ADAR) protein when facilitated by hybridization of the gRNA to the target RNA.

In some embodiments, the method further includes training the model in a first training procedure using a first subset of training samples in the plurality of training samples, each respective training sample in the first subset of training samples corresponding to a first subset of target RNA in a plurality of target RNA. In some embodiments, the method further includes training the model in a second training procedure using a second subset of training samples in the plurality of training samples, each respective training sample in the second subset of training samples corresponding to a second subset of target RNA in a plurality of target RNA, where the first subset of target RNA comprises a greater number of target RNA than the second subset of target RNA, thereby updating the plurality of parameters.

In some embodiments, the first subset of training samples is a sparse and broad training set, and the second subset of training samples is a deep and narrow training set. In some embodiments, (i) a number of training samples in the first subset of training samples corresponding to each respective target RNA in the first subset of target RNA is smaller than (ii) a number of training samples in the second subset of training samples corresponding to each respective target RNA in the second subset of target RNA.

In some embodiments, the first subset of target RNA comprises at least 1000 or at least 5000 target RNA. In some embodiments, for each target RNA in the first subset of target RNA, the first subset of training samples comprises from 1 to 10 training samples corresponding to the respective target RNA. In some embodiments, the second subset of target RNA comprises at least 1, at least 5, or at least 10 target RNA. In some embodiments, for each target RNA in the second subset of target RNA, the second subset of training samples comprises at least 1000 training samples corresponding to the respective target RNA.

In some embodiments, the plurality of blocks comprises a first block and a second block, the model further comprises an output layer, and each of the first block and second block comprises: an input layer, and a plurality of hidden layers comprising (i) a first portion that comprises the attention mechanism, and (ii) a second portion. In some embodiments, the first block is a first encoder block, the second block is second encoder block, the model further comprises a decoder block, and the model receives, responsive to inputting information comprising a corresponding nucleic acid sequence for a gRNA-target RNA scaffold: at the input layer of the first block, a portion of the nucleic acid sequence for the gRNA-target RNA scaffold that corresponds to the nucleic acid sequence for the gRNA, or a representation thereof, and at the input layer of the second block, a portion of the nucleic acid sequence for the gRNA-target RNA scaffold that corresponds to the nucleic acid sequence for the target RNA, or a representation thereof.

In some embodiments, the model further comprises a third block, where the third block is an encoder block, and the model receives, responsive to inputting information comprising a corresponding nucleic acid sequence for a gRNA-target RNA scaffold, at an input layer of the third block, the structural information for the corresponding nucleic acid sequence.

Another aspect of the present disclosure provides a computer system including one or more processors and a non-transitory computer-readable medium including computer-executable instructions that, when executed by the one or more processors, cause the processors to perform any of the methods and/or embodiments disclosed herein.

Yet another aspect of the present disclosure provides a non-transitory computer-readable storage medium having stored thereon program code instructions that, when executed by a processor, cause the processor to perform any of the methods and/or embodiments disclosed herein.

### 6.6. Example Systems and Methods for Predicting Deamination Efficiency or Specificity at Target Nucleotide Positions of Target RNA

Referring to FIGS. 13A-B, another aspect of the present disclosure provides an example method 1300 for predicting a deamination efficiency or specificity at one or more target nucleotide positions 186 of a target RNA. In some embodiments, the method is performed at a computer system comprising at least one processor and a memory storing at least one program for execution by the at least one processor. Referring to Block 1302, in some embodiments, method 1300 includes inputting information about a target-guide scaffold 164 formed between a guide RNA (gRNA) and the target RNA when the gRNA hybridizes to the target RNA into a model 130 to receive as output 184 from the model 130 a predicted set of one or more metrics 150 for an efficiency or specificity of deamination of the one or more target nucleotide positions 186 in the target RNA by a deamination enzyme when facilitated by hybridization of the gRNA to the target RNA. In some embodiments, the information comprises a nucleotide sequence of the gRNA 166, a nucleotide sequence of the target RNA 168 comprising the one or more target nucleotide positions 186, and structural information 169 about the target-guide scaffold. In some embodiments, the model 130 comprises: a first portion comprising one or more encoder blocks that attend to a representation of the nucleotide sequence of the gRNA 166, a representation of the nucleotide sequence of the target RNA 168, and a representation of the structural information 169 about the target-guide scaffold to generate one or more embeddings 142; and a second portion comprising one or more decoder blocks 136 that attend to the one or more embeddings 142 to generate the predicted set of one or more metrics 150.

In some embodiments, the structural information comprises a corresponding plurality of structural features of the target-guide scaffold formed between the gRNA and the target RNA when the gRNA hybridizes to the target RNA.

In some embodiments, the corresponding plurality of structural features comprises one or more of a micro-footprint and a macro-footprint. In some embodiments, the structural information comprises a plurality of secondary structural features. In some embodiments, the structural information comprises tertiary structural features.

In some embodiments, the structural information comprises, for each respective position in a plurality of positions in a nucleic acid sequence for the target-guide scaffold, a respective probability that a nucleotide base at the respective position will form a base-pair interaction with a nucleotide base at every other position in the plurality of positions other than the respective position. In some embodiments, the structural information comprises a base-pairing probability matrix.

Non-limiting structural features contemplated for use in the present disclosure are described in further detail elsewhere herein, e.g., in the sections entitled “Example model architecture” and “Retraining models,” above.

In some embodiments, the model comprises a plurality of parameters.

In some embodiments, the method further includes repeating the inputting, for each respective gRNA-target RNA scaffold in a plurality of gRNA-target RNA scaffolds, thereby receiving, for each respective gRNA-target RNA scaffold in the plurality of gRNA-target RNA scaffolds, a corresponding predicted set of one or more metrics for the efficiency or specificity of deamination of the one or more target nucleotide positions in the target RNA by a deamination enzyme when facilitated by hybridization of the gRNA to the target RNA.

In some embodiments, the model further generates an estimation of a minimum free energy (MFE) for the gRNA. In some embodiments, the model further generates an estimation of a minimum free energy (MFE) for the target-guide scaffold formed between the guide RNA (gRNA) and the target RNA. In some embodiments, the output from the model further comprises a metric for an efficiency or specificity of deamination of one or more nucleotide positions, other than the one or more target nucleotide positions, in the target RNA by the deamination enzyme when facilitated by hybridization of the gRNA to the target RNA. In some embodiments, the deamination enzyme is an Adenosine Deaminase Acting on RNA (ADAR protein). In some embodiments, the ADAR protein is human ADAR1 or human ADAR2. In some embodiments, the predicted set of one or more metrics for the efficiency or specificity of deamination of the one or more target nucleotide positions by the deamination enzyme comprises a metric for the efficiency of deamination of the one or more target nucleotide positions by the deamination enzyme.

In some embodiments, the predicted set of one or more metrics for the efficiency or specificity of deamination of the one or more target nucleotide positions by the deamination enzyme comprises a metric for the specificity of deamination of the one or more target nucleotide positions relative to one or more nucleotide positions, other than the one or more target nucleotide positions, in the target RNA by the deamination enzyme.

In some embodiments, a metric for the specificity of deamination of the one or more target nucleotide positions relative to one or more nucleotide positions, other than the target nucleotide position, in the target RNA by the deamination enzyme is selected from the group consisting of: (i) a comparison of (a) a prevalence of deamination of the one or more target nucleotide positions in a plurality of instances of the target RNA and (b) a prevalence of deamination of at least one nucleotide position, other than the one or more target nucleotide positions, in a respective instance of the target RNA in a plurality of instances of the target RNA, (ii) a prevalence of deamination of the one or more target nucleotide positions, without coincident deamination of one or more nucleotide positions other than the one or more target nucleotide positions, in a respective instance of the target RNA in a plurality of instances of the target RNA, and (iii) a prevalence of deamination of at least one nucleotide position, other than the one or more target nucleotide positions, in a respective instance of the target RNA in a plurality of instances of the target RNA.

In some embodiments, at the at least one nucleotide position, other than the target nucleotide position, in the target RNA, deamination results in a non-synonymous codon edit.

In some embodiments, a respective metric in the predicted set of one or more metrics for the efficiency or specificity of deamination of the one or more target nucleotide positions by the deamination enzyme is normalized by a metric for an efficiency or specificity of deamination of one or more nucleotide positions, other than the one or more target nucleotide positions, in the target RNA by the deamination enzyme.

In some embodiments, the predicted set of one or more metrics further includes an efficiency or specificity of deamination of each target nucleotide position, in a plurality of target nucleotide positions, in the target RNA by the deamination enzyme when facilitated by hybridization of the gRNA to the target RNA. In some embodiments, the predicted set of one or more metrics further includes an efficiency or specificity of deamination of each nucleotide position, in a plurality of nucleotide positions, in the target RNA by the deamination enzyme when facilitated by hybridization of the gRNA to the target RNA. In some embodiments, the predicted set of one or more metrics further includes an efficiency or specificity of deamination of each adenosine in the target RNA by the deamination enzyme when facilitated by hybridization of the gRNA to the target RNA.

Non-limiting metrics contemplated for use in the present disclosure are described in further detail elsewhere herein, e.g., in the sections entitled “Example model architectures” and “Retraining models,” above.

In some embodiments, the gRNA comprises at least 25 nucleotides.

In some embodiments, the information further comprises a nucleic acid sequence for the target RNA comprising a first sub-sequence flanking a 5′ side of a target nucleotide position in the target RNA and a second sub-sequence flanking a 3′ side of the target nucleotide position in the target RNA.

In some embodiments, the plurality of gRNA-target RNA scaffolds comprises at least 1000, at least 10,000, at least 100,000, at least 500,000, at least 1×10⁶, at least 1×10⁷, or at least 1×10⁸ gRNA-target RNA scaffolds.

Non-limiting inputs, including target-guide scaffolds, gRNA, and target RNA sequences, contemplated for use in the present disclosure are described in further detail elsewhere herein, e.g., in the sections entitled “Example model architecture” and “Retraining models,” above.

In some embodiments, the model comprises a language model, a transformer model, a large language model (LLM), an encoder, a decoder, an encoder-decoder hybrid model, a generative pre-trained transformer (GPT) model, a Bidirectional Encoder Representations from Transformers (BERT) model, or a multiple sequence alignment (MSA) transformer model. In some embodiments, the model comprises RNA-FM, RNA-MSM, Atom-1, or BigRNA.

In some embodiments, one or more of the first portion of the model and the second portion of the model comprises an attention mechanism. In some embodiments, the attention mechanism is selected from the group consisting of dot product attention, query-key-value attention, Luong attention, and Bahdanau attention.

In some embodiments, the model further comprises an output layer and each of the first portion and the second portion comprises: an input layer, and a plurality of hidden layers comprising (i) a first sub-portion that comprises the attention mechanism, and (ii) a second sub-portion.

In some embodiments, for each of the first portion and the second portion, the corresponding second sub-portion comprises a neural network, a support vector machine, a Naive Bayes model, a nearest neighbor model, a boosted trees model, a random forest model, a decision tree, or a clustering model. In some embodiments, the second sub-portion of the model comprises an extreme gradient boost (XGBoost) model. In some embodiments, the second sub-portion of the model comprises a convolutional or graph-based neural network.

In some embodiments, the model receives, responsive to inputting the information about the target-guide scaffold: at the input layer of the first portion: (i) the nucleotide sequence of the gRNA, or a representation thereof, (ii) the nucleotide sequence of the target RNA comprising the one or more target nucleotide positions, or a representation thereof, and (iii) the structural information about the target-guide scaffold. In some embodiments, the model receives, at the first sub-portion that comprises the attention mechanism: (i) the nucleotide sequence of the gRNA, or the representation thereof, (ii) the nucleotide sequence of the target RNA comprising the one or more target nucleotide positions, or the representation thereof, and (iii) the structural information about the target-guide scaffold.

Non-limiting model architectures, including inputs, outputs, blocks, encoders, decoders, attention mechanisms, representations, embeddings, and/or encodings, contemplated for use in the present disclosure are described in further detail elsewhere herein, e.g., in the sections entitled “Example model architecture,” “Definitions: Model”, “Pretraining models,” “Retraining models,” and “Example inputs and outputs,” above.

Referring to Block 1304, in some embodiments, the method further includes embedding a nucleic acid sequence for the target-guide scaffold prior to inputting to the first portion.

In some embodiments, the one or more embeddings comprises one or more of: a first embedding for the nucleotide sequence of the gRNA, and a second embedding for the nucleotide sequence of the target RNA comprising the one or more target nucleotide positions. In some embodiments, the first portion generates, as output, (i) an embedding for the nucleotide sequence of the gRNA, or the representation thereof, and (ii) an embedding for the nucleotide sequence of the target RNA comprising the one or more target nucleotide positions, or the representation thereof.

In some embodiments, the first portion generates the first embedding for the nucleotide sequence of the gRNA by considering (e.g., accepting as input, processing, and/or attending to) one or more of: (i) the nucleotide sequence of the gRNA, (ii) the nucleotide sequence of the target RNA, and (iii) the structural information about the target-guide scaffold. In some embodiments, the first portion generates the first embedding for the nucleotide sequence of the gRNA by considering (e.g., accepting as input, processing, and/or attending to) only the nucleotide sequence of the target RNA. In some embodiments, the first portion generates the first embedding for the nucleotide sequence of the gRNA by considering (e.g., accepting as input, processing, and/or attending to) the nucleotide sequence of the target RNA and a nucleotide sequence having perfect complementarity to the nucleotide sequence of the target RNA. In some embodiments, the first portion generates the first embedding for the nucleotide sequence of the gRNA by considering (e.g., accepting as input, processing, and/or attending to) the nucleotide sequence of the target RNA, a nucleotide sequence having perfect complementarity to the nucleotide sequence of the target RNA, and structural information about a target-sequence scaffold formed between the target RNA and the nucleotide sequence having perfect complementarity to the nucleotide sequence of the target RNA.

In some embodiments, the first portion generates the second embedding for the nucleotide sequence of the target RNA by considering one or more of: (i) the nucleotide sequence of the gRNA, (ii) the nucleotide sequence of the target RNA, and (iii) the structural information about the target-guide scaffold. In some embodiments, the first portion generates the second embedding for the nucleotide sequence of the target RNA by considering: only the nucleotide sequence of the target RNA; the nucleotide sequence of the target RNA and a nucleotide sequence having perfect complementarity to the nucleotide sequence of the target RNA; and/or the nucleotide sequence of the target RNA, the nucleotide sequence having perfect complementarity to the nucleotide sequence of the target RNA, and structural information about a target-sequence scaffold formed between the target RNA and the nucleotide sequence having perfect complementarity to the nucleotide sequence of the target RNA. In some embodiments, information considered by the first portion of the model to generate the first embedding is the same or different from information considered by the first portion of the model to generate the second embedding.

In some embodiments, the second portion comprises (i) a first attention mechanism that receives, as input, the embedding for the nucleotide sequence of the gRNA, or the representation thereof, and (ii) a second attention mechanism that receives, as input, the embedding for the nucleotide sequence of the target RNA comprising the one or more target nucleotide positions, or the representation thereof.

In some embodiments, the one or more embeddings comprises an embedding for a nucleic acid sequence for the target-guide scaffold, where the nucleic acid sequence of the target-guide scaffold comprises the nucleotide sequence of the gRNA and the nucleotide sequence of the target RNA comprising the one or more target nucleotide positions. In some embodiments, the first portion generates the embedding for the nucleic acid sequence for the target-guide scaffold by considering one or more of: (i) the nucleotide sequence of the gRNA, (ii) the nucleotide sequence of the target RNA, and (iii) the structural information about the target-guide scaffold. In some embodiments, the first portion generates the embedding for the nucleic acid sequence for the target-guide scaffold by considering: only the nucleotide sequence of the target RNA; the nucleotide sequence of the target RNA and a nucleotide sequence having perfect complementarity to the nucleotide sequence of the target RNA; and/or the nucleotide sequence of the target RNA, the nucleotide sequence having perfect complementarity to the nucleotide sequence of the target RNA, and structural information about a target-sequence scaffold formed between the target RNA and the nucleotide sequence having perfect complementarity to the nucleotide sequence of the target RNA.

In some embodiments, the second portion comprises an attention mechanism that receives, as input, the embedding for a nucleotide sequence of the target-guide scaffold.

Referring to Block 1305, in some embodiments, the method further includes using the model to obtain an embeddings dataset, comprising: determining an initial dataset comprising, for each respective sample in a plurality of samples: a corresponding nucleic acid sequence for a gRNA-target RNA scaffold formed between the gRNA and the target RNA when the gRNA hybridizes to the target RNA, and corresponding structural information about the target-guide scaffold. In some embodiments, obtaining the embeddings dataset further includes inputting, to the first portion of the model, for each respective sample in the plurality of samples: (i) a nucleotide sequence of the gRNA, or a representation thereof, (ii) a nucleotide sequence of the target RNA comprising the one or more target nucleotide positions, or a representation thereof, and (iii) the corresponding structural information about the target-guide scaffold. In some embodiments, obtaining the embeddings dataset further includes obtaining, as output from the first portion of the model, for each respective sample in the plurality of samples, a respective embedding comprising: an embedding for the nucleotide sequence of the gRNA, and an embedding for the nucleotide sequence of the target RNA comprising the one or more target nucleotide positions.

Referring to Block 1306, in some embodiments, the method further includes obtaining an augmented embeddings dataset, comprising: adding noise to one or more embeddings in the embeddings dataset, thereby obtaining one or more noised embeddings; and adding the one or more noised embeddings to the embeddings dataset.

Referring to Block 1308, in some embodiments, the augmented embeddings dataset is obtained using noisy student training or noisy student distillation. In some embodiments, the noise comprises one or more of dropout, stochastic depth, and data augmentation.

Referring to Block 1310, in some embodiments, the method further includes using the augmented embeddings dataset to train a generative model, wherein the generative model determines a nucleic acid sequence for a guide RNA.

Referring to Block 1312, in some embodiments, the generative model comprises a bit diffusion model.

Non-limiting embodiments for encoder outputs, including obtaining embeddings, noising, augmentation, noisy student training, generative models, and methods of use thereof, contemplated for use in the present disclosure are described in further detail elsewhere herein, e.g., in the section entitled “Example model architecture,” above.

In some embodiments, the first portion comprises an encoder block, the second portion comprises a decoder block, and the model receives, responsive to inputting information comprising a corresponding nucleic acid sequence for a gRNA-target RNA scaffold: at the input layer of the first portion, a portion of a nucleic acid sequence for the gRNA-target RNA scaffold that corresponds to the nucleic acid sequence for the gRNA, or a representation thereof, and at the input layer of the second portion, a portion of the nucleic acid sequence for the gRNA-target RNA scaffold that corresponds to the nucleic acid sequence for the target RNA, or a representation thereof.

In some embodiments, the model comprises at least 500,000 parameters, at least 1×10⁶ parameters, at least 1×10⁷ parameters, at least 1×10⁸ parameters, at least 1×10⁹ parameters, at least 1×10¹⁰ parameters, at least 1×10¹¹ parameters, or at least 2×10¹¹ parameters.

In some embodiments, the structural information is provided as input to one or both of the first portion and the second portion of the model.

In some embodiments, the first portion comprises an encoder block, the second portion comprises a decoder block, and the model receives, responsive to inputting information comprising a corresponding nucleic acid sequence for a gRNA-target RNA scaffold: at an input layer of the first portion, (i) a first portion of a nucleic acid sequence for the gRNA-target RNA scaffold that corresponds to the nucleic acid sequence for the gRNA, or a representation thereof, and (ii) all or a portion of the structural information comprising, for each respective position in a plurality of positions in the nucleic acid sequence for the gRNA-target RNA scaffold, a respective probability that a nucleotide base at the respective position will form a base-pair interaction with a nucleotide base at every other position in the plurality of positions other than the respective position. In some embodiments, the model further receives, at an input layer of the second portion, (i) a second portion of the nucleic acid sequence for the gRNA-target RNA scaffold that corresponds to the nucleic acid sequence for the target RNA, or a representation thereof, and (ii) all or a portion of the structural information comprising, for each respective position in a plurality of positions in the nucleic acid sequence for the gRNA-target RNA scaffold, a respective probability that a nucleotide base at the respective position will form a base-pair interaction with a nucleotide base at every other position in the plurality of positions other than the respective position.

In some embodiments, the model further comprises a third portion, where the third portion comprises one or more encoder blocks, and the model receives, responsive to inputting information comprising a corresponding nucleic acid sequence for a gRNA-target RNA scaffold, at an input layer of the third portion, the structural information for the corresponding nucleic acid sequence. In some embodiments, the third portion comprises an attention mechanism. In some embodiments, the third portion comprises: an input layer, and a plurality of hidden layers comprising (i) a first sub-portion that comprises the attention mechanism, and (ii) a second sub-portion. In some embodiments, the second sub-portion of the third portion comprises a neural network, a support vector machine, a Naive Bayes model, a nearest neighbor model, a boosted trees model, a random forest model, a decision tree, or a clustering model.

Non-limiting model architectures contemplated for use in the present disclosure are described in further detail elsewhere herein, e.g., in the sections entitled “Example model architecture,” “Definitions: Model”, “Pretraining models,” “Retraining models,” and “Example inputs and outputs,” above.

Another aspect of the present disclosure provides a method for predicting a deamination efficiency or specificity comprising, at a computer system comprising at least one processor and a memory storing at least one program for execution by the at least one processor: A) obtaining a model comprising a first encoder block, a second encoder block, and a decoder block, where: the decoder block comprises a first portion and a second portion, wherein the first portion comprises a first attention mechanism that receives, as input, an output from the first encoder block and a second attention mechanism that receives, as input, an output from the second encoder block; B) inputting, into the model, information comprising a nucleic acid sequence for a target-guide scaffold formed between a gRNA and the target RNA when the gRNA hybridizes to the target RNA; and C) receiving, as output from the model, a predicted set of one or more metrics for the efficiency or specificity of deamination of one or more target nucleotide positions in the target RNA by a deamination enzyme when facilitated by hybridization of the gRNA to the target RNA.

Yet another aspect of the present disclosure provides a method for predicting a deamination efficiency or specificity comprising, at a computer system comprising at least one processor and a memory storing at least one program for execution by the at least one processor: A) obtaining a model comprising a first encoder block, a second encoder block, and a decoder block, where: the first encoder block comprises a first set of parameters, in a plurality of parameters of the model, the second encoder block comprises a second set of parameters, in the plurality of parameters of the model, and the decoder block comprises a third set of parameters, in the plurality of parameters of the model. In some embodiments, the method further includes B) inputting, into the model, information comprising a nucleic acid sequence for a guide RNA (gRNA)-target RNA scaffold formed between the gRNA and the target RNA when the gRNA hybridizes to the target RNA, where: the first encoder block (i) receives, as input, a first portion of the nucleic acid sequence for the gRNA-target RNA scaffold that corresponds to a sequence of the gRNA, or a representation thereof, and (ii) generates, as output, a representation of the first portion of the nucleic acid sequence. In some embodiments, the second encoder block (i) receives, as input, a second portion of the nucleic acid sequence for the gRNA-target RNA scaffold that corresponds to a sequence of the target RNA, or a representation thereof, and (ii) generates, as output, a representation of the second portion of the nucleic acid sequence. In some embodiments, the decoder block comprises a first portion and a second portion, where the first portion comprises a first attention mechanism that receives, as input, the output from the first encoder block and a second attention mechanism that receives, as input, the output from the second encoder block. In some embodiments, the method further includes C) receiving, as output from the model, a predicted set of one or more metrics for the efficiency or specificity of deamination of one or more target nucleotide positions in the target RNA by a deamination enzyme when facilitated by hybridization of the gRNA to the target RNA.

In some embodiments, each of the first encoder block and the second encoder block comprises: a first portion and a second portion, where the first portion comprises an attention mechanism that receives, as input, the corresponding portion of the nucleic acid sequence for the gRNA-target RNA scaffold, or the representation thereof.

Another aspect of the present disclosure provides a method for predicting a deamination efficiency or specificity. In some embodiments, the method is performed at a computer system comprising at least one processor and a memory storing at least one program for execution by the at least one processor.

In some embodiments, the method includes obtaining a model comprising an encoder block and a decoder block, where the encoder block comprises a first set of parameters, in a plurality of parameters of the model and the decoder block comprises a second set of parameters, in the plurality of parameters of the model.

In some embodiments, the method further includes inputting, into the model, (i) information comprising a nucleic acid sequence for a target-guide scaffold formed between a guide RNA (gRNA) and a target RNA when the gRNA hybridizes to the target RNA, where the nucleic acid sequence for the target-guide scaffold includes a first component corresponding to the gRNA and a second component corresponding to the target RNA, and (ii) structural information for the target-guide scaffold comprising a base-pairing probability matrix.

In some embodiments, the encoder block comprises a first attention mechanism that receives the information comprising the nucleic acid sequence for the target-guide scaffold and the structural information for the target-guide scaffold. In some embodiments, the decoder block comprises a corresponding a first sub-portion and a second sub-portion, where the first sub-portion comprises a second attention mechanism and a third attention mechanism, and where the decoder block receives, as input, an output generated from the encoder block.

In some embodiments, the method further includes receiving, as output from the model, a predicted set of one or more metrics for the efficiency or specificity of deamination of one or more target nucleotide positions in the target RNA by a deamination enzyme when facilitated by hybridization of the gRNA to the target RNA.

An exemplary model in accordance with some embodiments of the present disclosure is illustrated in FIG. 11.

In some embodiments, the nucleic acid sequence for the target-guide scaffold comprises all or a portion of the guide-target scaffold. In some embodiments, the nucleic acid sequence for the target-guide scaffold includes at least a portion of the scaffold corresponding to a target portion of the target RNA. In some embodiments, the nucleic acid sequence for the target-guide scaffold includes at least a portion of the scaffold corresponding to a portion of the gRNA to be placed into a delivery vector, such as a payload for deamination of a target nucleotide. In some embodiments, the gRNA comprises at least 25 nucleotides. Any suitable embodiment for target-guide scaffolds and/or nucleic acid sequences thereof are contemplated for use in the present disclosure, as described elsewhere herein (see, for example, the section entitled “Retraining models,” above).

In some embodiments, the nucleic acid sequence for the target-guide scaffold comprises one or more macro-footprint structural features. In some embodiments, the one or more macro-footprint structural features comprises one or more barbells. In some embodiments, the one or more macro-footprint structural features are positioned at one or both ends of the target-guide scaffold inputted to the model. In some embodiments, the one or more macro-footprint structural features are positioned at other than an end of the target-guide scaffold inputted to the model. For instance, in some embodiments, the one or more macro-footprint structural features are internally positioned within the scaffold, as illustrated by scaffold 164 in FIG. 11.

In some embodiments, the nucleic acid sequence for the target-guide scaffold does not include macro-footprint structural features. In some embodiments, scaffolds used for input to the model are exclusive of macro-footprint structural features, for instance, where input scaffold sequences are selected from regions of a target-guide scaffold located between two barbells but do not include barbells.

In some embodiments, the nucleic acid sequence for the target-guide scaffold includes one or more ancillary sequences for the guide RNA, such as a promoter sequence. In some embodiments, the nucleic acid sequence for the target-guide scaffold does not include an ancillary sequence.

Referring again to FIG. 11, in some embodiments, the information comprising the nucleic acid sequence for the target-guide scaffold comprises a tensor having the dimensions l×d, wherein l is a positive integer representing a number of nucleotide positions in the nucleic acid sequence for the target-guide scaffold.

In some embodiments, l is a positive integer from 100 to 300. In some embodiments, the number of nucleotide positions in the nucleic acid sequence for the target-guide scaffold comprises at least 50, at least 100, at least 200, at least 300, or at least 400 positions. In some embodiments, the target-guide scaffold comprises no more than 500, no more than 400, no more than 300, no more than 200, or no more than 100 positions. In some embodiments, the target-guide scaffold consists of from 50 to 200, from 100 to 300, from 200 to 400, or from 100 to 500 positions. In some embodiments, the target-guide scaffold comprises another range of nucleotide positions starting no lower than 50 positions and ending no higher than 500 positions.

In some embodiments, d is a positive integer representing a number of component encoders in the encoder block. In some embodiments, the encoder block comprises a plurality of component encoders, where d is a positive integer from 3 to 40.

In some embodiments, the encoder block comprises at least 1, at least 2, at least 5, at least 10, at least 30, at least 50, or at least 80 component encoders. In some embodiments, the encoder block comprises no more than 100, no more than 80, no more than 50, no more than 30, no more than 10, or no more than 5 component encoders. In some embodiments, the encoder block consists of from 1 to 10, from 5 to 20, from 15 to 50, from 40 to 80, or from 60 to 100 component encoders. In some embodiments, the encoder block comprises another range of component encoders starting no lower than 1 component encoder and ending no higher than 100 component encoders.

In some embodiments, the first attention mechanism is a multi-head attention mechanism comprising a plurality of attention heads. In some embodiments, each component encoder in d corresponds to a respective attention head in the plurality of attention heads. In some embodiments, d corresponds to a number of possible nucleotide identities in the nucleic acid sequence for the scaffold. In some embodiments, d is at least 4, at least 5, or at least 6. In some embodiments, possible nucleotide identities include, but are not limited to, adenine (A), cytosine (C), guanine (G), thymine (T), and/or uracil (U). In some embodiments, the plurality of possible nucleotide identities further includes an unknown nucleotide (N).

In some embodiments, the method further includes, prior to inputting into the information comprising the nucleic acid sequence for the target-guide scaffold into the encoder block, embedding the nucleic acid sequence for the target-guide scaffold using linear mapping and/or matrix multiplication.

In some embodiments, nucleotide sequences are embedded for input into the model by applying a linear mapping and/or matrix multiplication technique that transforms nucleotide identities and/or symbolic representations thereof into one or more tensor formats (e.g., numerical tensors, matrices, and/or vectors). In some example embodiments, each nucleotide identity (e.g., adenine [A], cytosine [C], guanine [G], thymine [T], and/or uracil [U]) is represented as a categorical token and mapped to a dense, fixed-dimensional vector using a trainable embedding matrix. This embedding matrix serves as a linear transformation, where each row corresponds to a learnable tensor associated with a given nucleotide symbol. As a result, an input nucleotide sequence is converted into a matrix of continuous tensor embeddings, preserving positional, sequence, and other biological information, for instance, in a differentiable, high-dimensional space.

In some embodiments, the embedding uses an embedding tensor (e.g., a matrix) comprising a two-dimensional array, where each entry in a first dimension (e.g., columns) corresponds to a tensor (e.g., a vector) representing a specific category (e.g., a position of a nucleotide or amino acid). In some embodiments, the number of entries in the first dimension corresponds to the number of unique categories. In some embodiments, each entry in a second dimension (e.g., rows) represents the dimensionality of the embedding space. Consider, for instance, an example including 100 nucleotide positions (e.g., l positions) and an embedding dimensionality of 10 (e.g., d component encoders and/or m attention heads). In some such embodiments, the matrix will have 100 columns (one for each nucleotide position) and 10 rows (representing the 10-dimensional vector for each nucleotide position). In some embodiments, the model is trained to adjust the values in the embedding tensor through backpropagation. As the model processes data (e.g., nucleotide sequences), the embedding tensor is fine-tuned to capture relationships between positions. For instance, in some implementations, similar nucleotides or sequences that have similar biological functions or patterns are assigned similar embeddings. The trainable nature of the embedding tensor advantageously allows the resulting embeddings to retain complex relationships and dependencies in the input data. In some embodiments, once the embedding tensor is trained, embedding the nucleic acid sequence for the scaffold includes using the embedding matrix to convert a nucleotide sequence into a series of tensor corresponding to the nucleotide positions in the sequence. In some embodiments, the embeddings are then used as input to the model.

In some embodiments, the method further includes, prior to inputting the information comprising the nucleic acid sequence for the target-guide scaffold into the encoder block, encoding the nucleic acid sequence for the target-guide scaffold using positional encoding. In some embodiments, positional encoding advantageously captures information about the order or position of elements in a sequence (e.g., nucleotide positions in a nucleic acid sequence). In some embodiments, the positional encoding is added to one or more embeddings, such as one or more input embeddings obtained using linear mapping, either as a fixed or learned set of values. In some embodiments, the positional encoding comprises sinusoidal functions, where each position in the sequence is associated with a unique vector derived from sine and cosine functions of different frequencies. This ensures that each position is represented by a distinct vector, with the relationship between positions captured through their encoding.

In some embodiments, the positional encoding comprises absolute positional embeddings, where fixed or learnable vectors are added or concatenated to input token embeddings representing elements in the sequence, based on their positions in the sequence. In some embodiments, relative positional embeddings are employed, which encode the positional relationship between pairs of tokens representing elements in the sequence, allowing the model to generalize across varying input lengths by focusing on relative distances rather than absolute position. In some embodiments, relative positional embeddings are integrated directly into the attention mechanism by modifying a dot product of token embeddings to account for positional offsets. In some embodiments, the positional encoding comprises rotary positional embeddings (RoPE). In some embodiments, RoPE comprises incorporating positional information by applying a rotation matrix to the query and key vectors in the attention mechanism. In some embodiments, and without being limited to any one theory of operation, RoPE enables extrapolation to longer sequences and preserves linearity in the relative positional structure. In some embodiments, RoPE modifies a self-attention computation by rotating each embedding vector in multi-dimensional space based on a position index for the respective embedding vector, thereby encoding distance-dependent phase information. Other positional encoding methods are contemplated for use in the present disclosure, including but not limited to complex-valued embeddings, hybrid relative and absolute methods, and/or a combination of any of the positional encoding methods disclosed herein, as will be apparent to one skilled in the art.

In some embodiments, the information comprising the nucleic acid sequence and the base pairing matrix, or representations thereof, are inputted separately into the model. Alternatively or additionally, in some embodiments, the nucleic acid sequence information for the scaffold and the base pairing probability matrix, or representations thereof, are combined prior to inputting into the model. In some embodiments, the combining comprises concatenating one or more tensor representations of the nucleic acid sequence information and the base pairing probability matrix.

In some embodiments, the first attention mechanism is a multi-head attention mechanism comprising a plurality of attention heads.

As discussed above, in some embodiments, multi-head attention mechanisms advantageously enable a model to focus on, or attend to, different parts of an input sequence simultaneously. Without being limited to any one theory of operation, in some embodiments, a model performs an attention operation by computing attention scores between one or more pairs of tokens (e.g., elements) in a sequence to capture dependencies, allowing it to weigh the importance of one token relative to another. In some embodiments, multi-head attention further includes running multiple attention operations in parallel, with each “head” focusing on different aspects of the input. In some embodiments, each attention head operates on different linear projections of the input data, which enables the model to learn various relationships and interactions between tokens in different subspaces.

In some embodiments, a linear projection of a nucleic acid sequence, or a representation thereof (e.g., an input embedding) comprises a transformation of the original input features into a different space using learned transformation tensors, thereby enabling each attention head to focus on different aspects or relationships within the input data. In an example embodiment, input embeddings comprising embeddings of a nucleic acid sequence for a target-guide scaffold are obtained as described above. In some example embodiments, for each respective attention head in a plurality of attention heads of a multi-head attention mechanism, the input embeddings are linearly transformed into three distinct tensors, e.g., Query, Key, and Value. In some embodiments, the transformation is performed using a plurality of transformation tensors (e.g., matrices) comprising a respective transformation tensor for each of the query, key, and value tensors, denoted W_{Q}, W_{K}, and W_{V}. In some embodiments, each respective transformation tensor comprises a plurality of parameters (e.g., weights), where the plurality of parameters is obtained by a training process. In some embodiments, the query, key, and value tensors are obtained using the functions: Q=X·W_{Q}; K=X·W_{K}; and V=X·W_{V}, where X represents the input token embeddings, and the tensors W_{Q}, W_{K}, W_{V} include learnable parameters that project the input embeddings into new spaces suited for querying, keying, and valuing operations in the attention mechanism. In some embodiments, a respective linear projection is of a lower dimension compared to the input embedding. Without being limited to any one theory of operation, the lower dimensionality allows each respective attention head to capture a different set of relationships between tokens, thereby attending to different kinds of dependencies (e.g., relationships and/or patterns in the input embedding) across multiple attention heads.

In some embodiments, a respective attention mechanism is selected from the group consisting of dot product attention, query-key-value attention, Luong attention, and Bahdanau attention.

In some embodiments, the encoder comprises a multi-head attention mechanism, where each respective attention head generates a corresponding output. In some embodiments, the corresponding output from each respective attention head is concatenated as output from the encoder. In some embodiments, the corresponding output from each respective attention head is combined as a respective dimensionality (e.g., d encoders and/or m attention heads) for the output from the encoder.

Additional suitable embodiments for attention mechanisms contemplated for use in the present disclosure are described elsewhere herein (see, for example, the section entitled “Example model architecture,” above).

In some embodiments, the model comprises at least 500,000 parameters, at least 1×10⁶ parameters, at least 1×10⁷ parameters, at least 1×10⁸ parameters, at least 1×10⁹ parameters, at least 1×10¹⁰ parameters, at least 1×10¹¹ parameters, or at least 2×10¹¹ parameters. In some embodiments, the first set of parameters comprises at least 500,000 parameters, at least 1×10⁶ parameters, at least 1×10⁷ parameters, at least 1×10⁸ parameters, at least 1×10⁹ parameters, at least 1×10¹⁰ parameters, at least 1×10¹¹ parameters, or at least 2×10¹¹ parameters. In some embodiments, the second set of parameters comprises at least 500,000 parameters, at least 1×10⁶ parameters, at least 1×10⁷ parameters, at least 1×10⁸ parameters, at least 1×10⁹ parameters, at least 1×10¹⁰ parameters, at least 1×10¹¹ parameters, or at least 2×10¹¹ parameters.

In some embodiments, the plurality of attention heads comprises at least 5, at least 10, or at least 15 attention heads. In some embodiments, the plurality of attention heads consists of from 3 to 40 attention heads. In some embodiments, the encoder comprises at least 1, at least 2, at least 5, at least 10, at least 20, at least 50, or at least 80 attention heads. In some embodiments, the encoder comprises no more than 100, no more than 80, no more than 50, no more than 20, no more than 10, or no more than 5 attention heads. In some embodiments, the encoder comprises a set of attention heads consisting of from 1 to 5, from 3 to 10, from 5 to 20, from 15 to 50, from 30 to 80, or from 60 to 100 attention heads. In some embodiments, the encoder comprises a set of attention heads that falls within another range starting no lower than 1 attention head and ending no higher than 100 attention heads.

In some embodiments, the base-pairing probability matrix comprises dimensions l×l×m, where l is a positive integer representing a number of nucleotide positions in the nucleic acid sequence for the target-guide scaffold. In some embodiments, l is a positive integer from 100 to 300. In some embodiments, the number of nucleotide positions in the nucleic acid sequence for the target-guide scaffold comprises at least 50, at least 100, at least 200, at least 300, or at least 400 positions. In some embodiments, the target-guide scaffold comprises no more than 500, no more than 400, no more than 300, no more than 200, or no more than 100 positions. In some embodiments, the target-guide scaffold consists of from 50 to 200, from 100 to 300, from 200 to 400, or from 100 to 500 positions. In some embodiments, the target-guide scaffold comprises another range of nucleotide positions starting no lower than 50 positions and ending no higher than 500 positions.

In some embodiments, m is positive integer representing a number of attention heads in the encoder block. In some embodiments, the first attention mechanism is a multi-head attention mechanism comprising a plurality of attention heads, where m is a positive integer of from 3 to 40. As described above, in some embodiments, the encoder comprises at least 5, at least 10, or at least 15 attention heads. In some embodiments, the encoder comprises a set of attention heads consisting of from 3 to 40 attention heads.

In some embodiments, the structural information comprises, for each respective attention head in the plurality of attention heads, a corresponding iteration of the base pairing probability matrix, where each respective attention head in the plurality of attention heads in the encoder block attends to the corresponding iteration of the base pairing probability matrix upon input into the encoder block. In an example embodiment, as illustrated in FIG. 11, the base pairing probability matrix comprises dimensions of l×l×m, where l is a number of positions in the guide-target scaffold sequence and m is a number of attention heads in a multi-head attention mechanism in the encoder block. Consider, for example, a base pairing probability matrix of dimensions 200×200×12, where 200 is the number of nucleotide positions in the guide-target scaffold sequence and 12 is the number of attention heads in a multi-head attention mechanism in the encoder block.

In some embodiments, the method further includes padding the nucleic acid sequence for the target-guide scaffold, where the padding comprises adding one or more filler nucleotides to the nucleic acid sequence until the nucleic acid sequence satisfies a threshold number of nucleotide positions. In some embodiments, the number of nucleotides l in the nucleic acid sequence for the target-guide scaffold that is inputted into the encoder comprises the one or more filler nucleotides, for instance, where the information comprising the nucleic acid sequence for the target-guide scaffold comprises a padded nucleic acid sequence.

In some embodiments, the method further includes padding the base pairing probability matrix, where the padding comprises adding one or more filler nucleotides to the base pairing probability matrix until a dimension of the base pairing probability matrix satisfies a threshold number of nucleotide positions. In some embodiments, the dimension of the base pairing probability matrix is the number of nucleotides or a padded number of nucleotides in the nucleic acid sequence for the target-guide scaffold. In some embodiments, a dimension l of the base pairing probability matrix comprises the one or more filler nucleotides, for instance, where the structural information for the target-guide scaffold comprises the base-pairing probability matrix padded such that a dimension l satisfies the threshold number of nucleotide positions.

In some embodiments, the threshold number of positions comprises at least 100 positions. In some embodiments, the threshold number of positions consists of from 100 to 300 positions. In some embodiments, the threshold number of positions comprises at least 50, at least 100, at least 200, at least 300, or at least 400 positions. In some embodiments, the threshold number of positions comprises no more than 500, no more than 400, no more than 300, no more than 200, or no more than 100 positions. In some embodiments, the threshold number of positions consists of from 50 to 200, from 100 to 300, from 200 to 400, or from 100 to 500 positions. In some embodiments, the threshold number of positions comprises another range of positions starting no lower than 50 positions and ending no higher than 500 positions.

Referring again to FIG. 11, in some embodiments, the nucleic acid sequence for the target-guide scaffold further comprises a concatenation junction between the first component corresponding to the gRNA and the second component corresponding to the target RNA, where the padding further comprises adding the one or more filler nucleotides to a 5′ end or a 3′ end of the nucleic acid sequence for the target-guide scaffold such that the padding positions the concatenation junction at a reference position within the nucleic acid sequence for the target-guide scaffold.

In some embodiments, the method further includes inputting, for each respective target-guide scaffold in a plurality of target-guide scaffolds, (i) respective information comprising a nucleic acid sequence for the respective target-guide scaffold, where the nucleic acid sequence for the respective target-guide scaffold comprises a corresponding first component for the gRNA, a corresponding second component for the target RNA, and a corresponding concatenation junction between the first component and the second component; and padding one or more target-guide scaffolds in the plurality of target-guide scaffolds, where, for each respective target-guide scaffold in the plurality of target-guide scaffolds, the padding comprises adding one or more filler nucleotides to a 5′ end or a 3′ end of the nucleic acid sequence for the respective target-guide scaffold such that the padding positions the concatenation junction at a same reference position in the plurality of target-guide scaffolds. In some such embodiments, the corresponding concatenation junction between the first portion and the second portion is located at the same reference position within the nucleotide sequence for the target-guide scaffold as every other target-guide scaffold. In some embodiments, an alignment of the plurality of target-guide scaffolds aligns the corresponding concatenation junction of each respective target-guide scaffold in the plurality of target-guide scaffolds at the same reference position.

In some embodiments, a respective filler nucleotide in the one or more filler nucleotides comprises a symbol for an unknown nucleotide N. In some embodiments, the padding is performed prior to the embedding, where embedding the nucleic acid sequence for the target-guide scaffold and/or embedding the base pairing probability matrix further includes embedding the one or more filler nucleotide symbols (N). In some embodiments, the embedding comprises any of the embedding methods disclosed above, including but not limited to linear mapping and/or matrix multiplication.

In some embodiments, the method further includes generating, as output from the encoder block, an intermediate embedding of the nucleic acid sequence for the target-guide scaffold, where the intermediate embedding comprises a first component intermediate embedding for the gRNA and a second component intermediate embedding for the target RNA.

In some embodiments, the intermediate embedding comprises dimensions l×d, where l is a positive integer representing a number of nucleotide positions in the nucleic acid sequence for the target-guide scaffold. In some embodiments, d is a positive integer representing a number of component encoders in the encoder block. In some embodiments, d is a positive integer representing a number of component decoders in the decoder block.

In some embodiments, the decoder block comprises a plurality of component decoders. In some embodiments, the decoder block comprises at least 1, at least 2, at least 5, at least 10, at least 30, at least 50, or at least 80 component decoders. In some embodiments, the decoder block comprises no more than 100, no more than 80, no more than 50, no more than 30, no more than 10, or no more than 5 component decoders. In some embodiments, the decoder block consists of from 1 to 10, from 5 to 20, from 15 to 50, from 40 to 80, or from 60 to 100 component decoders. In some embodiments, the decoder block comprises another range of component decoders starting no lower than 1 component decoder and ending no higher than 100 component decoders.

In some embodiments, the decoder block comprises a plurality of component decoders, the second attention mechanism is a multi-head attention mechanism comprising a corresponding second plurality of attention heads, and the third attention mechanism is a multi-head attention mechanism comprising a corresponding third plurality of attention heads.

In some embodiments, the second attention mechanism comprises at least 5, at least 10, or at least 15 attention heads. In some embodiments, the second attention mechanism consists of from 3 to 40 attention heads. In some embodiments, the second attention mechanism comprises at least 1, at least 2, at least 5, at least 10, at least 20, at least 50, or at least 80 attention heads. In some embodiments, the second attention mechanism comprises no more than 100, no more than 80, no more than 50, no more than 20, no more than 10, or no more than 5 attention heads. In some embodiments, the second attention mechanism consists of from 1 to 5, from 3 to 10, from 5 to 20, from 15 to 50, from 30 to 80, or from 60 to 100 attention heads. In some embodiments, the second attention mechanism falls within another range starting no lower than 1 attention head and ending no higher than 100 attention heads.

In some embodiments, the third attention mechanism comprises at least 5, at least 10, or at least 15 attention heads. In some embodiments, the third attention mechanism consists of from 3 to 40 attention heads. In some embodiments, the third attention mechanism comprises at least 1, at least 2, at least 5, at least 10, at least 20, at least 50, or at least 80 attention heads. In some embodiments, the third attention mechanism comprises no more than 100, no more than 80, no more than 50, no more than 20, no more than 10, or no more than 5 attention heads. In some embodiments, the third attention mechanism consists of from 1 to 5, from 3 to 10, from 5 to 20, from 15 to 50, from 30 to 80, or from 60 to 100 attention heads. In some embodiments, the third attention mechanism falls within another range starting no lower than 1 attention head and ending no higher than 100 attention heads. In some embodiments, the number of component decoders and the number of attention heads in the second attention mechanism and/or the third attention mechanism are the same or different.

In some embodiments, the third attention mechanism of the first sub-portion of the decoder block receives, as input, a first component embedding for a nucleic acid sequence of the gRNA, and the second attention mechanism of the first sub-portion of the decoder block receives, as input, a second component embedding for a nucleic acid sequence of the target RNA. In some embodiments, where the method comprises generating, as output from the encoder block, an intermediate embedding of the nucleic acid sequence for the target-guide scaffold comprising a first component intermediate embedding for the nucleotide sequence of the gRNA and a second component intermediate embedding for the nucleotide sequence of the target RNA, the second attention mechanism receives, as input, the second component intermediate embedding (e.g., for the target RNA), and the third attention mechanism receives, as input, the first component intermediate embedding (e.g., for the gRNA).

In some embodiments, the second attention mechanism generates, as output, a first intermediate representation of the nucleic acid sequence for the target RNA, and the third attention mechanism further receives, as input, the first intermediate representation of the nucleic acid sequence for the target RNA. In some embodiments, the intermediate representation comprises a further embedding of the nucleotide sequence for the target RNA. In some embodiments, the third attention mechanism of the decoder receives, as input, both the component intermediate embedding for the gRNA and the output of the second attention mechanism comprising the representation for the target RNA. In some embodiments, the third attention mechanism generates, as output, a second intermediate representation corresponding to the target RNA and the gRNA.

In some embodiments, the second sub-portion of the decoder further comprises a position-wise feed-forward network that accepts, as input, an output from the first sub-portion, and generates, as output, the predicted set of one or more metrics for the efficiency or specificity of deamination of the target nucleotide position in the target RNA by the deamination enzyme when facilitated by hybridization of the test gRNA to the target RNA, or a representation thereof. In some embodiments, the position-wise feed-forward network accepts, as input, the second intermediate representation generated using both the target RNA and the gRNA.

In some embodiments, the model further comprises a fully connected layer that accepts, as input, an output from the decoder, thereby generating the predicted set of one or more metrics for the efficiency or specificity of deamination of the target nucleotide position in the target RNA by the deamination enzyme when facilitated by hybridization of the test gRNA to the target RNA.

In some embodiments, the method further includes repeating the inputting, for each respective target-gRNA scaffold in a plurality of gRNA-target scaffolds, thereby receiving, for each respective target-gRNA scaffold in the plurality of target-gRNA scaffolds, a corresponding predicted set of one or more metrics for the efficiency or specificity of deamination of the one or more target nucleotide positions in the target RNA by a deamination enzyme when facilitated by hybridization of the gRNA to the target RNA.

In some embodiments, the model further generates an estimation of a minimum free energy (MFE) for the gRNA. In some embodiments, the model further generates an estimation of a minimum free energy (MFE) for the target-guide scaffold formed between the gRNA and the target RNA. In some embodiments, the output from the model further comprises a metric for an efficiency or specificity of deamination of one or more nucleotide positions, other than the one or more target nucleotide positions, in the target RNA by the deamination enzyme when facilitated by hybridization of the gRNA to the target RNA. In some embodiments, the deamination enzyme is an Adenosine Deaminase Acting on RNA (ADAR protein). In some embodiments, the ADAR protein is human ADAR1 or human ADAR2.

In some embodiments, the predicted set of one or more metrics for the efficiency or specificity of deamination of the one or more target nucleotide positions by the deamination enzyme comprises a metric for the specificity of deamination of the one or more target nucleotide positions relative to one or more nucleotide positions, other than the one or more target nucleotide positions, in the target RNA by the deamination enzyme. In some embodiments, the predicted set of one or more metrics further includes an efficiency or specificity of deamination of each target nucleotide position, in a plurality of target nucleotide positions, in the target RNA by the deamination enzyme when facilitated by hybridization of the gRNA to the target RNA. In some embodiments, the predicted set of one or more metrics further includes an efficiency or specificity of deamination of each adenosine in the target RNA by the deamination enzyme when facilitated by hybridization of the gRNA to the target RNA. Any of suitable embodiments for predicted metrics are contemplated for use in the present disclosure, as described elsewhere herein (see, for example, the section entitled “Retraining models,” above).

### Pharmaceutical Agents and Administration.

In some embodiments, methods disclosed herein further include synthesizing one or more gRNAs. In some embodiments, methods disclosed herein further include synthesizing the gRNA, after receiving the predicted set of one or more metrics for the efficiency or specificity of deamination from the model.

In some embodiments, methods further include validating the synthesized gRNA. In some embodiments, validating the synthesized gRNA comprises using in vitro screening.

As noted above, in some implementations, synthesizing gRNAs after prediction of metrics using the model advantageously improves screening of guides for use in vitro or in vivo, for instance, for use in therapeutic applications. In some implementations, synthesizing gRNAs after prediction of metrics using the model improves the efficiency of guide screening, by reducing the number of potential gRNAs to be used in in vitro screening and by improving the efficiency, specificity, and editing efficacy of synthesized gRNAs compared to gRNAs not generated using in silico prediction or screening methods.

In some embodiments, methods further include placing one or more gRNAs (e.g., the synthesized gRNA) into a delivery vector.

As noted above, in some implementations, delivery of gRNAs via a delivery vector allows for programmable and precise RNA editing of target nucleotide sequences via recruitment of ADAR proteins in a subject. In some embodiments, the synthesized gRNA placed into the delivery vector corresponds to all or a portion of a target nucleotide sequence to be edited.

In some embodiments, the delivery vector comprises a viral vector, including but not limited to an adeno-associated virus (AAV) and/or a lentivirus. In some embodiments, a viral vector system useful for delivery of nucleic acids comprises an adeno-associated virus (AAV). Adeno-associated virus is a naturally occurring defective virus that requires another virus, such as an adenovirus or a herpes virus, as a helper virus for efficient replication and a productive life cycle. See, for instance, Muzyczka et al., Curr. Topics in Micro. and Immunol. 158:97-129 (1992). In some embodiments, the viral vector comprises a lentivirus. In an aspect, the lentivirus is selected from the group consisting of: human immunodeficiency-1 (HIV-1), human immunodeficiency-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

In some embodiments, the delivery vector comprises a lipid nanoparticle (LNP). Without wishing to be bound by any particular theory, in certain embodiments, nucleic acids, when present in a nanoparticle, are resistant in aqueous solution to degradation with a nuclease. In other embodiments, proteins are protected from protease degradation. In some embodiments, proteins and nucleic acids encapsulated by nanoparticles are capable of penetrating the cellular plasma membrane. In some embodiments, the delivery vector comprises a virus-like particle (VLP). Without wishing to be bound by any particular theory, in certain embodiments, nucleic acids, when present in a virus-like particle, are resistant in aqueous solution to degradation with a nuclease. In other embodiments, proteins are protected from protease degradation while present in the particle. In some embodiments, proteins and nucleic acids encapsulated by VLPs are capable of penetrating the cellular plasma membrane. In some embodiments, the delivery vector comprises liposomes and/or lipid nanocrystals (LNC).

In some embodiments, methods disclosed herein further include formulating a pharmaceutical agent comprising one or more gRNAs (e.g., the synthesized gRNA), after receiving the predicted set of one or more metrics for the efficiency or specificity of deamination from the model.

In some embodiments, the pharmaceutical agent is engineered to target a nucleic acid sequence comprising all or a portion of the target RNA corresponding to a respective gRNA. As described above, in some implementations, the pharmaceutical agent targets the target nucleic acid sequence by facilitating ADAR-mediated RNA editing and binding and/or degradation of target RNA. In some implementations, the pharmaceutical agent comprising the gRNA facilitates introduction of mutations at sites targeted by enzymes in order to modify the affinity of such enzymes for targeting and cleaving such sites. In some embodiments, the pharmaceutical agent comprises the gRNA placed within a delivery vector (e.g., any of the delivery vectors disclosed above).

In some embodiments, the pharmaceutical agent further comprises a pharmaceutically acceptable carrier. In some embodiments, therapeutic compounds are prepared with carriers that will protect the therapeutic compounds against rapid elimination from the body, such as a controlled release formulation, including implants and microencapsulated delivery systems. Biodegradable, biocompatible polymers can be used, such as collagen, ethylene vinyl acetate, polyanhydrides (e.g., poly[1,3-bis(carboxyphenoxy)propane-co-sebacic-acid] (PCPP-SA) matrix, fatty acid dimer-sebacic acid (FAD-SA) copolymer, poly(lactide-co-glycolide)), polyglycolic acid, collagen, polyorthoesters, polyethylene glycol-coated liposomes, hyaluronic acid and/or polylactic acid. Such formulations can be prepared using standard techniques, or obtained commercially.

In some embodiments, the “pharmaceutically acceptable carrier” or “pharmaceutically acceptable excipient” includes any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs disclosed herein, can also be incorporated into the described compositions and methods.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

In some embodiments, methods disclosed herein further include administering a pharmaceutical composition comprising the gRNA to a subject. In some embodiments, the pharmaceutical composition is administered in a therapeutically effective amount to the subject.

In some embodiments, the “effective amount” or “therapeutically effective amount” refers to that amount of a compound or combination of compounds as described herein that is sufficient to effect the intended application including, but not limited to, disease treatment. In some embodiments, a therapeutically effective amount varies depending upon the intended application (in vitro or in vivo), or the subject and disease condition being treated (e.g., the weight, age and gender of the subject), the severity of the disease condition, the manner of administration, etc., which can readily be determined by one of ordinary skill in the art. The term also applies to a dose that will induce a particular response in target cells or to a target nucleic acid. In some embodiments, the specific dose varies depending on the particular compounds chosen, the dosing regimen to be followed, whether the compound is administered in combination with other compounds, timing of administration, the tissue to which it is administered, and the physical delivery system in which the compound is carried.

Still another aspect of the present disclosure provides a system comprising: a processor; and a memory storing instructions, when executed by the processor, cause the processor to perform steps comprising any of the methods and/or embodiments disclosed herein. Yet another aspect of the present disclosure provides a non-transitory computer-readable medium storing computer code comprising instructions, when executed by one or more processors, causing the processors to perform any of the methods and/or embodiments disclosed herein.

### 6.7. Examples

The following examples are illustrative of the disclosure and should not be construed as limiting in any way the general nature of the disclosure of the description throughout this specification.

### Example 1—Pretrained Model with Transformer Architecture Enhances Prediction of Editing Results

Prediction of editing results was obtained across target nucleotide positions in a target RNA using transformer-based models coupled with pretraining and further retrained using a) fine-tuning, b) linear probing, or c) linear probing and fine-tuning, in accordance with an embodiment of the present disclosure.

A model in accordance with FIGS. 3A-B was obtained. The model 130 included a first encoder block, a second encoder block, and a decoder block. Each of the first encoder block and the second encoder block was a component model pretrained on 23 million unannotated non-coding RNA sequences across 800,000 species.

The model was retrained on a training dataset including a plurality of 150,000 training samples, where each respective training sample in the plurality of training samples included training information comprising: (i) a corresponding training nucleic acid sequence for a guide RNA (gRNA)-target RNA scaffold formed between the gRNA and the target RNA when the gRNA hybridizes to the target RNA, and (ii) a corresponding training set of one or more metrics for an efficiency or specificity of deamination of a target nucleotide position in the target RNA by an Adenosine Deaminase Acting on RNA (ADAR) protein when facilitated by hybridization of the gRNA to the target RNA.

During retraining, the first encoder block received as input a first portion of the nucleic acid sequence for the gRNA-target RNA scaffold that corresponded to a sequence of the gRNA, or a representation thereof, and (ii) generated, as output, a representation of the first portion of the nucleic acid sequence. The second encoder block received as input a second portion of the nucleic acid sequence for the gRNA-target RNA scaffold that corresponded to a sequence of the target RNA, or a representation thereof, and (ii) generated, as output, a representation of the second portion of the nucleic acid sequence. The decoder block included a first portion and a second portion, where the first portion included a first attention mechanism that received, as input, the output from the first encoder block and a second attention mechanism that received, as input, the output from the second encoder block. The model then generated, as output from the decoder, a predicted set of one or more metrics for the efficiency or specificity of deamination of the target nucleotide position in the target RNA by the ADAR protein when facilitated by hybridization of the gRNA to the target RNA.

Moreover, the model was retrained using one of three approaches: fine-tuning, in which all parameters across all layers of the model were adjusted; linear probing with parameter freezing, in which parameters within lower layers were frozen and a subset of parameters within a task-specific head were adjusted; and a combination of linear probing with parameter freezing and fine-tuning, in which the linear probing approach was followed by a fine-tuning approach.

Each approach of the retrained model was validated on a validation subset of the nucleic acid sequences held out from the training dataset. Retraining approaches were validated using i) an in-distribution approach, where the held-out validation subsets had the same distribution as the training subset, or ii) an out-of-distribution approach, where the held-out validation subset had a different distribution from the training subset across one or more characteristics. In general, the model made more accurate predictions when validated using in-distribution approaches. In addition, a combined retraining approach including a combination of linear probing with parameter freezing followed by fine-tuning resulted in more accurate model predictions when validated both in-distribution and out-of-distribution.

### Example 2—Fine-Tuning Pretrained Models with Transformer Architecture Improves Performance on Editing Outcome Prediction Compared with Models Trained from Scratch

Prediction of editing outcomes was obtained using transformer-based models including different encoder blocks in an encoder-decoder architecture. Editing outcomes were compared between a convolutional neural network (CNN) encoder trained from scratch on a training dataset, a transformer encoder trained from scratch on the training dataset, and a pretrained encoder fine-tuned on the training dataset, in accordance with an embodiment of the present disclosure.

A first model including pretrained encoder blocks was obtained, in accordance with FIGS. 3A-B and Example 1. A second model including untrained CNN encoder blocks was also obtained. Finally, a third model including untrained transformer-based encoders was obtained.

Each of the three models was then trained (e.g., where the first model including pretrained encoder blocks was fine-tuned, and where the second and third models were trained from scratch) on a training dataset including a plurality of training samples (ML1 PolyTarget library). Each respective training sample in the plurality of training samples included training information comprising: (i) a corresponding training nucleic acid sequence for a guide RNA (gRNA)-target RNA scaffold formed between the gRNA and the target RNA when the gRNA hybridizes to the target RNA, and (ii) a corresponding training set of full editing outcomes (e.g., editing metrics) across a plurality of target nucleotide positions in the target RNA by an Adenosine Deaminase Acting on RNA (ADAR) protein when facilitated by hybridization of the gRNA to the target RNA. In particular, each model was trained (or fine-tuned) to predict full editing outcomes for target-guide pairs, regardless of target RNA length or format.

Each model was validated on a validation subset including unseen targets. The first model including the pretrained encoder blocks, retrained on the task-specific training dataset, outperformed both the second and third models trained from scratch (including the CNN and the transformer encoder blocks, respectively). Furthermore, while the CNN-based model easily overfitted the data, the transformer-based models (including the first and the third models) could be trained extensively without overfitting.

These data illustrate that fine-tuning pretrained models with a transformer architecture improves prediction of editing outcome, compared with models trained from scratch.

### Example 3—Training Transformer Models with Combined Sparse and Broad Dataset and Deep and Narrow Dataset Improves Performance on Editing Outcome Prediction Compared with Models Trained on Sparse and Broad Dataset

Prediction of editing outcomes comparing training datasets was obtained using transformer-based models as described above in Examples 1 and 2. The predictive output of models were compared between an XGBoost model trained on a sparse and broad training dataset for a plurality of target RNAs (XGBoost PolyTarget), a transformer model as described in Examples 1 and 2 trained on a sparse and broad training dataset for a plurality of target RNAs (Transformers PolyTarget), an XGBoost model trained on both a sparse and broad training dataset for a plurality of target RNAs and one or more deep and narrow training datasets for several single target RNAs (XGBoost PolyTarget+Additional Single Targets), and a transformer model as described in Examples 1 and 2 trained on both a sparse and broad training dataset for a plurality of target RNAs and one or more deep and narrow training datasets for several single target RNAs (Transformers PolyTarget+Additional Single Targets).

Accordingly, a sparse and broad training approach optionally with a deep and narrow training approach, were used in training models. For each model in the PolyTarget+Additional Single Target approach, the plurality of training samples included a first subset of training samples and a second subset of training samples, where each respective training sample in the first subset of training samples corresponded to a first subset of target RNA (PolyTarget) in a plurality of target RNA, each respective training sample in the second subset of training samples corresponded to a second subset of target RNA in a plurality of target RNA (Additional Single Targets), and the training included: training the model using the first subset of training samples, and training the model using the second subset of training samples. The first subset of target RNA included a greater number of target RNA than the second subset of target RNA, where the first subset of training samples was a sparse and broad training set, and the second subset of training samples was a deep and narrow training set. For each model in the PolyTarget only approach, the training was performed using the first subset of training samples only. The PolyTarget training dataset included 5,643 clinically relevant adenosine targets corresponding to approximately 2000 genes. The PolyTarget training dataset further included over 50,000 gRNAs corresponding to the 5,643 target RNAs.

Advantageously, as illustrated FIG. 8, the trained transformer models described with reference to Examples 1 and 2, when trained on a combined sparse and broad dataset and deep and narrow dataset, outperformed (indicated by arrow) the transformer model trained only on sparse and broad data, as well as the XGBoost models regardless of whether they were trained on sparse and broad data only or combined sparse and broad with deep and narrow data. FIG. 8 thus illustrates that, in some implementations, a trained model as disclosed herein, trained using both a sparse and broad approach combined with a deep and narrow approach improves the predictive output of a model compared to models that are trained only on a sparse and broad training dataset.

### Example 4—Training Transformer Models with Base-Pairing Probability Matrix Improves Performance on Editing Outcome Prediction

Prediction of editing outcomes comparing training on base-pairing probability matrix was obtained using transformer-based models as described above in Examples 1 and 2.

The predictive output of models were compared between an XGBoost model trained on a sparse and broad training dataset for a plurality of target RNAs (XGBoost PolyTarget), a transformer model as described in Examples 1 and 2 trained on a sparse and broad training dataset for a plurality of target RNAs (Transformers PolyTarget), an XGBoost model trained on both a sparse and broad training dataset for a plurality of target RNAs and one or more deep and narrow training datasets for several single target RNAs (XGBoost PolyTarget+Additional Single Targets), a transformer model as described in Examples 1 and 2 trained on both a sparse and broad training dataset for a plurality of target RNAs and one or more deep and narrow training datasets for several single target RNAs (Transformers PolyTarget+Additional Single Targets), and a transformer model as described above trained on the combined training datasets as well as on structural information provided in the form of base-pairing probability matrices (Transformers w/ BPM).

The model trained with the BPM received, as input, a nucleic acid sequence of a gRNA-target RNA scaffold and structural information including the BPM. The structural information included, for each respective position in a plurality of positions in the nucleic acid sequence of the gRNA-target RNA scaffold, a respective probability that a nucleotide base at the respective position forms a base-pair interaction with a nucleotide base at every other position in the plurality of positions other than the respective position, as a base-pairing probability matrix (BPM or BPPM).

Advantageously, as illustrated in FIG. 9, the model trained using structural information outperformed all other models not trained using structural information (indicated by arrow). FIG. 9 thus illustrates that, in some implementations, a trained model as disclosed herein, trained using both a sparse and broad approach combined with a deep and narrow approach as well as structural information (e.g., BPM) improves the predictive output of the model compared to models that are not trained on structural information.

### Example 5—Transformer-Based Models with Encoder-Decoder Structure Predict Editing Outcomes at Single Base-Pair Resolution

Models in accordance with FIG. 11 were obtained. The models included a first portion comprising an encoder block 134 and a second portion comprising a decoder block 136. The first portion comprising the encoder block 134 included one or more encoders (e.g., 12) each comprising an attention mechanism 138-1 (e.g., multi-head attention) that attended to a representation (e.g., an embedding 1102) of the nucleotide sequence of the gRNA 166, a representation (e.g., an embedding 1102) of the nucleotide sequence of the target RNA 168, and the structural information 169 about the target-guide scaffold to generate one or more embeddings 142. The second portion comprising the decoder block 136 included a corresponding a first sub-portion 144 and a second sub-portion 146, where the first sub-portion 144 included a first attention mechanism 138-3-1 that received, as input 148-1, the embedding of the nucleotide sequence of the gRNA 142-1, and a second attention mechanism 138-3-2 that received, as input, the embedding of the nucleotide sequence for the target RNA 142-2 comprising the one or more target nucleotide positions.

The models were trained with approximately 1 million in-house high-throughput nucleotide sequence data samples using a sequence-to-sequence modeling approach. Generally, sequence-to-sequence modeling approaches utilize an encoder-decoder structure and are designed to perform tasks in which one data sequence is transformed into another. Training data was subject to quality control to remove samples with low read counts and mis-folded structures. This training objective allowed the model to regress empirical Bayes calibrated editing outcomes for all adenosines in a target sequence based on a pair of target and guide sequences as input.

Four training datasets were obtained. Training dataset “ML1” included target-guide scaffold sequences for 5643 target RNAs, with 10 gRNA designs for each of the 5643 target RNAs. Training dataset “MAPT-GRN” included target-guide scaffold sequences for 5 target RNAs, including 20000 gRNA designs per target RNA. Training dataset “ML2” included target-guide scaffold sequences for 21 target RNAs overlapping with both the ML1 and MAPT-GRN training datasets, including 450 gRNA designs per target RNA. Training dataset “ML3” included target-guide scaffold sequences for the 21 target RNAs from the ML2 training dataset, including 1300 gRNA designs per target RNA. Accordingly, across the four training datasets, approximately 200,000 gRNA designs were obtained covering a total of 5648 mRNA targets, for each of the deamination enzymes ADAR1 and ADAR2.

21 target RNAs and corresponding gRNA designs unseen in any of the ML1, ML2, ML3, or MAPT-GRN training datasets were held out for testing and validation as testing datasets ML2 and ML3, including 450 gRNA designs per mRNA target and 1300 gRNA designs per mRNA target, respectively.

Models were trained on the training datasets with and without structural information in the form of base-pairing probability matrices. For each model, the encoder block (e.g., embedding model) was trained on the task of predicting a set of metrics for an efficiency or specificity of deamination of one or more target nucleotide positions in a target RNA, responsive to inputting a target-guide scaffold sequence.

Model HelixV1 was trained on the MAPT-GRN training dataset for the ADAR1 condition only, and evaluated on ML1 and MAPT-GRN validation datasets. For each respective target RNA in the training dataset, the model received, as input, a nucleotide sequence of the gRNA and a nucleotide sequence of the target RNA comprising the one or more target nucleotide positions.

Model HelixV2 was trained on the ML2 and MAPT-GRN training datasets for the ADAR1 and ADAR2 conditions, and evaluated on ML1, ML2, and MAPT-GRN validation datasets. For each respective target RNA in each training dataset, the model received, as input, a nucleotide sequence of the gRNA, a nucleotide sequence of the target RNA comprising the one or more target nucleotide positions, and structural information about the target-guide scaffold.

Advantageously, the transformer-based models were capable of handling inputs with variable lengths, and as such were generalizable to different micro-footprints or macro-footprints for in-cell experiments. Additionally, the transformer-based models were less prone to overfitting, allowing for easy integration of historical or future datasets to further enhance the model performance. Advantageously, the embeddings generated by the encoder blocks further included meaningful information on RNA biology, enabling other tasks through transfer learning.

### Example 6—Transfer Learning Using Embeddings from Transformer-Based Models Improves Generative gRNA Design Over Bit Diffusion Model Generation

Models HelixV1 and HelixV2 were obtained and trained as described in Example 5, above. A generative model was obtained as follows:

The encoder block (e.g., the embedding model) for each of HelixV1 and HelixV2 was used to generate a respective embedding dataset comprising a corresponding plurality of embeddings, responsive to inputting target-guide sequences from the ML1 training dataset to both models. Each of the HelixV1 and HelixV2 embedding datasets were then used to generate a respective noised embedding training dataset by adding noise (e.g., dropout, stochastic depth, and/or data augmentation) to each embedding in the corresponding plurality of embeddings. Each of the HelixV1 and HelixV2 noised embedding training datasets were further used to train the generative model to generate gRNA designs by de-noising the noised embeddings. In particular, responsive to inputting each respective noised embedding in a respective training dataset, the generative model was trained on the task of de-noising the guide RNA embedding depending on the target RNA embedding and other conditions by predicting the noise and back-propagating on the error.

The trained generative model was then evaluated using noised sequences from the MAPT-GRN (noise: 0.95, 0.93), ML2 (noise: 0.80), and ML3 (noise: 0.80) datasets. The generated gRNA designs were evaluated based on sequence length distribution, mismatch pairs distribution (e.g., pairs that are not A-T or G-C), plagiarism to the training dataset, minimum free energy (MFE) distribution, and prediction of one or more metrics for an efficiency or specificity of deamination of the one or more target nucleotide positions in the target RNA by a deamination enzyme when facilitated by hybridization of the gRNA to the target RNA. The prediction of the deamination efficiency or specificity metrics was obtained using an XGBoost predictive model trained on train-test splits of the ML1, MAPT-GRN, or ML2 datasets and fit on the ML2 and ML3 training datasets to improve predictions.

Predictions obtained from the XGBoost model were evaluated using score estimates, score noise, and score bias to compare the performance of gRNA designs generated by the different generative models trained on different embedding datasets (e.g., HelixV1, HelixV2). The score estimate refers to the Spearman correlation coefficient between a target (e.g., desired) editing or specificity on the target RNA versus the predicted editing or specificity of the generated gRNA design. Higher values indicate higher correlation between the predicted editing or specificity and the target (e.g., desired) editing or specificity and indicates the ability of the XGBoost model to predict editing and specificity metrics. The score noise is a single value metric of the XGBoost efficiency itself and does not depend on the generative model. Score noise refers to the Spearman correlation coefficient between the target (e.g., desired) editing or specificity on the target RNA versus the predicted editing or specificity of an “optimal” gRNA design for the target RNA. Higher score noise values indicate lower noise and higher confidence in XGBoost predictions. The score bias measures the error variance in the XGBoost prediction of the generated guide due to its optimization bias. Score bias refers to the Spearman correlation coefficient between the predicted editing or specificity of an “optimal” gRNA design for the target RNA versus the predicted editing or specificity of the generated gRNA design.

A bit diffusion model was trained on the task of generating gRNA designs on noised sequences from the ML1 training dataset and evaluated using noised sequences from the MAPT-GRN (noise: 0.95, 0.93), ML2 (noise: 0.80), and ML3 (noise: 0.80) datasets. The gRNA designs generated by the bit diffusion model were evaluated using the XGBoost predictive model to obtain predictions of deamination efficiency or specificity metrics.

Performance of gRNA designs generated by the HelixV1-trained generative model (e.g., embeddings from HelixV1, not trained on structural features), the HelixV2-trained generative model (e.g., embeddings from HelixV2, trained on structural features), and the bit diffusion model (e.g., not trained on embeddings) were then compared using XGBoost score estimates for ADAR1 editing (e.g., efficiency) and specificity, as illustrated in Table 2.

TABLE 2
Comparison of gRNA Designs Generated from Transfer Learning.

| | | | Baseline |
|---|---|---|---|
| | HelixV1 | HelixV2 | Bit |
| Dataset \| Condition \|XGBoost Noise | Score | Score | Diffusion |
| MAPT-GRN Editing (Noise 0.95) | 0.85 | 0.77 | 0.80 |
| MAPT-GRN Specificity (Noise 0.93) | 0.81 | 0.66 | 0.76 |
| ML2 & ML3 Editing (Noise 0.80) | 0.77 | 0.70 | 0.70 |
| ML2 & ML3 Specificity (Noise 0.80) | 0.75 | 0.77 | 0.74 |

The gRNA designs obtained from generative models trained on noised embeddings outperformed the gRNA designs obtained from a bit diffusion model that was not trained on embeddings. Furthermore, embeddings obtained from models trained on structural information (e.g., base-pairing probability matrices) resulted in gRNA designs having generally comparable performance relative to models not trained on structural information, with increased specificity of editing observed when evaluating on the ML2 and ML3 datasets.

### Example 7—Augmentation of Training Datasets Using Noisy Student Training Improves Deamination Efficiency and Specificity of gRNA Designs Obtained from Bit Diffusion Models

Model HelixV2 was obtained and trained as described in Examples 5 and 6, above.

The encoder block (e.g., the embedding model) for HelixV2 was used to generate a respective embedding dataset comprising a corresponding plurality of embeddings, responsive to inputting target-guide sequences from the ML1 training dataset to the model. The HelixV2 embedding dataset was augmented by a noisy student training process comprising: adding noise (e.g., dropout, stochastic depth, and/or data augmentation) to one or more embeddings in the embeddings dataset, thereby obtaining one or more noised embeddings; and adding the one or more noised embeddings to the embeddings dataset. 5 augmented datasets were obtained after augmenting with 50 noisy generated gRNAs (NS-50), 100 noisy generated gRNAs (NS-100), 200 noisy generated gRNAs (NS-200), 400 noisy generated gRNAs (NS-400), and 600 noisy generated gRNAs (NS-600) (data not shown). Each augmented embedding dataset was further used to generate a respective noised, augmented embedding training dataset by adding noise (e.g., dropout, stochastic depth, and/or data augmentation) to each embedding in the corresponding plurality of embeddings.

Each noised, augmented embedding training dataset was further used to train a bit diffusion model on the task of generating gRNA designs from noised sequences. In particular, responsive to inputting each respective noised embedding in the noised embedding training dataset, the bit diffusion model was trained on the task of de-noising the guide RNA embedding depending on the target RNA embedding and other conditions by predicting the noise and back-propagating on the error. gRNA designs generated from an ML1 validation set were evaluated using the XGBoost predictive model as described in Example 6. Bit diffusion models were further trained on MAPT-GRN and ML2 datasets and gRNA designs generated from these models were compared against the gRNA designs generated using noisy student training. Table 3 illustrates the comparison between XGBoost score estimates for ADAR1 editing (e.g., efficiency) and specificity.

TABLE 3
Score Estimates for gRNA Designs Obtained from Noisy
Student-Augmented Versus Non-Augmented Datasets.

| Augmentation (X) | Metric | Epoch | ML1* | MAPT-GRN | ML2 |
|---|---|---|---|---|---|
| 50* | Editing | 9 | 0.55 | 0.73 | 0.45 |
| | | 4999 | 0.61 | 0.79 | 0.45 |
| | Specificity | 9 | 0.81 | 0.41 | 0.58 |
| | | 4999 | 0.84 | 0.59 | 0.68 |
| 100* | Editing | 9 | N/A | 0.45 | 0.24 |
| | | 2499 | 0.62 | 0.80 | 0.45 |
| | Specificity | 9 | N/A | 0.12 | 0.34 |
| | | 2499 | 0.86 | 0.40 | 0.67 |
| 200* | Editing | 9 | 0.36 | 0.49 | 0.27 |
| | | 4999 | 0.66 | 0.80 | 0.49 |
| | Specificity | 9 | 0.73 | 0.19 | 0.46 |
| | | 4999 | 0.86 | 0.47 | 0.67 |
| 400* | Editing | 9 | 0.47 | 0.54 | 0.29 |
| | | 4999 | 0.73 | 0.81 | 0.51 |
| | Specificity | 9 | 0.75 | 0.25 | 0.50 |
| | | 4999 | 0.86 | 0.42 | 0.67 |

*The MAPT-GRN and ML2 datasets were not augmented by noisy student training.

Table 3 shows the XGBoost estimated scores for editing (e.g., efficiency) and specificity by ADAR1. The estimated score indicates the Spearmnan correlation coefficient (ρ) of the target (e.g., desired) editing or specificity of the generated guide with the predicted editing or specificity of the generated guide, as made by the trained XGBoost model (e.g., how well the generated gRNA designs perform relative to target performance).

Bit diffusion models trained on the MAPT-GRN training datasets generated higher performing guides compared to those trained on ML2 training datasets, as expected due to the higher the number of sequences per target available in these training datasets. Nevertheless, the noisy student-augmented ML1 datasets gave rise to gRNA designs that exhibited higher specificity relative even to the MAPT-GRN-derived guides, indicating that augmentation of training datasets using noisy student training can improve the design and performance of guide RNAs for target RNAs.

### 6.8. Example Clauses

The following clauses describe specific embodiments of the disclosure.
Clause 1. A method for predicting a deamination efficiency or specificity at one or more target nucleotide positions of a target RNA, comprising: at a computer system comprising at least one processor and a memory storing at least one program for execution by the at least one processor: inputting information about a target-guide scaffold formed between a guide RNA (gRNA) and the target RNA when the gRNA hybridizes to the target RNA into a model to receive as output from the model a predicted set of one or more metrics for an efficiency or specificity of deamination of the one or more target nucleotide positions in the target RNA by a deamination enzyme when facilitated by hybridization of the gRNA to the target RNA, wherein: the information comprises a nucleotide sequence of the gRNA, a nucleotide sequence of the target RNA comprising the one or more target nucleotide positions, and structural information about the target-guide scaffold, and the model comprises: a first portion comprising one or more encoder blocks that attend to a representation of the nucleotide sequence of the gRNA, a representation of the nucleotide sequence of the target RNA, and a representation of the structural information about the target-guide scaffold to generate one or more embeddings; and a second portion comprising one or more decoder blocks that attend to the one or more embeddings to generate the predicted set of one or more metrics.
Clause 2. The method of clause 1, wherein the structural information comprises a corresponding plurality of structural features of the target-guide scaffold formed between the gRNA and the target RNA when the gRNA hybridizes to the target RNA.
Clause 3. The method of clause 2, wherein the corresponding plurality of structural features comprises one or more of a micro-footprint and a macro-footprint.
Clause 4. The method of any one of clauses 1-3, wherein the structural information comprises a plurality of secondary structural features.
Clause 5. The method of any one of clauses 1-4, wherein the structural information comprises tertiary structural features.
Clause 6. The method of clause 4 or 5, wherein the structural information comprises, for each respective position in a plurality of positions in a nucleic acid sequence for the target-guide scaffold, a respective probability that a nucleotide base at the respective position will form a base-pair interaction with a nucleotide base at every other position in the plurality of positions other than the respective position.
Clause 7. The method of clause 6, wherein the structural information comprises a base-pairing probability matrix.
Clause 8. The method of clause 7, further comprising: repeating the inputting, for each respective gRNA-target RNA scaffold in a plurality of gRNA-target RNA scaffolds, thereby receiving, for each respective gRNA-target RNA scaffold in the plurality of gRNA-target RNA scaffolds, a corresponding predicted set of one or more metrics for the efficiency or specificity of deamination of the one or more target nucleotide positions in the target RNA by a deamination enzyme when facilitated by hybridization of the gRNA to the target RNA.
Clause 9. The method of any one of clauses 1-8, wherein the model further generates an estimation of a minimum free energy (MFE) for the gRNA.
Clause 10. The method of any one of clauses 1-9, wherein the model further generates an estimation of a minimum free energy (MFE) for the target-guide scaffold formed between the guide RNA (gRNA) and the target RNA.
Clause 11. The method of any one of clauses 1-10, wherein the output from the model further comprises a metric for an efficiency or specificity of deamination of one or more nucleotide positions, other than the one or more target nucleotide positions, in the target RNA by the deamination enzyme when facilitated by hybridization of the gRNA to the target RNA.
Clause 12. The method of any one of clauses 1-11, wherein the deamination enzyme is an Adenosine Deaminase Acting on RNA (ADAR protein).
Clause 13. The method of clause 12, wherein the ADAR protein is human ADAR1 or human ADAR2.
Clause 14. The method of any one of clauses 1-13, wherein the predicted set of one or more metrics for the efficiency or specificity of deamination of the one or more target nucleotide positions by the deamination enzyme comprises a metric for the efficiency of deamination of the one or more target nucleotide positions by the deamination enzyme.
Clause 15. The method of any one of clauses 1-14, wherein the predicted set of one or more metrics for the efficiency or specificity of deamination of the one or more target nucleotide positions by the deamination enzyme comprises a metric for the specificity of deamination of the one or more target nucleotide positions relative to one or more nucleotide positions, other than the one or more target nucleotide positions, in the target RNA by the deamination enzyme.
Clause 16. The method of clause 15, wherein a metric for the specificity of deamination of the one or more target nucleotide positions relative to one or more nucleotide positions, other than the target nucleotide position, in the target RNA by the deamination enzyme is selected from the group consisting of: (i) a comparison of (a) a prevalence of deamination of the one or more target nucleotide positions in a plurality of instances of the target RNA and (b) a prevalence of deamination of at least one nucleotide position, other than the one or more target nucleotide positions, in a respective instance of the target RNA in a plurality of instances of the target RNA, (ii) a prevalence of deamination of the one or more target nucleotide positions, without coincident deamination of one or more nucleotide positions other than the one or more target nucleotide positions, in a respective instance of the target RNA in a plurality of instances of the target RNA, and (iii) a prevalence of deamination of at least one nucleotide position, other than the one or more target nucleotide positions, in a respective instance of the target RNA in a plurality of instances of the target RNA.
Clause 17. The method of clause 16, wherein, at the at least one nucleotide position, other than the target nucleotide position, in the target RNA, deamination results in a non-synonymous codon edit.
Clause 18. The method of any one of clauses 1-17, wherein a respective metric in the predicted set of one or more metrics for the efficiency or specificity of deamination of the one or more target nucleotide positions by the deamination enzyme is normalized by a metric for an efficiency or specificity of deamination of one or more nucleotide positions, other than the one or more target nucleotide positions, in the target RNA by the deamination enzyme.
Clause 19. The method of any one of clauses 1-18, wherein the predicted set of one or more metrics further includes an efficiency or specificity of deamination of each target nucleotide position, in a plurality of target nucleotide positions, in the target RNA by the deamination enzyme when facilitated by hybridization of the gRNA to the target RNA.
Clause 20. The method of any one of clauses 1-19, wherein the predicted set of one or more metrics further includes an efficiency or specificity of deamination of each nucleotide position, in a plurality of nucleotide positions, in the target RNA by the deamination enzyme when facilitated by hybridization of the gRNA to the target RNA.
Clause 21. The method of any one of clauses 1-20, wherein the predicted set of one or more metrics further includes an efficiency or specificity of deamination of each adenosine in the target RNA by the deamination enzyme when facilitated by hybridization of the gRNA to the target RNA.
Clause 22. The method of any one of clauses 1-21, wherein the gRNA comprises at least 25 nucleotides.
Clause 23. The method of any one of clauses 1-22, wherein the information further comprises a nucleic acid sequence for the target RNA comprising a first sub-sequence flanking a 5′ side of a target nucleotide position in the target RNA and a second sub-sequence flanking a 3′ side of the target nucleotide position in the target RNA.
Clause 24. The method of clause 8, wherein the plurality of gRNA-target RNA scaffolds comprises at least 1000, at least 10,000, at least 100,000, at least 500,000, at least 1×10⁶, at least 1×10⁷, or at least 1×10⁸ gRNA-target RNA scaffolds.
Clause 25. The method of any one of clauses 1-24, wherein the model comprises a language model, a transformer model, a large language model (LLM), an encoder, a decoder, an encoder-decoder hybrid model, a generative pre-trained transformer (GPT) model, a Bidirectional Encoder Representations from Transformers (BERT) model, or a multiple sequence alignment (MSA) transformer model.
Clause 26. The method of any one of clauses 1-25, wherein the model comprises RNA-FM, RNA-MSM, Atom-1, or BigRNA.
Clause 27. The method of any one of clauses 1-26, wherein one or more of the first portion of the model and the second portion of the model comprises an attention mechanism.
Clause 28. The method of clause 27, wherein the attention mechanism is selected from the group consisting of dot product attention, query-key-value attention, Luong attention, and Bahdanau attention.
Clause 29. The method of clause 27 or 28, wherein the model further comprises an output layer and each of the first portion and the second portion comprises: an input layer, and a plurality of hidden layers comprising (i) a first sub-portion that comprises the attention mechanism, and (ii) a second sub-portion.
Clause 30. The method of clause 29, wherein, for each of the first portion and the second portion, the corresponding second sub-portion comprises a neural network, a support vector machine, a Naive Bayes model, a nearest neighbor model, a boosted trees model, a random forest model, a decision tree, or a clustering model.
Clause 31. The method of clause 29 or 30, wherein the second sub-portion of the model comprises an extreme gradient boost (XGBoost) model.
Clause 32. The method of clause 29 or 30, wherein the second sub-portion of the model comprises a convolutional or graph-based neural network.
Clause 33. The method of any one of clauses 29-32, wherein the model receives, responsive to inputting the information about the target-guide scaffold: at the input layer of the first portion: (i) the nucleotide sequence of the gRNA, or a representation thereof, (ii) the nucleotide sequence of the target RNA comprising the one or more target nucleotide positions, or a representation thereof, and (iii) the structural information about the target-guide scaffold.
Clause 34. The method of clause 33, wherein the model receives, at the first sub-portion that comprises the attention mechanism: (i) the nucleotide sequence of the gRNA, or the representation thereof, (ii) the nucleotide sequence of the target RNA comprising the one or more target nucleotide positions, or the representation thereof, and (iii) the structural information about the target-guide scaffold.
Clause 35. The method of clause 33 or 34, further comprising embedding a nucleic acid sequence for the target-guide scaffold prior to inputting to the first portion.
Clause 36. The method of any one of clauses 1-35, wherein the one or more embeddings comprises one or more of: a first embedding for the nucleotide sequence of the gRNA, and a second embedding for the nucleotide sequence of the target RNA comprising the one or more target nucleotide positions.
Clause 37. The method of clause 36, wherein the first portion generates, as output, (i) an embedding for the nucleotide sequence of the gRNA, and (ii) an embedding for the nucleotide sequence of the target RNA comprising the one or more target nucleotide positions.
Clause 38. The method of clause 37, wherein the first portion generates the first embedding for the nucleotide sequence of the gRNA by considering one or more of: (i) the nucleotide sequence of the gRNA, (ii) the nucleotide sequence of the target RNA, and (iii) the structural information about the target-guide scaffold.
Clause 39. The method of clause 37, wherein the first portion generates the first embedding for the nucleotide sequence of the gRNA by considering only the nucleotide sequence of the target RNA.
Clause 40. The method of clause 37, wherein the first portion generates the first embedding for the nucleotide sequence of the gRNA by considering the nucleotide sequence of the target RNA and a nucleotide sequence having perfect complementarity to the nucleotide sequence of the target RNA.
Clause 41. The method of clause 37, wherein the first portion generates the first embedding for the nucleotide sequence of the gRNA by considering the nucleotide sequence of the target RNA, a nucleotide sequence having perfect complementarity to the nucleotide sequence of the target RNA, and structural information about a target-sequence scaffold formed between the target RNA and the nucleotide sequence having perfect complementarity to the nucleotide sequence of the target RNA.
Clause 42. The method of any one of clauses 37-41, wherein the first portion generates the second embedding for the nucleotide sequence of the target RNA by considering one or more of: (i) the nucleotide sequence of the gRNA, (ii) the nucleotide sequence of the target RNA, and (iii) the structural information about the target-guide scaffold.
Clause 43. The method of any one of clauses 37-41, wherein the first portion generates the second embedding for the nucleotide sequence of the target RNA by considering: only the nucleotide sequence of the target RNA, the nucleotide sequence of the target RNA and a nucleotide sequence having perfect complementarity to the nucleotide sequence of the target RNA, or the nucleotide sequence of the target RNA, the nucleotide sequence having perfect complementarity to the nucleotide sequence of the target RNA, and structural information about a target-sequence scaffold formed between the target RNA and the nucleotide sequence having perfect complementarity to the nucleotide sequence of the target RNA.
Clause 44. The method of any one of clauses 37-43, wherein information considered by the first portion of the model to generate the first embedding is the same or different from information considered by the first portion of the model to generate the second embedding.
Clause 45. The method of any one of clauses 36-44, wherein the second portion comprises (i) a first attention mechanism that receives, as input, an embedding for the nucleotide sequence of the gRNA, or the representation thereof, and (ii) a second attention mechanism that receives, as input, an embedding for the nucleotide sequence of the target RNA comprising the one or more target nucleotide positions, or the representation thereof.
Clause 46. The method of any one of clauses 1-45, wherein the one or more embeddings comprises an embedding for a nucleic acid sequence for the target-guide scaffold, wherein the nucleic acid sequence of the target-guide scaffold comprises the nucleotide sequence of the gRNA and the nucleotide sequence of the target RNA comprising the one or more target nucleotide positions.
Clause 47. The method of clause 46, wherein the first portion generates the embedding for the nucleic acid sequence for the target-guide scaffold by considering one or more of: (i) the nucleotide sequence of the gRNA, (ii) the nucleotide sequence of the target RNA, and (iii) the structural information about the target-guide scaffold.
Clause 48. The method of clause 46, wherein the first portion generates the embedding for the nucleic acid sequence for the target-guide scaffold by considering: only the nucleotide sequence of the target RNA, the nucleotide sequence of the target RNA and a nucleotide sequence having perfect complementarity to the nucleotide sequence of the target RNA, or the nucleotide sequence of the target RNA, the nucleotide sequence having perfect complementarity to the nucleotide sequence of the target RNA, and structural information about a target-sequence scaffold formed between the target RNA and the nucleotide sequence having perfect complementarity to the nucleotide sequence of the target RNA.
Clause 49. The method of any one of clauses 45-48, wherein the second portion comprises an attention mechanism that receives, as input, the embedding for a nucleotide sequence of the target-guide scaffold.
Clause 50. The method of any one of clauses 1-49, further comprising using the model to obtain an embeddings dataset, comprising: determining an initial dataset comprising, for each respective sample in a plurality of samples: a corresponding nucleic acid sequence for a gRNA-target RNA scaffold formed between the gRNA and the target RNA when the gRNA hybridizes to the target RNA, and corresponding structural information about the target-guide scaffold; inputting, to the first portion of the model, for each respective sample in the plurality of samples: (i) a nucleotide sequence of the gRNA, or a representation thereof, (ii) a nucleotide sequence of the target RNA comprising the one or more target nucleotide positions, or a representation thereof, and (iii) the corresponding structural information about the target-guide scaffold; and obtaining, as output from the first portion of the model, for each respective sample in the plurality of samples, a respective embedding comprising: an embedding for the nucleotide sequence of the gRNA, and an embedding for the nucleotide sequence of the target RNA comprising the one or more target nucleotide positions.
Clause 51. The method of clause 50, further comprising obtaining an augmented embeddings dataset, comprising: adding noise to one or more embeddings in the embeddings dataset, thereby obtaining one or more noised embeddings; and adding the one or more noised embeddings to the embeddings dataset.
Clause 52. The method of clause 51, wherein the augmented embeddings dataset is obtained using noisy student training or noisy student distillation.
Clause 53. The method of clause 51 or 52, wherein the noise comprises one or more of dropout, stochastic depth, and data augmentation.
Clause 54. The method of any one of clauses 51-53, further comprising using the augmented embeddings dataset to train a generative model, wherein the generative model determines a nucleic acid sequence for a guide RNA.
Clause 55. The method of clause 54, wherein the generative model comprises a bit diffusion model.
Clause 56. The method of any one of clauses 29-32, wherein the first portion comprises an encoder block, the second portion comprises a decoder block, and the model receives, responsive to inputting information comprising a corresponding nucleic acid sequence for a gRNA-target RNA scaffold: at an input layer of the first portion, a portion of a nucleic acid sequence for the gRNA-target RNA scaffold that corresponds to the nucleic acid sequence for the gRNA, or a representation thereof, and at an input layer of the second portion, a portion of the nucleic acid sequence for the gRNA-target RNA scaffold that corresponds to the nucleic acid sequence for the target RNA, or a representation thereof.
Clause 57. The method of any one of clauses 1-56, wherein the model comprises at least 500,000 parameters, at least 1×10⁶ parameters, at least 1×10⁷ parameters, at least 1×10⁸ parameters, at least 1×10⁹ parameters, at least 1×10¹⁰ parameters, at least 1×10¹¹ parameters, or at least 2×10¹¹ parameters.
Clause 58. The method of any one of clauses 1-57, wherein the structural information is provided as input to one or both of the first portion and the second portion of the model.
Clause 59. The method of clause 58, wherein the first portion comprises an encoder block, the second portion comprises a decoder block, and the model receives, responsive to inputting information comprising a corresponding nucleic acid sequence for a gRNA-target RNA scaffold: at an input layer of the first portion, (i) a first portion of a nucleic acid sequence for the gRNA-target RNA scaffold that corresponds to the nucleic acid sequence for the gRNA, or a representation thereof, and (ii) all or a portion of the structural information comprising, for each respective position in a plurality of positions in the nucleic acid sequence for the gRNA-target RNA scaffold, a respective probability that a nucleotide base at the respective position will form a base-pair interaction with a nucleotide base at every other position in the plurality of positions other than the respective position, and at an input layer of the second portion, (i) a second portion of the nucleic acid sequence for the gRNA-target RNA scaffold that corresponds to the nucleic acid sequence for the target RNA, or a representation thereof, and (ii) all or a portion of the structural information comprising, for each respective position in a plurality of positions in the nucleic acid sequence for the gRNA-target RNA scaffold, a respective probability that a nucleotide base at the respective position will form a base-pair interaction with a nucleotide base at every other position in the plurality of positions other than the respective position.
Clause 60. The method of any one of clauses 1-59, wherein the model further comprises a third portion, wherein: the third portion comprises one or more encoder blocks, and the model receives, responsive to inputting information comprising a corresponding nucleic acid sequence for a gRNA-target RNA scaffold, at an input layer of the third portion, the structural information for the corresponding nucleic acid sequence.
Clause 61. The method of clause 60, wherein the third portion comprises an attention mechanism.
Clause 62. The method of clause 61, wherein the third portion comprises: an input layer, and a plurality of hidden layers comprising (i) a first sub-portion that comprises the attention mechanism, and (ii) a second sub-portion.
Clause 63. The method of clause 62, wherein the second sub-portion of the third portion comprises a neural network, a support vector machine, a Naive Bayes model, a nearest neighbor model, a boosted trees model, a random forest model, a decision tree, or a clustering model.
Clause 64. A method for predicting a deamination efficiency or specificity comprising: at a computer system comprising at least one processor and a memory storing at least one program for execution by the at least one processor: A) obtaining a model comprising a first encoder block, a second encoder block, and a decoder block, wherein: the decoder block comprises a first portion and a second portion, wherein the first portion comprises a first attention mechanism that receives, as input, an output from the first encoder block and a second attention mechanism that receives, as input, an output from the second encoder block; B) inputting, into the model, information comprising a nucleic acid sequence for a target-guide scaffold formed between a gRNA and the target RNA when the gRNA hybridizes to the target RNA; and C) receiving, as output from the model, a predicted set of one or more metrics for the efficiency or specificity of deamination of one or more target nucleotide positions in the target RNA by a deamination enzyme when facilitated by hybridization of the gRNA to the target RNA.
Clause 65. A method for predicting a deamination efficiency or specificity comprising: at a computer system comprising at least one processor and a memory storing at least one program for execution by the at least one processor: A) obtaining a model comprising a first encoder block, a second encoder block, and a decoder block, wherein: the first encoder block comprises a first set of parameters, in a plurality of parameters of the model, the second encoder block comprises a second set of parameters, in the plurality of parameters of the model, and the decoder block comprises a third set of parameters, in the plurality of parameters of the model; B) inputting, into the model, information comprising a nucleic acid sequence for a guide RNA (gRNA)-target RNA scaffold formed between the gRNA and the target RNA when the gRNA hybridizes to the target RNA, wherein: the first encoder block (i) receives, as input, a first portion of the nucleic acid sequence for the gRNA-target RNA scaffold that corresponds to a sequence of the gRNA, or a representation thereof, and (ii) generates, as output, a representation of the first portion of the nucleic acid sequence, the second encoder block (i) receives, as input, a second portion of the nucleic acid sequence for the gRNA-target RNA scaffold that corresponds to a sequence of the target RNA, or a representation thereof, and (ii) generates, as output, a representation of the second portion of the nucleic acid sequence, and the decoder block comprises a first portion and a second portion, wherein the first portion comprises a first attention mechanism that receives, as input, the output from the first encoder block and a second attention mechanism that receives, as input, the output from the second encoder block; and C) receiving, as output from the model, a predicted set of one or more metrics for the efficiency or specificity of deamination of one or more target nucleotide positions in the target RNA by a deamination enzyme when facilitated by hybridization of the gRNA to the target RNA.
Clause 66. The method of clause 54, wherein each of the first encoder block and the second encoder block comprises: a first portion and a second portion, wherein the first portion comprises an attention mechanism that receives, as input, the corresponding portion of the nucleic acid sequence for the gRNA-target RNA scaffold, or the representation thereof.
Clause 67. A system comprising: a processor; and a memory storing instructions, when executed by the processor, cause the processor to perform steps comprising the method of any one of clauses 1-66.
Clause 68. A non-transitory computer-readable medium storing computer code comprising instructions, when executed by one or more processors, causing the processors to perform the method of any one of clauses 1-66.

### 6.9. Additional Considerations

All references cited herein are incorporated by reference to the same extent as if each individual publication, database entry (e.g., Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The foregoing description of the embodiments has been presented for the purpose of illustration; it is not intended to be exhaustive or to limit the patent rights to the precise forms disclosed. Persons skilled in the relevant art will appreciate that many modifications and variations are possible in light of the above disclosure.

Any feature mentioned in one claim category, e.g., method, can be claimed in another claim category, e.g., computer program product, system, storage medium, as well. The dependencies or references back in the attached claims are chosen for formal reasons only. However, any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject matter will be understood to include not only the combinations of features as set out in the disclosed embodiments but also any other combination of features from different embodiments. Various features mentioned in the different embodiments can be combined with explicit mentioning of such combination or arrangement in an example embodiment or without any explicit mentioning. Furthermore, any of the embodiments and features described or depicted herein can be claimed in a separate claim and/or in any combination with any embodiment or feature described or depicted herein or with any of the features.

Some portions of this description describe the embodiments in terms of algorithms and symbolic representations of operations on information. These operations and algorithmic descriptions, while described functionally, computationally, or logically, are understood to be implemented by computer programs or equivalent electrical circuits, microcode, or the like. Furthermore, it has also proven convenient at times, to refer to these arrangements of operations as engines, without loss of generality. The described operations and their associated engines are, in some embodiments, embodied in software, firmware, hardware, or any combinations thereof.

Any of the steps, operations, or processes described herein, in some embodiments, are performed or implemented with one or more hardware or software engines, alone or in combination with other devices. In one embodiment, a software engine is implemented with a computer program product comprising a computer-readable medium containing computer program code, which can be executed by a computer processor for performing any or all of the steps, operations, or processes described. The term “steps” does not mandate or imply a particular order. For example, while this disclosure describes, in some embodiments, a process that includes multiple steps sequentially with arrows present in a flowchart, the steps in the process do not need to be performed by the specific order claimed or described in the disclosure. In some implementations, some steps are performed before others even though the other steps are claimed or described first in this disclosure. Likewise, any use of (i), (ii), (iii), etc., or (a), (b), (c), etc., in the specification or in the claims, unless specified, is used to better enumerate items or steps and also does not mandate a particular order.

Throughout this specification, plural instances may implement components, operations, or structures described as a single instance. Although individual operations of one or more methods are illustrated and described as separate operations, in some implementations one or more of the individual operations are performed concurrently, and nothing requires that the operations be performed in the order illustrated. Structures and functionality presented as separate components in example configurations are, in some embodiments, implemented as a combined structure or component. Similarly, in some embodiments, structures and functionality presented as a single component are implemented as separate components. These and other variations, modifications, additions, and improvements fall within the scope of the subject matter herein. In addition, the term “each” used in the specification and claims does not imply that every or all elements in a group need to fit the description associated with the term “each.” For example, “each member is associated with element A” does not imply that all members are associated with an element A. Instead, the term “each” only implies that a member (of some of the members), in a singular form, is associated with an element A. In claims, in some instances, the use of a singular form of a noun implies at least one element even though a plural form is not used.

Finally, the language used in the specification has been principally selected for readability and instructional purposes, rather than selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights.

Although inventions have been particularly shown and described with reference to a preferred embodiment and various alternate embodiments, it will be understood by persons skilled in the relevant art that various changes in form and details can be made therein without departing from the spirit and scope of the invention.

What is claimed is:

1. A method for predicting a deamination efficiency or specificity comprising:
at a computer system comprising at least one processor and a memory storing at least one program for execution by the at least one processor:
A) obtaining a model comprising an encoder block and a decoder block, wherein:
the encoder block comprises a first set of parameters, in a plurality of parameters of the model and the decoder block comprises a second set of parameters, in the plurality of parameters of the model;
B) inputting, into the model, (i) information comprising a nucleic acid sequence for a target-guide scaffold formed between a guide RNA (gRNA) and a target RNA when the gRNA hybridizes to the target RNA, wherein the nucleic acid sequence for the target-guide scaffold comprises a first component corresponding to the gRNA and a second component corresponding to the target RNA, and (ii) structural information for the target-guide scaffold comprising a base-pairing probability matrix, wherein:
the encoder block comprises a first attention mechanism that receives the information comprising the nucleic acid sequence for the target-guide scaffold and the structural information for the target-guide scaffold, and
the decoder block comprises a corresponding a first sub-portion and a second sub-portion, wherein the first sub-portion comprises a second attention mechanism and a third attention mechanism, wherein the decoder block receives, as input, an output generated from the encoder block; and
C) receiving, as output from the model, a predicted set of one or more metrics for the efficiency or specificity of deamination of one or more target nucleotide positions in the target RNA by a deamination enzyme when facilitated by hybridization of the gRNA to the target RNA.

2. The method of claim 1, wherein the nucleic acid sequence for the target-guide scaffold comprises all or a portion of the guide-target scaffold.

3. The method of claim 1 or 2, wherein the nucleic acid sequence for the target-guide scaffold comprises one or more macro-footprint structural features.

4. The method of claim 3, wherein the one or more macro-footprint structural features comprises one or more barbells.

5. The method of claim 3 or 4, wherein the one or more macro-footprint structural features are positioned at one or both ends of the target-guide scaffold inputted to the model.

6. The method of claim 3 or 4, wherein the one or more macro-footprint structural features are positioned at other than an end of the target-guide scaffold inputted to the model.

7. The method of any one of claims 1-6, wherein the information comprising the nucleic acid sequence for the target-guide scaffold comprises a tensor having the dimensions l×d, wherein l is a positive integer representing a number of nucleotide positions in the nucleic acid sequence for the target-guide scaffold.

8. The method of claim 7, wherein l is a positive integer from 100 to 300.

9. The method of claim 7 or 8, wherein d is a positive integer representing a number of component encoders in the encoder block.

10. The method of claim 9, wherein the encoder block comprises a plurality of component encoders, and wherein d is a positive integer from 3 to 40.

11. The method of claim 10, wherein the first attention mechanism is a multi-head attention mechanism comprising a plurality of attention heads, and wherein each component encoder in d corresponds to a respective attention head in the plurality of attention heads.

12. The method of any one of claims 1-11, further comprising, prior to inputting into the information comprising the nucleic acid sequence for the target-guide scaffold into the encoder block, embedding the nucleic acid sequence for the target-guide scaffold using linear mapping or matrix multiplication.

13. The method of claim 12, further comprising, prior to inputting the information comprising the nucleic acid sequence for the target-guide scaffold into the encoder block, encoding the nucleic acid sequence for the target-guide scaffold using positional encoding.

14. The method of any one of claims 1-13, wherein the information comprising the nucleic acid sequence and the base pairing matrix, or representations thereof, are inputted separately into the model

15. The method of any one of claims 1-14, wherein the first attention mechanism is a multi-head attention mechanism comprising a plurality of attention heads.

16. The method of claim 15, wherein the plurality of attention heads comprises at least 5, at least 10, or at least 15 attention heads.

17. The method of claim 15, wherein the plurality of attention heads consists of from 3 to 40 attention heads.

18. The method of any one of claims 1-17, wherein the base-pairing probability matrix comprises dimensions l×l×m, wherein l is a positive integer representing a number of nucleotide positions in the nucleic acid sequence for the target-guide scaffold.

19. The method of claim 18, wherein l is a positive integer from 100 to 300.

20. The method of claim 18 or 19, wherein m is positive integer representing a number of attention heads in the encoder block.

21. The method of claim 20, wherein the first attention mechanism is a multi-head attention mechanism comprising a plurality of attention heads, and wherein m is a positive integer from 3 to 40.

22. The method of claim 21, wherein the structural information comprises, for each respective attention head in the plurality of attention heads, a corresponding iteration of the base pairing probability matrix, and wherein each respective attention head in the plurality of attention heads in the encoder block attends to the corresponding iteration of the base pairing probability matrix upon input into the encoder block.

23. The method of any one of claims 1-22, further comprising padding the nucleic acid sequence for the target-guide scaffold, wherein the padding comprises adding one or more filler nucleotides to the nucleic acid sequence until the nucleic acid sequence satisfies a threshold number of nucleotide positions.

24. The method of any one of claims 1-23, further comprising padding the base pairing probability matrix, wherein the padding comprises adding one or more filler nucleotides to the base pairing probability matrix until a dimension of the base pairing probability matrix satisfies a threshold number of nucleotide positions.

25. The method of claim 23 or 24, wherein the threshold number of positions comprises at least 100 positions.

26. The method of claim 23 or 24, wherein the threshold number of positions consists of from 100 to 300 positions.

27. The method of any one of claims 23-26, wherein the nucleic acid sequence for the target-guide scaffold further comprises a concatenation junction between the first component corresponding to the gRNA and the second component corresponding to the target RNA, and wherein the padding further comprises adding the one or more filler nucleotides to a 5′ end or a 3′ end of the nucleic acid sequence for the target-guide scaffold such that the padding positions the concatenation junction at a reference position within the nucleic acid sequence for the target-guide scaffold.

28. The method of any one of claims 23-26, further comprising:
inputting, for each respective target-guide scaffold in a plurality of target-guide scaffolds, (i) respective information comprising a nucleic acid sequence for the respective target-guide scaffold, wherein the nucleic acid sequence for the respective target-guide scaffold comprises a corresponding first component for the gRNA, a corresponding second component for the target RNA, and a corresponding concatenation junction between the first component and the second component; and
padding one or more target-guide scaffolds in the plurality of target-guide scaffolds, wherein, for each respective target-guide scaffold in the plurality of target-guide scaffolds, the padding comprises adding one or more filler nucleotides to a 5′ end or a 3′ end of the nucleic acid sequence for the respective target-guide scaffold such that the padding positions the concatenation junction at a same reference position in the plurality of target-guide scaffolds.

29. The method of claim 28, wherein an alignment of the plurality of target-guide scaffolds aligns the corresponding concatenation junction of each respective target-guide scaffold in the plurality of target-guide scaffolds at the same reference position.

30. The method of any one of claims 23-29, wherein a respective filler nucleotide in the one or more filler nucleotides comprises a symbol for an unknown nucleotide N.

31. The method of any one of claims 1-31, further comprising generating, as output from the encoder block, an intermediate embedding of the nucleic acid sequence for the target-guide scaffold, wherein the intermediate embedding comprises a first component intermediate embedding for the gRNA and a second component intermediate embedding for the target RNA.

32. The method of claim 31, wherein the intermediate embedding comprises dimensions l×d, wherein l is a positive integer representing a number of nucleotide positions in the nucleic acid sequence for the target-guide scaffold.

33. The method of claim 32, wherein d is a positive integer representing a number of component encoders in the encoder block

34. The method of claim 32 or 33, wherein d is a positive integer representing a number of component decoders in the decoder block.

35. The method of any one of claims 1-34, wherein the decoder block comprises a plurality of component decoders, the second attention mechanism is a multi-head attention mechanism comprising a corresponding second plurality of attention heads, and the third attention mechanism is a multi-head attention mechanism comprising a corresponding third plurality of attention heads.

36. The method of any one of claims 1-35, wherein the third attention mechanism of the first sub-portion of the decoder block receives, as input, a first component embedding for a nucleic acid sequence of the gRNA, and the second attention mechanism of the first sub-portion of the decoder block receives, as input, a second component embedding for a nucleic acid sequence of the target RNA.

37. The method of claim 36, wherein the second attention mechanism generates, as output, a first intermediate representation of the nucleic acid sequence for the target RNA, and wherein the third attention mechanism further receives, as input, the first intermediate representation of the nucleic acid sequence for the target RNA.

38. The method of claim 37, wherein the second attention mechanism generates, as output, a second intermediate representation corresponding to the target RNA and the gRNA.

39. The method of any one of claims 1-38, wherein the second sub-portion of the decoder further comprises a position-wise feed-forward network that accepts, as input, an output from the first sub-portion, and generates, as output, the predicted set of one or more metrics for the efficiency or specificity of deamination of the target nucleotide position in the target RNA by the deamination enzyme when facilitated by hybridization of the test gRNA to the target RNA, or a representation thereof.

40. The method of any one of claims 1-39, wherein the model further comprises a fully connected layer that accepts, as input, an output from the decoder, thereby generating the predicted set of one or more metrics for the efficiency or specificity of deamination of the target nucleotide position in the target RNA by the deamination enzyme when facilitated by hybridization of the test gRNA to the target RNA.

41. The method of any one of claims 1-40, further comprising:
repeating the inputting, for each respective target-gRNA scaffold in a plurality of gRNA-target scaffolds,
thereby receiving, for each respective target-gRNA scaffold in the plurality of target-gRNA scaffolds, a corresponding predicted set of one or more metrics for the efficiency or specificity of deamination of the one or more target nucleotide positions in the target RNA by a deamination enzyme when facilitated by hybridization of the gRNA to the target RNA.

42. The method of any one of claims 1-41, wherein the model further generates an estimation of a minimum free energy (MFE) for the gRNA.

43. The method of any one of claims 1-42, wherein the deamination enzyme is an Adenosine Deaminase Acting on RNA (ADAR protein).

44. The method of any one of claims 1-43, wherein the gRNA comprises at least 25 nucleotides.

45. The method of any one of claims 1-44, wherein a respective attention mechanism is selected from the group consisting of dot product attention, query-key-value attention, Luong attention, and Bahdanau attention.

46. The method of any one of claims 1-45, wherein the model comprises at least 500,000 parameters, at least 1×10⁶ parameters, at least 1×10⁷ parameters, at least 1×10⁸ parameters, at least 1×10⁹ parameters, at least 1×10¹⁰ parameters, at least 1×10¹¹ parameters, or at least 2×10¹¹ parameters.

47. The method of any one of claims 1-46, further comprising synthesizing the gRNA, after receiving the predicted set of one or more metrics for the efficiency or specificity of deamination from the model.

48. The method of claim 47, further comprising validating the synthesized gRNA using in vitro screening.

49. The method of claim 47 or 48, further comprising placing the synthesized gRNA into a delivery vector.

50. The method of any one of claims 1-49, further comprising formulating a pharmaceutical agent comprising the gRNA, after receiving the predicted set of one or more metrics for the efficiency or specificity of deamination from the model.

51. The method of claim 50, wherein the pharmaceutical agent comprises the gRNA placed within a delivery vector.

52. The method of any one of claims 1-51, further comprising administering a pharmaceutical composition comprising the gRNA to a subject.

53. A system comprising:
a processor; and
a memory storing instructions, when executed by the processor, cause the processor to perform steps comprising the method of any one of claims 1-52.

54. A non-transitory computer-readable medium storing computer code comprising instructions, when executed by one or more processors, causing the processors to perform the method of any one of claims 1-52.
